SEQUENCE LISTING (12 sequences)

SEQ ID NO: 1 - DNA, 1650 bp, Photinus pyralis
atggaagatgcgaaaaacattaaaaaaggcccggcgccgttttatccgctggaagatggcaccgcgggcgaacagctgcataaagcgatgaaacgctatgcgctggtgccgggcaccattgcgtttaccgatgcgcatattgaagtgaacattacctatgcggaatattttgaaatgagcgtgcgcctggcggaagcgatgaaacgctatggcctgaacaccaaccatcgcattgtggtgtgcagcgaaaacagcctgcagttttttatgccggtgctgggcgcgctgtttattggcgtggcggtggcgccggcgaacgatatttataacgaacgcgaactgctgaacagcatgaacattagccagccgaccgtggtgtttgtgagcaaaaaaggcctgcagaaaattctgaacgtgcagaaaaaactgccgattattcagaaaattattattatggatagcaaaaccgattatcagggctttcagagcatgtatacctttgtgaccagccatctgccgccgggctttaacgaatatgattttgtgccggaaagctttgatcgcgataaaaccattgcgctgattatgaacagcagcggcagcaccggcagcccgaaaggcgtggcgctgccgcatcgcaccgcgtgcgtgcgctttagccatgcgcgcgatccgatttttggcaaccagattattccggataccgcgattctgagcgtggtgccgtttcatcatggctttggcatgtttaccaccctgggctatctgatttgcggctttcgcgtggtgctgatgtatcgctttgaagaagaactgtttctgcgcagcctgcaggattataaaattcagagcgcgctgctggtgccgaccctgtttagcttttttgcgaaaagcaccctgattgataaatatgatctgagcaacctgcatgaaattgcgagcggcggcgcgccgctgagcaaagaagtgggcgaagcggtggcgaaacgctttcatctgccgggcattcgccagggctatggcctgaccgaaaccaccagcgcgattctgattaccccggaaggcgatgataaaccgggcgcggtgggcaaagtggtgccgttttttgaagcgaaagtggtggatctggataccggcaaaaccctgggcgtgaaccagcgcggcgaactgtgcgtgcgcggcccgatgattatgagcggctatgtgaacgatccggaagcgaccaacgcgctgattgataaagatggctggctgcatagcggcgatattgcgtattgggatgaagatgaacatttttttattgtggatcgcctgaaaagcctgattaaatataaaggctgccaggtggcgccggcggaactggaaagcattctgctgcagcatccgaacatttttgatgcgggcgtggcgggcctgccgggcgatgatgcgggcgaactgccggcggcggtggtggtgctggaacatggcaaaaccatgaccgaaaaagaaattgtggattatgtggcgagccaggtgaccaccgcgaaaaaactgcgcggcggcgtggtgtttgtggatgaagtgccgaaaggcctgaccggcaaactggatgcgcgcaaaattcgcgaaattctgattaaagcgaaaaaaggcggcaaaagcaaactg

SEQ ID NO: 2 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIFDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 3 - DNA, 1485 bp, Pichia pastoris
atggttcacttaggggcgaagaagcctcagcatagaaaaggatatctatttaatcagcttagtccagagttacgaaaagcttataaagaagtagaggcacagttcgttgtatcaactcccaggttaaagcagatagtggatcaatttgttgctgaattgaaggaaggcttgaaatcaagcagctctaacatcccaatgctacctacctgggtgatggatttccccacaggagaggaaactggagactatctcgcaattgacctgggcgggaccaatataagggttatattggtcagactgttgggaaataggaagtttgataccattcaatcaaaatatgttttgcctaaatggatcagaacctccacatcaaatgaactttggctttttattgctcaatgtgtgaagactttcattgatgaagagtttgattatagagaaagtccggaagacccaatccccttagggttcacattttcttaccctgcattccaaagtaggattaattcgggtgtcttacaacgttggacgaaaggatttgatattccggacgttgaaggccatgacgttgtccctatgctgcaagacgcattggaatcattgggattgtccgttgtggtagtggctttaattaatgacactacaggcactttggtagcttctacgtatacggatccagagactaaaatgggactgatatttggaactggtgttaatggtgcatactatgatacgataagttcggtctcaaagatatcaaatgctcttccaccagatattcaagaggatgcaagtatggccatcaactgcgaatacggtgcttttgacaataacatctcagttctacctaggaccaaatacgatgatacaattgatttagaatcgcccagaccagggcaacagtcgtatgaaaaaatgatatcaggctattatcttggagaattgttacgattggtgcttgtagatttgcaccatcaaggacacattttcaagggacaaacaatcgggaaactaaatgaaccgttcattatggatacatcctttcctgcgagaattgaagaggatccgtttgagaatctatgtgagactggagaactttttaacagcctaggaattgaaactacggttcccgaaagagaactgattagacgaatttgcgaactcataggaacaagagcagccagactgtcagtatgtagcattgccgcaatttgcaaaaaacgaggctacaagaaagcccattgtgctgctgatggctctgtcttcacccgctatccttatttccctgacagagctgcaagagcgctccgagatatattccaatggggccattcaactccggacttagttactgtagttccagcagaagatggctcgggagttggggcagctattattgctgcactgaccaaacaacggatggcaaatggtgaatccgtgggccttgacgaatatcaccctcaagatggaaaggatagtaactaa

SEQ ID NO: 4 - protein, 494 aa, Pichia pastoris
MVHLGAKKPQHRKGYLFNQLSPELRKAYKEVEAQFVVSTPRLKQIVDQFVAELKEGLKSSSSNIPMLPTWVMDFPTGEETGDYLAIDLGGTNIRVILVRLLGNRKFDTIQSKYVLPKWIRTSTSNELWLFIAQCVKTFIDEEFDYRESPEDPIPLGFTFSYPAFQSRINSGVLQRWTKGFDIPDVEGHDVVPMLQDALESLGLSVVVVALINDTTGTLVASTYTDPETKMGLIFGTGVNGAYYDTISSVSKISNALPPDIQEDASMAINCEYGAFDNNISVLPRTKYDDTIDLESPRPGQQSYEKMISGYYLGELLRLVLVDLHHQGHIFKGQTIGKLNEPFIMDTSFPARIEEDPFENLCETGELFNSLGIETTVPERELIRRICELIGTRAARLSVCSIAAICKKRGYKKAHCAADGSVFTRYPYFPDRAARALRDIFQWGHSTPDLVTVVPAEDGSGVGAAIIAALTKQRMANGESVGLDEYHPQDGKDSN

SEQ ID NO: 5 - DNA, 1461 bp, Saccharomyces cerevisiae
atggttcatttaggtccaaaaaaaccacaagccagaaagggttccatggccgatgtgccaaaggaattgatgcaacaaattgagaattttgaaaaaattttcactgttccaactgaaactttacaagccgttaccaagcatttcatttccgaattggaaaagggtttgtccaagaagggtggtaacattccaatgattccaggttgggttatggatttcccaactggtaaggaatccggtgatttcttggccattgatttgggtggtaccaacttgagagttgtcttagtcaagttgggcggtgaccgtacctttgacaccactcaatctaagtacagattaccagatgctatgagaactactcaaaatccagacgaattgtgggaatttattgccgactctttgaaagcttttattgatgagcaattcccacaaggtatctctgagccaattccattgggtttcaccttttctttcccagcttctcaaaacaaaatcaatgaaggtatcttgcaaagatggactaaaggttttgatattccaaacattgaaaaccacgatgttgttccaatgttgcaaaagcaaatcaccaagaggaatatcccaattgaagttgttgctttgataaacgacactaccggtactttggttgcttcttactacactgacccagaaactaagatgggtgttatcttcggtactggtgtcaatggtgcttactacgatgtttgttccgatatcgaaaagctacaaggaaaactatctgatgacattccaccatctgctccaatggccatcaactgtgaatacggttccttcgataatgaacatgtcgttttgccaagaactaaatacgatatcaccattgatgaagaatctccaagaccaggccaacaaacctttgaaaaaatgtcttctggttactacttaggtgaaattttgcgtttggccttgatggacatgtacaaacaaggtttcatcttcaagaaccaagacttgtctaagttcgacaagcctttcgtcatggacacttcttacccagccagaatcgaggaagatccattcgagaacctagaagataccgatgacttgttccaaaatgagttcggtatcaacactactgttcaagaacgtaaattgatcagacgtttatctgaattgattggtgctagagctgctagattgtccgtttgtggtattgctgctatctgtcaaaagagaggttacaagaccggtcacatcgctgcagacggttccgtttacaacagatacccaggtttcaaagaaaaggctgccaatgctttgaaggacatttacggctggactcaaacctcactagacgactacccaatcaagattgttcctgctgaagatggttccggtgctggtgccgctgttattgctgctttggcccaaaaaagaattgctgaaggtaagtccgttggtatcatcggtgcttaa

SEQ ID NO: 6 - protein, 486 aa, Saccharomyces cerevisiae
MVHLGPKKPQARKGSMADVPKELMQQIENFEKIFTVPTETLQAVTKHFISELEKGLSKKGGNIPMIPGWVMDFPTGKESGDFLAIDLGGTNLRVVLVKLGGDRTFDTTQSKYRLPDAMRTTQNPDELWEFIADSLKAFIDEQFPQGISEPIPLGFTFSFPASQNKINEGILQRWTKGFDIPNIENHDVVPMLQKQITKRNIPIEVVALINDTTGTLVASYYTDPETKMGVIFGTGVNGAYYDVCSDIEKLQGKLSDDIPPSAPMAINCEYGSFDNEHVVLPRTKYDITIDEESPRPGQQTFEKMSSGYYLGEILRLALMDMYKQGFIFKNQDLSKFDKPFVMDTSYPARIEEDPFENLEDTDDLFQNEFGINTTVQERKLIRRLSELIGARAARLSVCGIAAICQKRGYKTGHIAADGSVYNRYPGFKEKAANALKDIYGWTQTSLDDYPIKIVPAEDGSGAGAAVIAALAQKRIAEGKSVGIIGA

SEQ ID NO: 7 - protein, 57 aa, Artificial Sequence
  Synthetic polypeptide
VDNKFNKEQQNAFYEILHLPNLNEEQRNAFIQSLKDDPSQSANLLAEAKKLNDAQPK

SEQ ID NO: 8 - protein, 57 aa, Artificial Sequence
  Synthetic polypeptide
VDNKFNKEEEEAEEEIEELPNLNEEQEEAFIESLEDDPSQSANLLAEAKKLNDAQPK

SEQ ID NO: 9 - protein, 57 aa, Artificial Sequence
  Synthetic polypeptide
VDNKFNKERRRARREIRHLPNLNEEQRRAFIRSLRDDPSQSANLLAEAKKLNDAQPK

SEQ ID NO: 10 - protein, 24 aa, Artificial Sequence
  Synthetic polypeptide
MASSLRQILDSQKIEWRSNAGGAS

SEQ ID NO: 11 - DNA, 72 bp, Artificial Sequence
  Synthetic polynucleotide
atggctagtagcctgcgccagatcctggacagccagaaaatcgaatggcgcagcaacgctggtggtgctagt

SEQ ID NO: 12 - protein, 15 aa, Artificial Sequence
  Synthetic polypeptide
GLNDIFEAQKIEWHE

(19) United States
(12) Patent Application Publication
(10) Pub. No.: US 2017/0260580 A1
(43) Pub. Date: Sep. 14, 2017

(54) GENETIC DETECTION PLATFORM

(71) Applicant: Pharmozyme, Inc., Sunnyvale, CA (US)

(72) Inventor: Nader NOURI ZAD, Sunnyvale, CA (US)

(21) Appl. No.: 15/508,235

(22) PCT Filed: Sep. 2, 2015
(86) PCT No.: PCT/US2015/048182

§ 371 Date: Mar. 2, 2017

Related U.S. Application Data
(60) Provisional application No. 62/044,872, filed on Sep. 2, 2014.

(51) Int. Cl. C12Q 1/68; C12N 9/12; C07K 14/315; C12Q 1/66; C12N 9/02

(52) U.S. Cl. CPC C12Q 1/6869; C12Q 1/66; C12Y 113/12007; C12Y 207/07004; C12Y 207/01001; C12N 9/0069; C07K 14/315; C12N 9/1205; C12Q 1/6806; G01N 2333/90241; G01N 2333/9125; G01N 2333/91215; C07K 2319/70; C07K 2319/21

(57) ABSTRACT

Disclosed herein are methods, compositions, apparatus, systems and kits for performing polynucleotide amplification utilizing a pure polynucleotide polymerase. In some cases, disclosed herein are methods, compositions, apparatus, systems and kits for sequencing polynucleotides.

### CROSS-REFERENCE

This application claims the benefit of U.S. Provisional Application No. 62/044,872 filed Sep. 2, 2014, which is incorporated herein by reference.

### REFERENCE TO A SEQUENCE LISTING SUBMITTED VIA EFS-WEB

The content of the ASCII text file of the sequence listing named “46681-702-601-seqlist_ST25.txt” which is 26 kb in size was created on Aug. 31, 2015, and electronically submitted via EFS-Web herewith the application is incorporated herein by reference in its entirety.

### BACKGROUND OF THE INVENTION

Genetic amplification and genetic sequencing have gained considerable interest in recent years. Various methodologies for genetic amplification have been developed to facilitate such amplification. Generally, the amplification of polynucleotides (such as DNA) requires a polynucleotide strand to be amplified (target polynucleotide), short polynucleotide fragments containing sequences complementary to the target (i.e. a primer), nucleotides and an enzyme that polymerizes (i.e. covalently links) the nucleotides in a manner complementary to the target polynucleotide. During the chemical reaction, one pyrophosphate molecule is released for every nucleoside incorporated in the newly elongated polynucleotide strand.

One amplification reaction is the polynucleotide chain reaction (PCR), which employs a heat-stable DNA polymerase. An example of such a polymerase is Taq-polymerase, which was originally isolated from the bacterium Thermus aquaticus. PCR relies on thermal cycling, consisting of cycles of repeated heating and cooling of the reaction for DNA melting and enzymatic replication of the DNA. The thermal cycling is required for (a) denaturation: separation of double stranded a polynucleotide into single stranded polynucleotides which serve as a template for the association of the nucleotide that would form the complementary strand, which typically occurs at 94-98° C.; (b) annealing: lowering the temperature to allow hydrogen bonding of the primer to the separated polynucleotide strand (i.e. ssDNA), which typically occurs at 50-65° C.; and (c) elongation: allowing optimal condition for enzymatic polymerization, thus forming the complementary strand to the template polynucleotide, which typically occurs at 70-80° C. (depending on the particular polymerase used).

Currently, genetic detection has limited sensitivity due to contaminants that are present in amplification reagents (such as polynucleotide contaminants from the host genomic material and plasmid).

### SUMMARY OF THE INVENTION

Methods, compositions, reagents, devices, systems, kits, programs, business methods, reports and computer software are provided herein for polynucleotide sample preparation for their expression, amplification, sequencing, or any combination thereof.

In some aspects, the invention discloses a method for polynucleotide sequencing comprising: (a) replicating at least one target polynucleotide by: (i) hybridizing a complementary polynucleotide that is complementary to at least a portion of the at least one target polynucleotide, to the at least one target polynucleotide; (ii) hybridizing one species of nucleoside polyphosphate to the at least one target polynucleotide, wherein the nucleoside is selected from the group consisting of adenine, thymine, guanine, cytosine, and uracil; (iii) linking the one species of nucleoside polyphosphate with the complementary polynucleotide to elongate the complementary polynucleotide; (iv) releasing at least one pyrophosphate; (b) converting the at least one pyrophosphate to ATP; (c) detecting the ATP; and (d) converting an excess of the one species of nucleoside polyphosphate to a nucleotide diphosphate or to a nucleotide monophosphate by phosphorylating at least one saccharide with the excess of the one species of nucleoside polyphosphate using a saccharide phosphorylating enzyme.

In some aspects, the invention discloses a method for polynucleotide sequencing comprising: (a) replicating at least one target polynucleotide by: (i) hybridizing a complementary polynucleotide that is complementary to at least a portion of the at least one target polynucleotide, to the at least one target polynucleotide; (ii) hybridizing one species of nucleoside polyphosphate to the at least one target polynucleotide, wherein the nucleoside is selected from the group consisting of adenine, thymine, guanine, cytosine, and uracil; (iii) linking the one species of nucleoside polyphosphate with the complementary polynucleotide to elongate the complementary polynucleotide; (iv) releasing at least one pyrophosphate; (b) converting the at least one pyrophosphate to ATP; (c) detecting the ATP; and (d) inactivating an excess of the one species of nucleoside polyphosphate without using a nucleoside degrading enzyme.

In some aspects, the invention discloses a method for polynucleotide sequencing comprising: (a) replicating at least one target polynucleotide by: (i) hybridizing a complementary polynucleotide that is complementary to at least a portion of the at least one target polynucleotide, to the at least one target polynucleotide; (ii) hybridizing one species of nucleoside polyphosphate to the at least one target polynucleotide, wherein the nucleoside is selected from the group consisting of adenine, thymine, guanine, cytosine, and uracil; (iii) linking the one species of nucleoside-polyphosphate with the complementary polynucleotide to elongate the complementary polynucleotide; (iv) releasing at least one pyrophosphate; (b) converting the at least one pyrophosphate to ATP; (c) detecting the ATP; and (d) inactivating an excess of the one species of nucleoside-polyphosphate using a thermostable enzyme.

In some aspects, the invention discloses a method for polynucleotide sequencing comprising: (a) replicating at least one target polynucleotide by: (i) hybridizing a complementary polynucleotide that is complementary to at least a portion of the at least one target polynucleotide, to the at least one target polynucleotide; (ii) hybridizing one species of nucleoside polyphosphate to the at least one target polynucleotide, wherein the nucleoside is selected from the group consisting of adenine, thymine, guanine, cytosine, and uracil; wherein the one species of nucleoside polyphosphate does not comprise nucleoside-polyphosphate thiol; (iii) linking the one species of nucleoside-polyphosphate with the complementary polynucleotide to elongate the complementary polynucleotide, and (iv) releasing at least one pyrophosphate; (b) converting the at least one pyrophosphate to ATP; and (c) detecting the ATP.

In some instances, the invention discloses a method for measuring pyrophosphate released during a polynucleotide replication process comprising: (a) performing polynucleotide replication in a reaction mixture, wherein the replication results in a release of at least one pyrophosphate; (b) converting the pyrophosphate into ATP; (c) adding at least one saccharide to the reaction mixture; and (d) detecting the ATP using a luciferase that does not recognize dATP.

In some instances, the invention also discloses a method for measuring pyrophosphate released during a polynucleotide replication process comprising: (a) performing polynucleotide replication in the presence of at least one saccharide, wherein the replication results in a release of at least one pyrophosphate; (b) converting the pyrophosphate into ATP; and (c) detecting the ATP using a luciferase that does not recognize dATP.

In some instances, the invention further discloses a method for monitoring a rate of phosphorylating saccharides comprising: (a) reacting a saccharide phosphorylating enzyme construct with a nucleoside-polyphosphate and at least one saccharide, wherein the saccharide phosphorylating enzyme construct comprises an albumin binding moiety; and (b) measuring an amount of at least one reaction product.

In some aspects, the invention discloses construct designs pertaining to one or more of the enzymes or proteins disclosed herein. In some instances, the invention discloses a polynucleotide amplification enzyme construct comprising an albumin binding moiety, wherein the polynucleotide amplification enzyme amplifies a target polynucleotide in presence of at least one nucleoside-polyphosphate.

In some instances, the invention discloses a saccharide phosphorylating enzyme construct comprising at least one moiety that has a binding affinity to at least two species of nucleoside polyphosphate; wherein said binding affinity to at least two species of nucleoside polyphosphate ranges from at least 1 micromolar to at most 250 micromolar (μM); wherein the nucleoside is selected from the group consisting of adenine, thymine, guanine, cytosine, and uracil; and wherein the saccharide phosphorylating enzyme construct phosphorylates at least one saccharide to produce at least one phosphorylated saccharide.

In some instances, the invention discloses a saccharide phosphorylating enzyme construct comprising at least one moiety that binds albumin, wherein the saccharide phosphorylating enzyme construct phosphorylates at least one saccharide to produce at least one phosphorylated saccharide; wherein the dissociation constant of the saccharide phosphorylating enzyme construct to albumin is 1.5 nanomolar or less.

In some aspects, the invention discloses saccharide phosphorylating enzyme construct comprising a saccharide phosphorylating enzyme and at least one albumin binding site. In some aspects, the invention also discloses bioluminescent enzyme construct comprising a bioluminescent enzyme and at least one albumin binding site.

In some aspects, the invention discloses bioluminescent enzyme that facilitates a bioluminescent reaction and does not recognize dATP as a substrate. In some aspects, the invention discloses saccharide phosphorylating enzyme that facilitates a saccharide phosphorylation reaction at a temperature of above 50 degrees Celsius. In some aspects, the invention discloses bioluminescent enzyme that facilitates a bioluminescent reaction at a temperature of above 50 degrees Celsius.

In some aspects, the invention discloses an albumin affinity separation method for enzyme purification comprising: (a) forming a protein construct comprising a target enzyme bound to an albumin binding moiety; (b) contacting the protein construct with albumin to form an albumin molecular complex; (c) separating the protein construct; and (d) retrieving the protein construct from the albumin molecular complex, wherein the protein construct retains activity of the target enzyme.

In some aspects, the invention discloses kits for use with one or more of the methods described herein. In some cases, the invention discloses a kit for polynucleotide sequencing comprising: (a) polynucleotide amplification reagent; (b) saccharide phosphorylating enzyme; and (c) bioluminescent enzyme; wherein the kit excludes a nucleotide degrading enzyme.

In some cases, the invention discloses a kit for polynucleotide sequencing comprising: (a) polynucleotide amplification reagent; and (b) bioluminescent enzyme that does not recognize dATP; wherein the kit excludes a bioluminescent enzyme that recognizes dATP.

In some instances, the invention discloses a kit for polynucleotide sequencing comprising: (a) a polynucleotide amplification reagent; and (b) a bioluminescent enzyme that is stable above 50 degrees Celsius, wherein the kit excludes a bioluminescent enzyme that is stable up to 50 degrees Celsius.

In some instances, the invention discloses a kit for polynucleotide sequencing comprising: (a) polynucleotide amplification reagent; (b) a thermostable enzyme which inactivates at least one nucleoside-polyphosphate; and (c) a bioluminescent enzyme.

In some instances, the invention discloses a method for measuring pyrophosphate released during a polynucleotide replication process comprising: (a) performing polynucleotide replication in a reaction mixture, wherein the replication results in a release of at least on pyrophosphate; (b) converting the pyrophosphate into ATP; (c) adding at least one saccharide to the reaction mixture; and (d) detecting the ATP using a thermostable luciferase that has an impaired recognition of dATP. The adding at least one saccharide can follow the polynucleotide replication. The saccharide can be phosphorylated. The phosphorylation of the saccharide can quench an excess of nucleotides. The phosphorylation of the saccharide can comprise using a saccharide phosphorylating enzyme. The thermostable luciferase can be a thermostable firefly luciferase. The thermostable luciferase can comprise a modification at one or more positions corresponding to amino acid residues T214, I232, F295, E354, I423, D436, L530, and L550 of SEQ ID NO: 2. The modifications can include T214A, I232A, F295L, E354K, I423L, D436G, L530R, and L550V. The modifications can include T214A, I232A, F295L, I423L, and L550V. The impaired recognition of the thermostable luciferase can be a decrease in affinity toward dATP. The thermostable luciferase may not recognize dATP and recognize ATP. The thermostable luciferase can further comprise a binding protein selected from albumin binding protein or Z domain. The converting can be conducted in the presence of ATP sulfurylase. The ATP sulfurylase can be thermostable. The polynucleotide replication can further comprise the steps of (a) hybridizing a complementary polynucleotide that is complementary to at least a portion of at least one target polynucleotide to the at least one target polynucleotide; (b) hybridizing one species of nucleoside polyphosphate to the at least one target polynucleotide, wherein the nucleoside is selected from the group consisting of adenine, thymine, guanine, cytosine, and uracil; and (c) linking the one species of nucleoside polyphosphate with the complementary polynucleotide to elongate the complementary polynucleotide. The polynucleotide replication can be performed at a temperature greater than 50 degrees Celsius. The polynucleotide replication can be performed at a temperature that is at least 50 degrees Celsius, at least 55 degrees Celsius, at least 60 degrees Celsius, at least 65 degrees Celsius, or at least 70 degrees Celsius. The polynucleotide replication can be conducted in the presence of a polymerase. The polymerase can be Taq polymerase. The Taq polymerase can be native Taq polymerase, recombinant Taq polymerase, or modified Taq polymerase. The method can exclude the use of a single strand binding protein (SSB). The saccharide phosphorylating enzyme can be hexokinase. The hexokinase can be a modified hexokinase further comprising a binding protein selected from albumin binding protein or Z domain. The hexokinase can be expressed in Saccharomyces cerevisiae, Pichia pastoris, or E. coli. The hexokinase can be a thermostable hexokinase. The albumin binding protein can comprise ABP (121aa), BB (214aa), ABD (46aa), ADB1 binding site, ADB2 binding site, or ADB3 binding site. The ABD to albumin affinity can be 1.5 nanomolar or less. The albumin can be serum albumin. The albumin can be human serum albumin. The hexokinase, the luciferase or the ATP sulfurylase can be chemically modified. The chemically modified hexokinase, luciferase, or ATP sulfurylase can comprise chemical neutralization or chemical acidification of the basic side chains of the hexokinase, luciferase or sulfurylase. The chemically modified hexokinase, luciferase, or ATP sulfurylase can comprise acetylation or citraconylation.

In some instances, the invention discloses a thermostable luciferase that facilitates a bioluminescent reaction and has an impaired recognition of dATP as a substrate. The thermostable luciferase can comprise a modification at one or more positions corresponding to amino acid residues T214, I232, F295, E354, I423, D436, L530, and L550 of SEQ ID NO: 2. The modifications can include T214A, I232A, F295L, E354K, I423L, D436G, L530R, and L550V. The modifications can include T214A, I232A, F295L, I423L, and L550V. The thermostable luciferase can be further chemically modified. The chemically modified luciferase can comprise chemical neutralization or chemical acidification of the basic side chains of luciferase. The chemically modified luciferase can comprise acetylation or citraconylation. The thermostable luciferase can be used in a method described herein.

In some instances, the invention discloses a bioluminescent enzyme construct comprising a bioluminescent enzyme and a binding moiety selected from albumin binding protein or Z domain. The bioluminescent enzyme can be luciferase. The luciferase can comprise a modification at one or more positions corresponding to amino acid residues T214, I232, F295, E354, I423, D436, L530, and L550 of SEQ ID NO: 2. The modifications can include T214A, I232A, F295L, E354K, I423L, D436G, L530R, and L550V. The modifications can include T214A, I232A, F295L, I423L, and L550V. The albumin binding protein can comprise BB, ABD, ABP, ABD1, ABD2, or ABD3. The enzyme construct can comprise a His(6) moiety. The binding moiety sequence can be connected at the 5′ of the bioluminescent enzyme sequence and further connected to the 3′ of the His(6) sequence, or can be connected at the 5′ of the HIS(6) sequence and further connected to the 3′ of the bioluminescent enzyme sequence. the HIS(6) sequence can be connected at the 5′ of the bioluminescent enzyme sequence and further connected to the 3′ of the binding moiety sequence, or can be connected at the 5′ of the binding moiety sequence and further connected to the 3′ of the bioluminescent enzyme sequence. The bioluminescent enzyme sequence can be connected at the 5′ of the binding moiety sequence and further connected to the 3′ of the His(6) sequence, or can be connected at the 5′ of the HIS(6) sequence and further connected to the 3′ of the binding moiety sequence.

In some instances, the invention discloses a saccharide phosphorylating enzyme construct comprising a binding moiety selected from albumin binding protein or Z domain, wherein the saccharide phosphorylating enzyme phosphorylates at least one saccharide to produce at least one phosphorylated saccharide. The saccharide phosphorylating enzyme can be hexokinase. The albumin binding protein can comprise ABP (121aa), BB (214aa), ABD (46aa), ADB1 binding site, ADB2 binding site, or ADB3 binding site. The ABD to albumin affinity can be 1.5 nanomolar or less. The albumin can be human serum albumin or bovine serum albumin. The enzyme construct can further comprise a HIS(6) moiety. The enzyme construct can comprise a construct depicted in FIG. 5, 19 or 20C. The enzyme construct can be expressed in Saccharomyces cerevisiae, Pichia pastoris or E. coli.

In some instances, the invention discloses a saccharide phosphorylating enzyme comprising a binding moiety selected from albumin binding protein or Z domain, wherein the enzyme has similar binding affinities for at least two nucleotides selected from the list consisting of dTTP, dCTP, dGTP, dUTP and dATP. The enzyme can phosphorylate at least one saccharide to produce at least one phosphorylated saccharide. Similar binding affinities can range from at least 1 micromolar to at most 250 micromolar. The enzyme can react the nucleotides with at least one saccharide with a similar efficiency. The enzyme can react the nucleotides with at least one saccharide in a similar rate. The saccharide can be hexose. The hexose can be selected from the group consisting of glucose, allose, altrose, mannose, gulose, idose, galactose and talose. The enzyme can be hexokinase. Hexokinase can comprise a construct described herein. Hexokinase can be further chemically modified. The chemically modified hexokinase can comprise chemical neutralization or chemical acidification of the basic side chains of luciferase. The chemically modified hexokinase can comprise acetylation or citraconylation. The chemically modified hexokinase can be a thermostable hexokinase. The saccharide phosphorylating enzyme can be used in a method described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an overview of a method, a system and an apparatus disclosed herein.

FIG. 2 shows a purification method disclosed herein. Titles on columns designate the target entity (i.e. purification tag) that the column has affinity for.

FIG. 3 depicts the constructs of the modified Taq-polymerase disclosed herein.

FIG. 4 illustrates a sequencing method disclosed herein.

FIG. 5 depicts the constructs of the modified hexokinase disclosed herein.

FIG. 6 illustrates various albumin binding sites in Streptococcal Protein G.

FIG. 7 illustrates the constructs of the modified luciferase disclosed herein.

FIG. 8 illustrates a diagram of the computer system disclosed herein.

FIG. 9A-FIG. 9C illustrate the error prone PCR method for generating the modified hexokinase.

FIG. 10A-FIG. 10B illustrate the error prone PCR method for generating the modified luciferase.

FIG. 11A-FIG. 11F show gel electrophoresis of the Taq polymerase-ABS construct following purification from either E. coli or yeast cells.

FIG. 12 illustrates an exemplary PCR mutagenesis of luciferase.

FIG. 13A-FIG. 13D depict exemplary luciferase constructs described herein.

FIG. 14 shows an illustrative SDS-PAGE on modified luciferases with different mutations.

FIG. 15 shows luciferase activity for ATP with modified luciferases described herein and Promega Luciferase at various temperatures. Promega Luciferase is used as a control.

FIG. 16A-FIG. 16C show the dATP activity of exemplary modified luciferase as a percentage of its ATP activity at various temperatures. Promega Luciferase is used as a control.

FIG. 17 illustrates comparison of luciferase activity as a percentage of its original activity at 27° C. between Promega Luciferase and an exemplary modified luciferase described herein.

FIG. 18 illustrates an exemplary PCR mutagenesis of hexokinase.

FIG. 19 illustrates exemplary hexokinase constructs described herein.

FIG. 20A-FIG. 20C illustrate exemplary luciferase and hexokinase construct designs comprising Z-basic tag.

FIG. 21A-FIG. 21B illustrate apyrase and hexokinase activity on dATP at 27° C. (A) and 50° C. (B).

FIG. 22A-FIG. 22B show apyrase and hexokinase activity on dNTP at 27° C. (A) and 50° C. (B).

### DETAILED DESCRIPTION OF THE INVENTION

Methods, compositions, reagents, devices, systems, kits, programs, business methods, reports and computer software are provided herein for polynucleotide sample preparation for their expression, amplification, sequencing, or any combination thereof. The methods, compositions and reagents find use in a number of applications, including, for example in polynucleotide sample preparation for their expression, amplification, sequencing, or any combination thereof. In addition, devices, systems, kits, programs, business methods, reports and computer software thereof may find use in practicing the subject methods, and may use the compositions and reagents provided. These and other objects, advantages, and features of the invention will become apparent to those persons skilled in the art upon reading the details of the methods, compositions, reagents, devices, systems, kits, programs, business methods, reports and computer software as more fully described below.

Before the present methods, compositions, reagents, devices, systems, kits, programs, business methods, reports or computer software are described, it is to be understood that this invention is not limited to the particular methods, compositions, reagents, devices, systems, kits, programs, business methods, reports or computer software described, as such may, of course, vary. It is also to be understood that the terminology used herein is for the purpose of describing particular embodiments only, and is not intended to be limiting, since the scope of the present invention will be limited only by the appended claims.

Where values are provided, it is understood that each value is accurate to the tenth of the unit (i.e. +/−0.1 unit) unless the context clearly dictates otherwise. Each smaller range between any stated value or intervening value in a stated range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells, reference to “the polynucleotide” includes reference to one or more polynucleotides and equivalents thereof; and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### GENERAL DESCRIPTION

Disclosed herein are methods, compositions, apparatus, systems, and kits for sequencing nucleic acid based on the detection of pyrophosphate that is released on incorporation of a nucleotide into a growing polynucleotide chain, during polynucleotide strand synthesis.

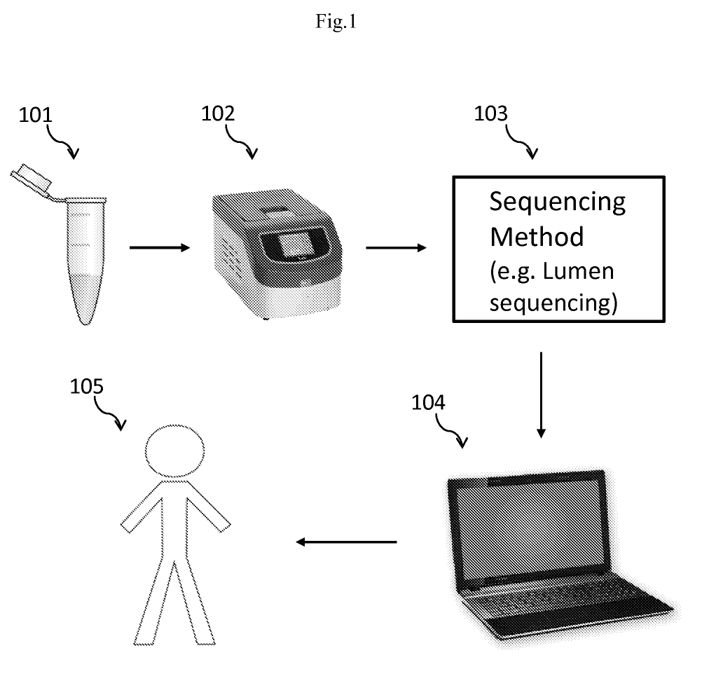

The specification facilitates amplification and/or sequencing of a target sample containing a target polynucleotide (e.g. DNA) from a subject as illustrated in FIG. 1. The sample may contain any sample that contains the target polynucleotide. The sample may be a bodily sample such as any bodily fluid, body part or tissue. For example, the sample may comprise blood, hair, skin, amniotic fluid, cells (e.g. cheek cells). The subject may be a human or an animal. The animal can be a mammal. The animal can be a pet, a wild animal, a farm animal or a laboratory animal. In some examples, the sample is inserted into a polynucleotide amplifier. The sample can subsequently undergo polynucleotide sequencing in a polynucleotide sequencer. The data from the polynucleotide sequencer can be transmitted. The data from the polynucleotide sequencer can be further analyzed. The raw or analyzed data can be delivered, transmitted, or reported to the requesting party. The requesting party may be the subject, a laboratory, a governmental entity, a hospital, a law enforcement facility, a physician, a health related facility, or any requesting party. The sample may be amplified and/or sequenced in exchange for a fee. The raw or analyzed data can be delivered, transmitted, or reported in exchange for a fee.

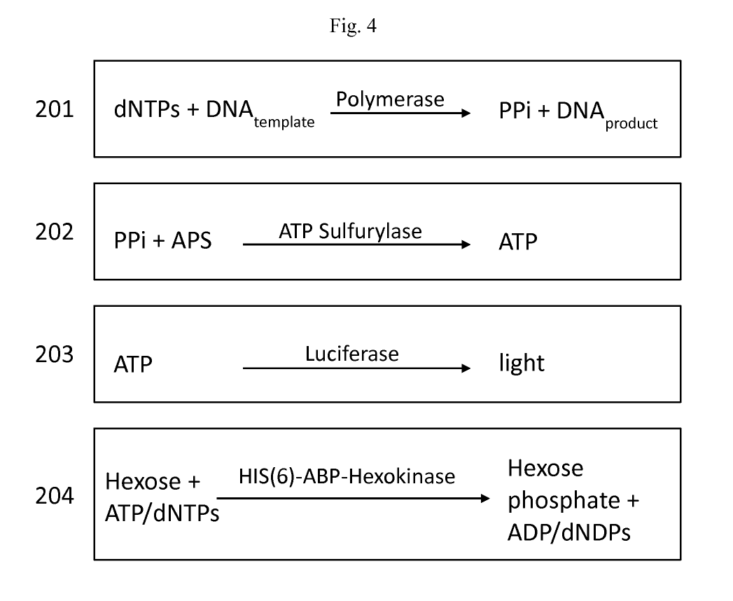

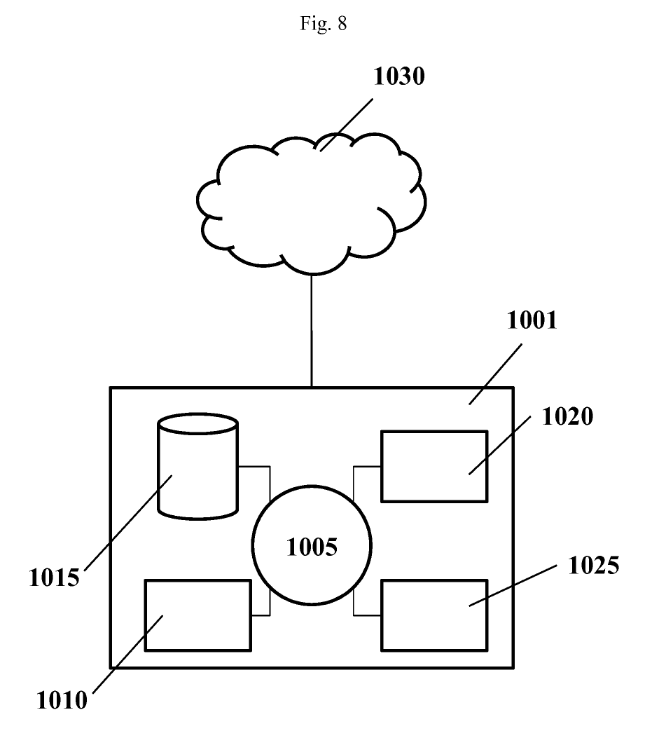

In some examples, 101 illustrates a sample input for the DNA sequencing device 102. 101 further contains reagents and components that facilitate the sequencing reaction. 102 carries out a sequencing method disclosed herein 103 (e.g. Lumen sequencing). In some cases, 102 further comprises a luminometer capable of measuring the production of light during the reaction. Sometimes, 103 is as described in FIG. 4. Upon completion of 103, the results can be transmitted to a computer 104. In some cases, 104 is as described in FIG. 8. 105 can be operatively coupled to a computer network (e.g. Internet, an internet, extranet, telecommunication network, or a data network). 104 can contain an electronic storage system. In some cases, the electronic storage system include non-transitory storage modules such as any or all of the tangible memory of the computers, processors or the like, or associated modules thereof, such as various semiconductor memories, tape drives, disk drives and the like; or an external storage devices, such as for example, hard disks, external hard drives, CDs, DVDs, flash drives, or the like. 104 can analyze the data, and can transmit the data to a user 105. The transmission of the data can be via the computer network (e.g. Internet, an internet, extranet, telecommunication network, or a data network). The transmission of the data can be via the electronic storage system (e.g. non-transitory storage modules or externally storage devices). The data can be transmitted visually, such as for example, shown on a screen that is part of 104 or as an externally connected screen, or by sound. The user 105 can be an operator or an end user. In some cases, the end user is a lab technician, a physician, a patient, a researcher, or a customer.

The sequencing method benefits from the polynucleotide (e.g. DNA) replication reaction. The method may utilize a target polynucleotide to be replicated and amplified, a polynucleotide primer that associates with the target polynucleotide, nucleotides to be incorporated into the growing chain in a manner complementary to the target polynucleotide (introduced one by one in a controlled manner), and an enzyme that facilitates the covalent bonding of polyphosphate nucleosides (e.g. nucleotides) to the newly formed polynucleotide that is complementary to the target polynucleotide. During the formation of a covalent bond between a polyphosphate nucleoside and the growing strand that is complementary to the target polynucleotide, pyrophosphate is released. By controlling the specific type of nucleotide that is introduced to the polynucleotide polymerization reaction, and monitoring the release of pyrophosphate, the sequence of the target polynucleotide may be determined. Pyrophosphate (PPi) is released only when the introduced polyphosphate nucleoside (e.g. nucleotide) is complementary to the position of chain elongation, and the covalent bonding of the nucleoside occurs. The amount of PPi released is equivalent to the number of bases incorporated. For example, when one introduces a polyphosphate nucleoside (e.g. nucleotide) that is complementary to one nucleotide on the target polynucleotide at the position where a nucleotide is to be incorporated into the elongated polynucleotide chain, and that nucleotide is incorporated into the growing chain via a covalent bond (with the aid of a polymerase enzyme), one pyrophosphate molecule is released. When one introduces a polyphosphate nucleoside (e.g. nucleotide) that is complementary to two or more sequential nucleotide bases at the position of chain growth on the target polynucleotide, two or more pyrophosphate molecules are respectively released. When one introduces a polyphosphate nucleoside (e.g. nucleotide) that is not complementary to a nucleotide base to be incorporated at the position of chain elongation on the target polynucleotide, no pyrophosphate molecule is released. The target protein may be a polynucleotide replicating enzyme, a polynucleotide amplification enzyme, a saccharide phosphorylating enzyme, a luminescent enzyme, a bioluminescent enzyme or a light emitting protein. In some examples, the target protein may be luciferase, DNA polymerase or hexokinase.

In some cases, the species of nucleoside-polyphosphate molecule are incorporated one by one. The introduction of the species of nucleoside-polyphosphate molecule can be controlled. Further, the introduction of the type of the species of nucleotide-polyphosphate molecule can be controlled one by one. Nucleotide polyphosphate can comprise one, two, three, four, five or more phosphates.

The primer can be introduced to initiate the replication reaction. The target polynucleotide may be double stranded, and the method can incorporate separation of the double stranded polynucleotide. In some examples, the target polynucleotide is single stranded.

Further, the nucleotide can comprise dATP, dTTP, dCTP, dGTP or dUTP. Sometimes, the nucleotide does not comprise a dATP analogue, but rather dATP. Occasionally, the method does not utilize single strand binding protein (SSB). Sometimes, the method excludes at least one of a nucleotide degrading enzyme, a labeled nucleoside, a labeled polynucleotide or a polynucleotide fragmentation step.

The polynucleotide sequencing may be performed at a rate of at least 0.5, 1, 2, 3, 4, 5 times or greater as compared to conventional sequencing methodologies known in the art. The sequencing may be performed at a rate of at most 0.5 1, 2, 3, 4, 5 times or smaller as compared to conventional sequencing methodologies known in the art.

The method may be conducted at an elevated temperature. The elevated temperature can be greater than 50 degrees Celsius. The elevated temperature may be from at least 50 degrees Celsius to at most 55 degrees Celsius.

The method may further include conversion of pyrophosphate to ATP. Such conversion may be facilitated by ATP sulfurylase which converts pyrophosphate to ATP in the presence of adenosine 5′ phosphosulfate. Such ATP may be detected. For example, ATP may be optically detected utilizing luciferase in the presence of luciferin and oxygen. In some instances, the luciferase is a mutated luciferase that does not recognize dATP.

Excess of nucleotides may be degraded following introduction of a polyphosphate nucleoside (e.g. nucleotide) and lapse of time for potential hybridization and incorporation of the base into the replicating polynucleotide strand. In some examples, the excess polyphosphate nucleoside (e.g. nucleotide) is degraded by a nucleotide degrading enzyme (e.g. apyrase). The excess polyphosphate nucleoside (e.g. nucleotide) can also be converted into a phosphorylated saccharide using a phosphorylating enzyme such as a saccharide phosphorylating enzyme (e.g. hexokinase). In some instances, the saccharide phosphorylating enzyme is a modified saccharide phosphorylating enzyme that utilizes at least two, three or at least four of the nucleotides (e.g. ATP, TTP, CTP, GTP, UTP, dATP, dTTP, dCTP, dGTP, or dUTP) in a similar manner in the saccharide phosphorylation reaction. In some examples, the affinity of the saccharide phosphorylating enzyme to the various nucleotides (e.g. ATP, TTP, CTP, GTP, UTP, dATP, dTTP, dCTP, dGTP, or dUTP) is similar. A similar affinity may vary by at most 0.1, 0.5, 1, 2, 3, 4, 5, 6, 7, 8, 9, 10, 15, 20 or 25 percent (%) or less among the various nucleotides binding to the saccharide phosphorylating enzyme. In some examples, the binding constants of the saccharide phosphorylating enzyme to the various nucleotides (e.g. ATP, TTP, CTP, GTP, UTP, dATP, dTTP, dCTP, dGTP, or dUTP) are similar. A similar binding constant may vary by at most 0.1, 0.5, 1, 2, 3, 4, 5, 6, 7, 8, 9, 10, 15, 20 or 25 percent (%) or less among the various nucleotides binding to the saccharide phosphorylating enzyme. In some embodiments, the method does not utilize 2′-deoxyadenosine-5′-O-(1-Thiotriphosphate) (also known as alpha thiol dATP, dATPaS).

The polynucleotide synthesis may be facilitated by a modified polymerase. Sometimes, the modified polymerase is modified Taq polymerase. The modification may be chemical modification. The modification may be an enzymatic construct that incorporates a protein able to bind albumin and Taq polymerase. Such albumin may be bovine serum albumin or human serum albumin. The sequencing method may be any sequencing method employed in the art. Additionally, the sequencing method may incorporate unique enzyme constructs or modified enzymes as explained herein.

In some aspects, the polynucleotide synthesis is facilitated by any polymerase enzyme which facilitates a polynucleotide elongation reaction coupled with the release of pyrophosphate. The sequencing method may incorporate any enzyme that facilitates conversion of pyrophosphate to ATP (such as ATP sulfurylase), and any enzyme that facilitates the detection of ATP (such as luciferase), as well as a phosphorylating enzyme that phosphorylates a saccharide by utilizing the dNTPs in a similar manner, thus degrading the surplus of dNTPs fed into the reaction during the polynucleotide polymerization stage. Sometimes, the phosphorylating enzyme is hexokinase mutant that recognizes at least two, three, four or five of the nucleotides in a similar manner.

In some aspects, the polynucleotide synthesis is facilitated by any polymerase enzyme that facilitates a polynucleotide elongation reaction, which releases pyrophosphate. The sequencing method may incorporate any enzyme that facilitates conversion of pyrophosphate to ATP (such as ATP sulfurylase), any enzyme that either utilizes an excess of dNTPs to convert saccharide into phosphorylated saccharide or degrades an excess of dNTPs fed into the reaction during the polynucleotide polymerization stage, and a luciferase mutant that is able to recognize ATP, but has reduced recognition to dATP or does not recognize dATP.

Sometimes one or more of the abovementioned enzymes is chemically modified. For example, one or more of the abovementioned enzymes is thermophilic. In one or more of the above-mentioned enzymes at least one positively charged amino acid can be neutralized or acidified. In one or more of the abovementioned enzymes, most or all of the positively charged amino acid can be neutralized or acidified. At least one of luciferase, hexokinase and ATP sulfurylase, can be chemically modified. Apyrase can also be chemically modified.

One or more of the abovementioned enzymes can be modified to incorporate a HIS (e.g., HIS(6)) construct, an albumin binding domain (ABS), biotin-tag, Z domain (or Z domain moiety), or any combinations thereof. One or more of the abovementioned enzymes can be modified to incorporate an albumin binding domain (i.e. ABS). One or more of the abovementioned enzymes can be modified to incorporate both HIS(6) moiety and an albumin binding domain. One or more of the abovementioned enzymes can be modified to incorporate a Z domain (see infra). One or more of the abovementioned enzymes can be modified to incorporate both Z domain and an albumin binding domain. One or more of the abovementioned enzymes can be modified to incorporate ABP-Z. At least one of luciferase, hexokinase and ATP sulfurylase, is thus modified. Apyrase may also be thus modified.

ABP-Z is a modified ABP that further contains a Z domain from SpA as a second binding site.

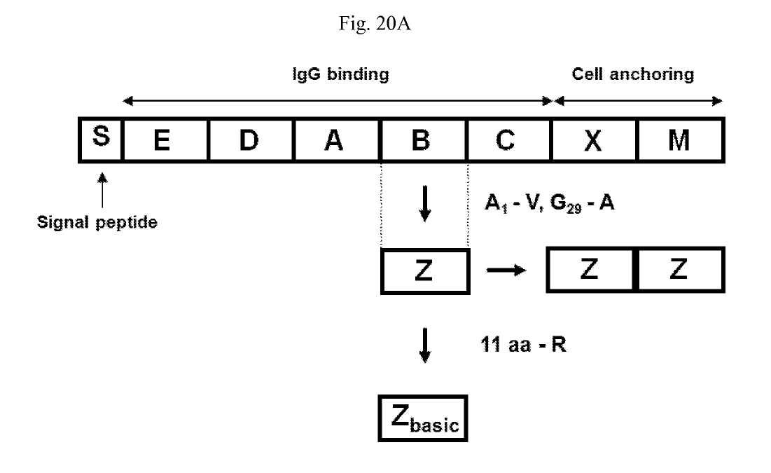

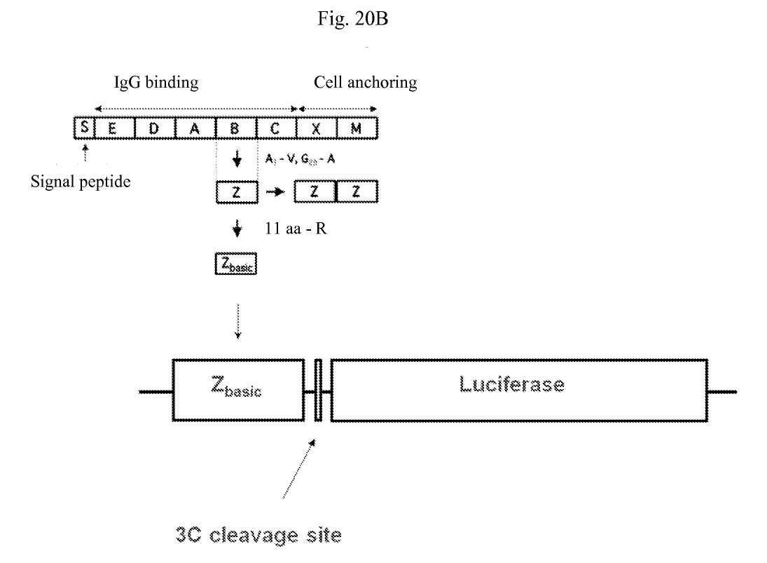

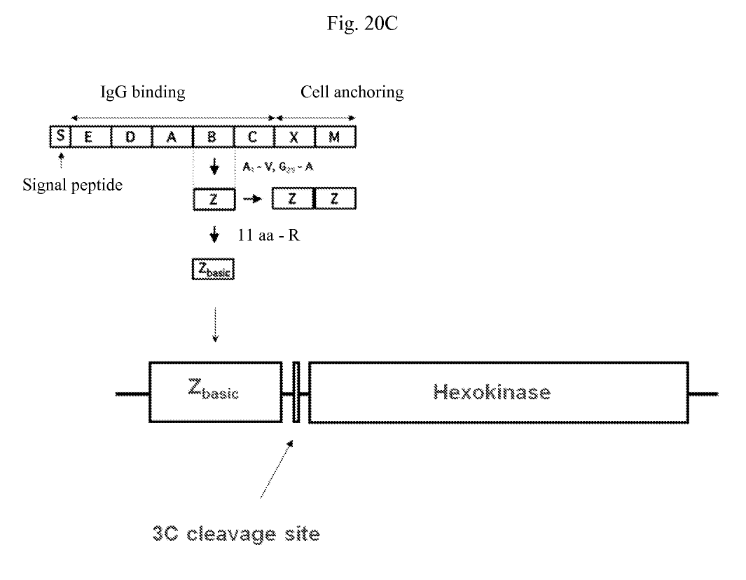

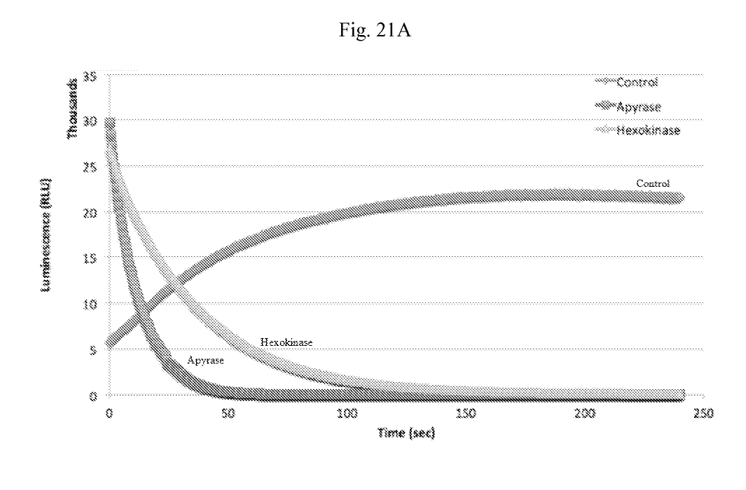

Sometimes, the Z domain (or Z domain moiety) is referred to as Z domain wild type, Zacid₂, or Zbasic₂ (see FIG. 20A). The Z domain (or Z domain moiety) can have the sequences VDNKFNKEQQNAFYEILHLPNLNEEQRNAFIQSLKDDPSQSANLLAEAKKLNDAQPK (SEQ ID NO: 7) (Z domain wild-type), VDNKFNKEEEEAEEEIEELPNLNEEQEEAFIESLEDDPSQSANLLAEAKKLNDAQPK (SEQ ID NO: 8) (sometimes can be referred to as Zacid₂), or VDNKFNKERRRARREIRHLPNLNEEQRRAFIRSLRDDPSQSANLLAEAKKLNDAQPK (SEQ ID NO: 9) (sometimes can be referred to as Zbasic₂). The Z domain (or Z domain moiety) can have the sequence VDNKFNKEQQNAFYEILHLPNLNEEQRNAFIQSLKDDPSQSANLLAEAKKLNDAQPK (SEQ ID NO: 7) (Z domain wild-type). The Z domain (or Z domain moiety) can have the sequence VDNKFNKEEEEAEEEIEELPNLNEEQEEAFIESLEDDPSQSANLLAEAKKLNDAQPK (SEQ ID NO: 8) (sometimes can be referred to as Zacid₂). The Z domain (or Z domain moiety) can have the sequence VDNKFNKERRRARREIRHLPNLNEEQRRAFIRSLRDDPSQSANLLAEAKKLNDAQPK (SEQ ID NO: 9) (sometimes can be referred to as Zbasic₂).

In some instances, the sequencing method utilizes an enzyme that phosphorylates a saccharide, forming saccharide phosphate. The saccharide can be a monosaccharide, disaccharide, oligosaccharide or polysaccharide. In some instances, the saccharide is a monosaccharide. The mono saccharide can be hexose or pentose. In some examples, the monosaccharide is a hexose. Sometimes, the hexose is glucose. The enzyme can be a hexokinase. In some instances, the sequencing method further utilizes a polymerase, such as a Taq polymerase, and an enzyme that utilizes pyrophosphate during ATP conversion. The ATP converting enzyme can be an ATP sulfurylase, or a pyruvate orthophosphate dikinase. In some instances, the sequencing method further utilizes an enzyme to convert ATP into light. The enzyme used can be a luciferase.

The sequencing method described above can utilize a modified hexokinase. In some cases, the sequencing method containing the modified hexokinase is referred herein as “Lumen sequencing.” The results of the sequencing method can then be analyzed and submitted to an end user.

### General Procedure for Production of the Target Protein Construct.

In some aspects, any of the above-mentioned protein constructs are produced using vector transformation. Sometimes, the above-mentioned protein constructs are produced by covalently linking individual polynucleotide (e.g. DNA) sequences encoding for protein segments comprising the protein construct. Any of the above-mentioned protein constructs can be produced using a protein synthesizer. Any of the above-mentioned protein constructs can be fabricated using any combination of the methods mentioned in this paragraph.

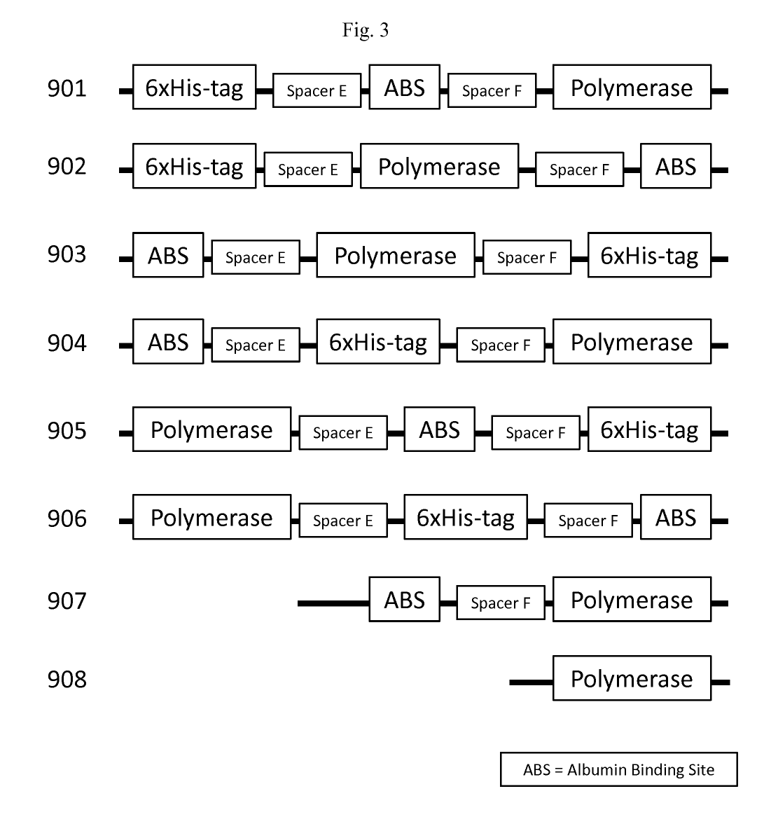

In some instances, any of the above-mentioned protein constructs are produced by a bioengineered host. Such bioengineering may be effectuated by transforming the host with a suitable vector that carries the desired polynucleotide sequence of the target protein. The vector transformation can cause the target polynucleotide to be expressed into target protein by using the protein expression mechanism of the host. The vector construct can be engineered such that when the sequence carried by the vector is expressed into a protein, the protein would correspond to the target protein (e.g., ABS construct which are covalently linked). The covalent linkage may be direct or indirect (e.g. though a spacer). Sometimes the target protein is covalently linked to HIS (e.g., His(6)). The covalent linkage may be direct or indirect (e.g. though a spacer). Sometimes the target protein is covalently linked to both ABS and HIS (e.g., His(6)). The covalent linkage may be direct or indirect (e.g. though a spacer) in any order. See for example FIGS. 3, 5, 7, 13, 19, and 20 where the target proteins can be linked directly or indirectly to for example ABS such as ABP, ABP-Z or Z domain (or Z domain moiety). The ABP-Z protein may be an albumin binding protein that further contains a second binding site. The second binding site may recognize a Z domain from SpA. The target protein may be a polynucleotide replicating enzyme, a polynucleotide amplification enzyme, a saccharide phosphorylating enzyme, a luminescent enzyme, a bioluminescent enzyme or a light emitting protein. The target protein may be luciferase, Taq polymerase or hexokinase.

Exemplary vectors include, but are not limited to, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, and pYES3/CT yeast vector. In some instances, the vector is vectors pET21 from E. coli. Sometimes, the vector is pPIC9 from Pichia pastoris. In some examples, any of the above-mentioned protein construct is synthetically produced.

### General Procedure for Purification of the Target Protein Construct.

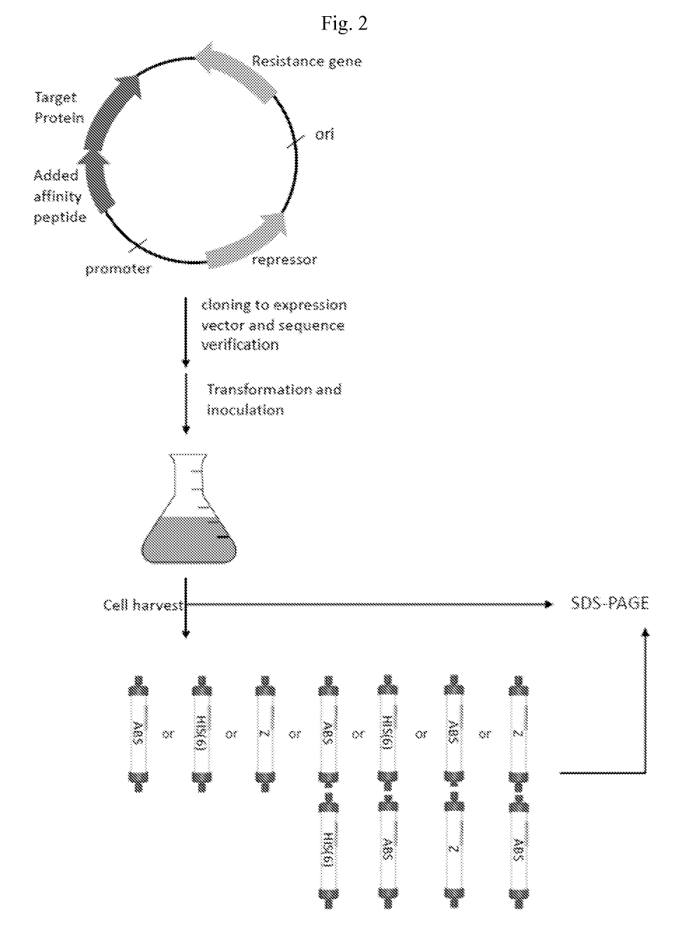

In some instances, described herein include a target protein purification method. In some cases, the purification method is as illustrated in FIG. 2. The target protein purification method comprises a native, unmodified or modified target protein. The target protein can be a polynucleotide replicating enzyme, a polynucleotide amplification enzyme, a saccharide phosphorylating enzyme, a luminescent enzyme, a bioluminescent enzyme or a light emitting protein. The target protein can be luciferase, Taq polymerase or hexokinase. The purification method further utilizes a HIS (e.g., His(6)) tag, an ABS tag, or both. Purification can be carried out using either the HIS (e.g., His(6)) tag or the ABS tag, or can be carried out in tandem with one or more purifications of the target protein using the HIS (e.g., His(6)) tag, followed by one or more purifications using the ABS tag. In some instance, purification is carried out in tandem with one or more purifications of the target protein using the ABS tag, followed by one or more purifications using the HIS (e.g., His(6)) tag. In some cases, the purification of the ABS tag involves using an albumin-affinity column or a HIS (e.g., His(6)) column. The purification of the ABS tag may involve using an albumin-affinity column and a HIS (e.g., His(6)) column. The albumin-affinity column can either precede or supersede the HIS (e.g., His(6)) column as exemplified in FIG. 2. The ABS tag may contain ABP and ABP-Z. Sometimes, the ABS tag contains ABP. In some cases, the purification of the ABS tag involves using an albumin-affinity column, or an albumin-affinity column and a Z-affinity column that utilizes ABP-Z (e.g. sites on ABS that recognizes albumin and Z-domain). The albumin-affinity column can either precede or supersede the Z-affinity column as exemplified in FIG. 2. As used herein, FIG. 2 refers to the albumin-affinity column, the HIS(6)-affinity column, and the Z-affinity column as albumin column, HIS(6) column, and Z column.

In some cases, purification with the HIS (e.g., His(6)) tag is carried out in batch mode (e.g. the use of Nickel or Cobalt-charged resin in a solution of target protein lysate) or via a column (either by gravitation filtration or by a chromatography system). Exemplary Nickel and Cobalt beads include, but are not limited to, Ni-NTA agarose (Qiagen), Ni-NTA magnetic agarose beads (Qiagen), His60 Ni Superfiow Resin (Clontech Laboratories), complete His-Tag purification resin (Roche), Dynabeads® His-Tag Isolation and Pulldown cobalt beads (Life Technologies), Dynabeads® TALON™ cobalt beads (Life Technologies), or HisPur Cobalt resin (Thermo Scientific). Exemplary Nickel and Cobalt containing columns include, but are not limited to, in-house packed Nickel or Cobalt columns, HiTrap™ Ni-NTA columns (Qiagen), Ni-NTA Superfiow columns (Qiagen), Ni-NTA Spin columns (Qiagen), His60 Ni Superfiow columns (Clontech Laboratories), or HisPur Cobalt Spin Column (Thermo Scientific).

In general, the target protein lysate is bound to either the Nickel-charged or Cobalt charged beads in a binding buffer containing a low concentration of imidazole. Imidazole competes with the His(6) tag in binding to the Nickel or Cobalt-charged beads. In some cases, the concentration of the imidazole used in the binding buffer is at most 0.01, 5, 10, 15, 20, 25, 30 millimolar (mM), or less. The concentration of the imidazole used in the binding buffer can be at least 0.01, 5, 10, 15, 20, 25, 30 millimolar (mM), or more. After the initial binding step, the beads containing the target protein is subsequently washed to remove any unbound target proteins. Upon completion of the washing step, the target protein is then eluded using an elution buffer containing a higher concentration of imidazole than used in the binding buffer. The concentration of imidazole used in the elution buffer can be at least 200, 250, 300, 350, 400, 450, 500, 550, 600, 700, 800, 900, 1000 millimolar (mM), or more.

The eluted target protein is further subjected, in some cases, to a desalting step to remove the concentration of imidazole present in the buffer. The eluted target protein can be dialyzed to remove the imidazole prior to loading onto a medium containing albumin. The albumin can be mammalian albumin (e.g. bovine or human). The albumin may be serum albumin (e.g. bovine serum albumin or human serum albumin). The medium containing albumin may be particles containing bound or unbound albumin. The particles may be magnetic particles. In some instances, the particles may contain a tag. The tag can be an optical tag (e.g. a fluorescence or phosphorescence tag). The medium containing albumin may be a solid support containing bound or unbound albumin. In some instances unbound albumin is diffused into the medium. The bound albumin may be a covalently bound albumin. The medium containing albumin may be a chromatography column, which is comprised of particles having bound albumin. The albumin can be mammalian albumin (e.g. bovine or human albumin). The albumin may be serum albumin (e.g. bovine serum albumin or human serum albumin). The medium containing albumin can be a solution comprising albumin. The medium containing albumin can be a filter comprising albumin. In some instances, the filter may be a dialysis filter.

In some instances, the binding affinity (related to one over the dissociation constant (1/K_{d})) is of at least 0.1, 0.5, 1, 1.5, 2, 2.5, 3 or more nanomolar (nM) of albumin to an albumin binding domain. In some instances, the binding affinity is of at most 0.1, 0.5, 1, 1.5, 2, 2.5, 3 or less nanomolar (nM) of albumin to an albumin binding domain. Occasionally, the binding affinity of albumin binding domain to albumin is of about 1 nanomolar (nM).

In some instances, the ABS peptide portion that is bound to the albumin is released by a change of the environment that is at least immediately adjacent to the ABS-albumin pair. The environmental change can be altering the temperature, hydrophobicity, ionic strength, conductivity or pH of the environment. A change of ionic strength can take place by alteration or addition of a salt. A change of pH may be effectuated by an addition of a base. A change of pH may be effectuated by an addition of an acid. A change of hydrophobicity may be effectuated by addition of a hydrophobic substance. The hydrophobic substance may be an alcohol such as ethanol, propanol, butanol, pentanol, hexanol, heptanol, octanol, nonanol or any combination thereof. The alcohol may comprise a linear or branched aliphatic moiety. The alcohol may comprise at least one aromatic moiety. Sometimes, the ABS peptide portion that is bound to the albumin can also be released by a competitor ABS protein, such that it competes with the ABS peptide portion for interaction with albumin.

Separation of the ABS peptide portion from the albumin may take place by washing, immersing, or eluting the complex of ABS and albumin with a solution. The solution may be a buffer solution.

The ABS purification step may be performed on a liquid chromatography system (e.g. HPLC or FPLC) using a column immobilized with albumin. The column may be immobilized with bovine or human serum albumin. The column may be immobilized with human serum albumin. The target protein may be loaded onto a column immobilized with albumin with a loading buffer containing a neutral pH. The loading buffer may contain a basic pH (e.g. about pH 7.5, pH 8.0, or pH 8.5). The column may subsequently be washed with a washing buffer containing an acidic pH. The target protein may be eluted using an elution buffer containing an acid, such as an acetic acid, at a pH of at most 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or less. Sometimes, the pH of the elution buffer is at least 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or more. In some cases, the concentration of acid used in the elution buffer is at least 200, 250, 300, 350, 400, 450, 500, 550, 600, 700, 800, 900, 1000 millimolar (mM), or more. The eluted target protein may be dialyzed into a buffer containing a neutral pH. The eluted target protein may be dialyzed into a buffer containing a basic pH (e.g. about pH 7.5, pH 8.0, or pH 8.5).

In some instances, the ABS protein further comprises a Z domain recognition site (e.g. ABP-Z). The ABP-Z purification step can be performed on a liquid chromatography system (e.g. HPLC or FPLC) using a column immobilized with protein A-derived ligand. Rhe protein A-derived ligand can be alkali-tolerant. The target protein can be loaded onto a column immobilized with an alkali-tolerant protein A-derived ligand with a loading buffer containing a neutral pH. The loading buffer can contain a basic pH (e.g. about pH 7.5, pH 8.0, or pH 8.5). The column can be subsequently washed with the loading buffer. The target protein can be eluted using an elution buffer containing an acid, such as an acetic acid, at a pH of at most 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or less. The pH of the elution buffer can be at least 1.0, 1.5, 2.0, 2.1, 2.2, 2.3, 2.4, 2.5, 2.6, 2.7, 2.8, 2.9, 3.0, 3.5, 4.0, 4.5, 5.0, 5.5, or more. The concentration of acid used in the elution buffer can be at least 200, 250, 300, 350, 400, 450, 500, 550, 600, 700, 800, 900, 1000 millimolar (mM) or more. The eluted target protein can be dialyzed into a buffer containing a neutral pH. The eluted target protein can be dialyzed into a buffer containing a basic pH (e.g. about pH 7.5, pH 8.0, or pH 8.5).

Purification with the biotin-tag (e.g., biotin binding domain tag) can involve coupling of a biotin molecule to the biotin tag in vivo or in vitro by enzymatic biotinylation prior to purification of the polymerase protein through avidin or streptavidin based method. Enzymatic biotinylation may utilize biotin ligase (BirA) to conjugate a biotin molecule to the biotin-tag. The biotin-tag (e.g., biotin binding domain tag) may be a polypeptide tag comprising the amino acid sequence selected from MASSLRQILDSQKIEWRSNAGGAS (SEQ ID NO: 10) or GLNDIFEAQKIEWHE (SEQ ID NO: 12). The biotin-tag MASSLRQILDSQKIEWRSNAGGAS (SEQ ID NO: 10) has the DNA sequence of ATGGCTAGTAGCCTGCGCCAGATCCTGGACAGCCAGAAAATCGAATGGCGCAGCAA CGCTGGTGGTGCTAGT (SEQ ID NO: 11). The biotin-tag (e.g., biotin binding domain tag) may be a polypeptide tag comprising the amino acid sequence MASSLRQILDSQKIEWRSNAGGAS (SEQ ID NO: 10). The biotin-tag (e.g., biotin binding domain tag) may be a polypeptide tag comprising the amino acid sequence GLNDIFEAQKIEWHE (SEQ ID NO: 12). The biotin-tag may be a polypeptide tag consisting of the amino acid sequence MASSLRQILDSQKIEWRSNAGGAS (SEQ ID NO: 10). The biotin-tag may be a polypeptide tag consisting of the amino acid sequence GLNDIFEAQKIEWHE (SEQ ID NO: 12). Biotinylation can be achieved in vivo or in vitro. Sometimes, biotinylation can be achieved in vivo. Sometimes, in vivo biotinylation of a Taq polymerase described herein comprises a biotin-tag of MASSLRQILDSQKIEWRSNAGGAS (SEQ ID NO: 10).

Sometimes, biotinylation can be achieved in vitro. During the biotinylation process, biotin first forms biotinoyl-5′-AMP in the presence of ATP, and biotinoyl-5′-AMP interacts with the epsilon-amine of a lysine residue within the biotin-tag to form an amide bond and this process can be facilitated by the BirA enzyme. Purification of the polymerase protein with avidin or streptavidin resin can be achieved through batch mode or may be performed on a liquid chromatography system (e.g., HPLC or FPLC) using a column immobilized with avidin or streptavidin ligands. Elution of the polymerase protein can be achieved by a change in the environment that is at least immediately adjacent to the avidin/streptavidin-biotin pair. As described supra, the environmental change can be altering the temperature, hydrophobicity, ionic strength, conductivity or pH of the environment. In some cases, elution from avidin/streptavidin resin is achieved with biotin analogs such as desthiobiotin, which competes binding of avidin or streptavidin with biotin. Sometimes, elution from avidin/streptavidin resin is achieved with an elution buffer containing a denaturing agent such as urea or guanidinium chloride, a high ionic strength buffer such as 1M NaCl, or 1M (NH₄)₂SO₄, or an elution buffer that has a pH range from about 2-about 3.

Purification with the Z domain protein (or Z domain moiety) (e.g., Z domain wild type, Zacid₂, or Zbasic₂) can comprise contacting a Taq polymerase solution comprising the Z domain with an IgG immobilized resin in either batch mode or through a column chromatography method. Sometimes, a modified Taq polymerase comprising the Z domain (e.g., Z domain-HIS-Taq) described herein can be purified using a cation exchange chromatography method. The Z domain is an engineered analogue of the IgG-binding domain B of Staphylococcal protein A (SpA) (FIG. 20A).

Specific examples of target protein constructs will be elaborated infra.

### Hexokinase

In some aspects, the invention includes a modified form of a hexokinase. Hexokinase is an enzyme that phosphorylates a six-carbon sugar (i.e. hexose), and generates hexose phosphate. Wild-type hexokinase utilizes an ATP or dATP to convert a hexose into a hexose phosphate. However, its affinity toward other dNTPs (e.g. dTTP, dCTP, dGTP or dUTP) is low. The modified hexokinase disclosed herein may differ from the wild-type in the affinity of the modified hexokinase to the various dNTPs. The modified hexokinase may have similar affinity to at least two, three, four or five of the naturally occurring nucleosides (i.e. dATP, dTTP, dCTP, dGTP and dUTP). The modified hexokinase can utilize one or more dNTPs during hexose phosphorylation. The modified hexokinase can utilize one or more of NTP or dNTPs selected from ATP, dATP, dTTP, dCTP, dGTP, and dUTP during hexose phosphorylation. In some cases, the modified hexokinase utilizes ATP, dATP, dTTP, dCTP, and dGTP during hexose phosphorylation. The usage of ATP and dNTPs by the modified hexokinase can be utilized for removal of excess ATP and dNTPs during the sequencing reactions disclosed herein. The modified hexokinase can replace a nucleotide degrading enzyme (e.g. apyrase) during the sequencing reaction disclosed herein for removal of excess ATP and dNTPs. Such novel sequencing method is called “Lumen Sequencing.”

The rate of utilizing the various dNTPs by the modified hexokinase can be evaluated using an enzyme efficiency value. As used herein, the enzyme efficiency value refers to the specificity constant of an enzyme and can be described by the ratio of the catalytic constant Kcat of the enzyme, to the Michaelis constant Km. In some cases, efficiency is used to compare the activity of an enzyme in its action against different substrates. In some cases, the efficiency value of utilizing the various dNTPs (excluding dATP) by the modified hexokinase is compared to the efficiency value of utilizing ATP/dATP. In some cases, the comparison is expressed as a ratio. In some cases, the ratio of the efficiency value of at least one of the various dNTPs (excluding dATP) to the efficiency value of ATP/dATP is at least 0.5, 0.6, 0.7, 0.8, 0.9, 1, 2, 3, 4, 5, or more. Sometimes, the ratio of the efficiency value of at least one of the various dNTPs (excluding dATP) to the efficiency value of ATP/dATP is at most 0.6, 0.7, 0.8, 0.9, 1, 2, 3, 4, 5, or less. Sometimes, the ratio of the efficiency value of at least one of the various dNTPs (excluding dATP) to the efficiency value of ATP/dATP is about 1. The various dNTPs (excluding dATP) may include dTTP, dCTP, dGTP. At times the various dNTPs may also include dUTP. At times, the various dNTPs may include non-natural or rare dNTPs.

The substrate for the modified hexokinase may be glucose, generating glucose-6-phosphate as the product. At times, substrates for the modified hexokinase include allose, altrose, mannose, gulose, idose, galactose, talose, psicose, fructose, sorbose, or tagatose. In some instances, the modified hexokinase phosphorylates one or more of the hexose substrates selected from allose, altrose, glucose, mannose, gulose, idose, galactose, talose, psicose, fructose, sorbose, and tagatose. In some instances, the modified hexokinase phosphorylates only one hexose substrate selected from allose, altrose, glucose, mannose, gulose, idose, galactose, talose, psicose, fructose, sorbose, and tagatose.

Hexoses contain two configurations, D-hexoses and L-hexoses. In some cases, the modified hexokinase disclosed herein phosphorylates D-hexose substrates. In some cases, the D-hexose substrates include D-allose, D-altrose, D-glucose, D-mannose, D-gulose, D-idose, D-galactose, D-talose, D-psicose, D-fructose, D-sorbose, or D-tagatose. In some cases, the modified hexokinase phosphorylates one or more of the D-hexose substrates selected from D-allose, D-altrose, D-glucose, D-mannose, D-gulose, D-idose, D-galactose, D-talose, D-psicose, D-fructose, D-sorbose, or D-tagatose. In some cases, the modified hexokinase phosphorylates only one D-hexose substrate selected from D-allose, D-altrose, D-glucose, D-mannose, D-gulose, D-idose, D-galactose, D-talose, D-psicose, D-fructose, D-sorbose, or D-tagatose. In some cases, the modified hexokinase disclosed herein phosphorylates D-glucose. In some cases, the modified hexokinase disclosed herein phosphorylates L-hexose substrates.

In general, hexokinase contains four isoforms, referred to as hexokinases A, B, C, and D. Generally, hexokinases A, B, and C have high affinity toward glucose at concentrations below 1 mM and are inhibited by their products, glucose-6-phosphate. The modified hexokinase may be a modified hexokinase derived from hexokinase A, hexokinase B, hexokinase C or hexokinase D. The modified hexokinase may be a modified hexokinase derived from hexokinase B.

Hexokinase is obtained from any suitable eukaryotic or prokaryotic sources. In some cases, the hexokinase is obtained from bacteria or yeast sources. As disclosed elsewhere herein, a bacterial source is any suitable bacteria such as gram-positive bacteria or gram-negative bacteria. The gram-negative bacteria can be anaerobic, rod-shaped, or both. In some cases, the gram-negative bacterium is Escherichia coli. The bacterium may be a thermophile. The thermophile may be a hyperthermophile. Hyperthermophiles may include Thermoproteus tenax, Thermus caldophilus, Sulfolobus tokodaii, or Aeropyrum pernix. Hexokinase may be obtained from any suitable yeast such as baker's yeast, brewer's yeast, or wine yeast. Brewer's yeast may include Saccharomyces cerevisiae, Saccharomyces pastorianus (formerly known as S. carlsbergensis), Brettanomyces bruxellensis, Brettanomyces anomalus, Brettanomyces custersianus, Brettanomyces naardenensis, Brettanomyces nanus, Dekkera bruxellensis or Dekkera anomala. In some instances, the modified hexokinase is obtained from Saccharomyces cerevisiae. In some cases, the modified hexokinase B is obtained from Saccharomyces cerevisiae. In some instances, the modified hexokinase B is obtained from the hexokinase from Saccharomyces cerevisiae S288c (NP_013551.1).

In some instances, the residues involved for interaction with a ligand such as glucose or glucose-6-phosphate can include Asp205, Lys169, Asn204, Glu256 and Thr168. Sometimes, the residues involved in interaction with ATP can include Lys 111. In some instances, residues such as Asp205, Lys169, Asn204, Glu256, Thr168 and/or Lys111 can be modified to increase the hydrolysis of ATP to ADP. In additional instances, a modified hexokinase described herein can include one or more modifications at an amino acid position such as Asp205, Lys169, Asn204, Glu256, Thr168 and/or Lys111. A modified hexokinase with one or more modifications at an amino acid position such as Asp205, Lys169, Asn204, Glu256, Thr168 and/or Lys111 can further be chemically modified. The chemically modified hexokinase can be a thermostable hexokinase. The thermostable hexokinase with one or more modifications at an amino acid position such as Asp205, Lys169, Asn204, Glu256, Thr168 and/or Lys111 can also have increased ATP hydrolysis activity.

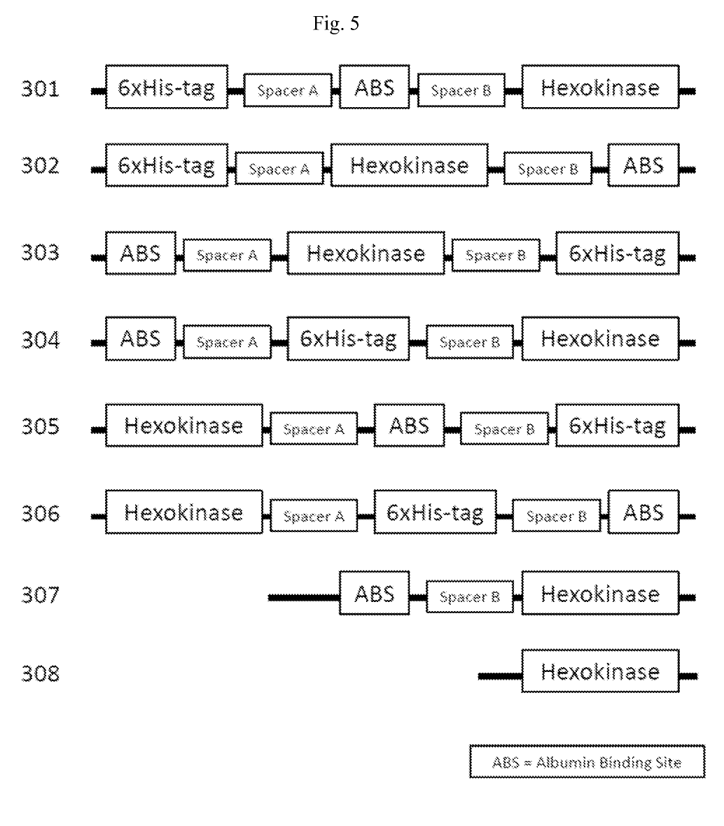

The modified hexokinase can further comprise a polyhistidine-tag (e.g., 6×His-tag) (phosphoribosyl-5-amino-1-phosphoribosyl-4-imidazolecarboxamide isomerase (his6) sequence). Sometimes, the modified hexokinase further comprises an albumin binding site. In some instances, the modified hexokinase further comprises a polyhistidine-tag (e.g., 6×His-tag) (phosphoribosyl-5-amino-1-phosphoribosyl-4-imidazolecarboxamide isomerase (his6) sequence) and an albumin binding site. The HIS (e.g., HIS(6)) tag is a purification tag comprising of six covalently linked histidine residues. The albumin binding site (ABS) is a region of a protein that is capable of recognizing and/or binding albumin. ABS may be incorporated in an immunoglobulin-binding protein. Sometimes, ABS is incorporated in Protein G such as in Streptococcal Protein G. ABS may incorporate albumin binding protein (ABP). At times, ABS is ABP, such as ABP from Streptococcal Protein G. In some instances, ABP is used as a purification tag. The HIS(6) moiety may be used as a purification tag. At times, the fusion hexokinase protein is referred to as His(6)-ABS-Hexokinase. In some instances, the His(6) tag, ABS, and Hexokinase are directly connected to each other. Alternatively, the His(6) tag, ABS, and Hexokinase are connected through spacers. Exemplary His(6)-ABS-Hexokinase constructs are shown in FIG. 5. The His(6)-ABS-Hexokinase construct may be the construct shown as 301, 302, 303, 304, 305, or 306. The His(6)-ABS-Hexokinase construct may be the construct shown as 301. In some cases, the His(6)-ABS-Hexokinase construct is further modified to remove the 6×His-tag portion, shown as 307. The 6×His-tag portion may be cleaved off during or post purification process. Spacer A may contain an enzyme cleavage site, which allows removal of the 6×His-tag from the ABS-Hexokinase portion. The His(6)-ABS-Hexokinase construct may be further modified to remove both the 6×His-tag portion and the ABS portion, shown as 308. Spacer B may also contain an enzyme cleavage site. In some cases, the enzyme cleavage sites in spacer A and spacer B are the same. In some cases, the enzyme cleavage sites in spacer A and spacer B are different.

The distance between the 6×His-tag (i.e. HIS(6)), the ABS, or both the ABS and HIS(6) and the modified hexokinase protein is defined by spacer A and spacer B. Both spacer A and spacer B represent molecule linkage such as covalent linkage. Spacer A may be at least 1, 2, 3, 4, 5, 6, 7, 8, 9, 10, or more covalently linked amino acids. Spacer B may be at least 1, 2, 3, 4, 5, 6, 7, 8, 9, 10, or more covalently linked amino acids. In some cases, the number of covalently linked amino acids in spacer A is different than the number of covalently linked amino acids in spacer B. In some instances, the number of covalently linked amino acids in spacer A is the same as the number of covalently linked amino acids in spacer B. The spacer molecule can be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or poly saccharide. In some examples, spacer A is identical to spacer B. In some instances, spacer A is different than spacer B.

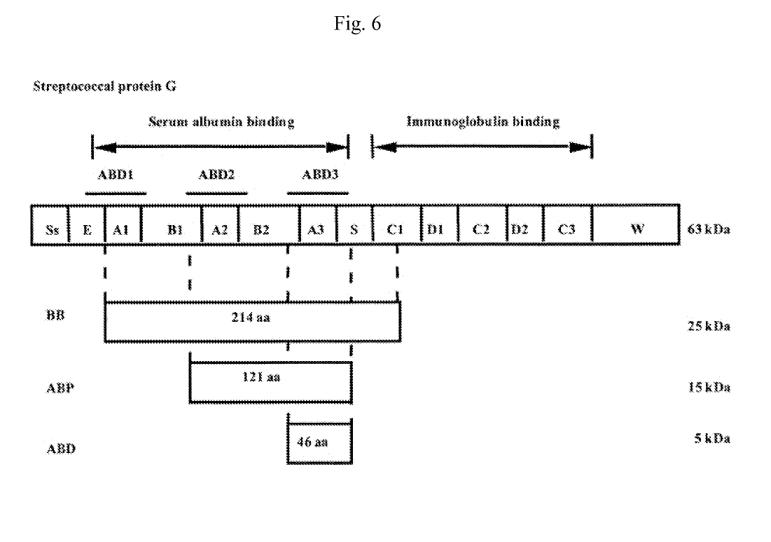

In some instances, the ABS further comprises a second binding site. The second binding site may be within the ABP. The second binding site may be linked to the ABP, either through direct covalent linkage or non-directly (e.g. a spacer). The second binding site may be at a site different from the ABP binding site and does not interfere with the interaction of ABP with albumin. The second binding site may comprise a binding site that recognizes a domain of a membrane protein. The membrane protein may be a type I membrane protein from a bacterium. The membrane protein may be a Staphylococcal protein A (SpA). The second binding site may recognize one or more domains of SpA. In some cases, the second binding site recognizes domain B of SpA. In some instances, the second binding site recognizes an analog of domain B, Z domain. In some cases, the second binding site is Z domain binding site. In some instances, the ABP further comprises the Z domain binding site, referred herein as ABP-Z. The ABS construct can be produced using the general procedure for production of the target protein construct. In some cases, the ABS construct is described in FIG. 6.

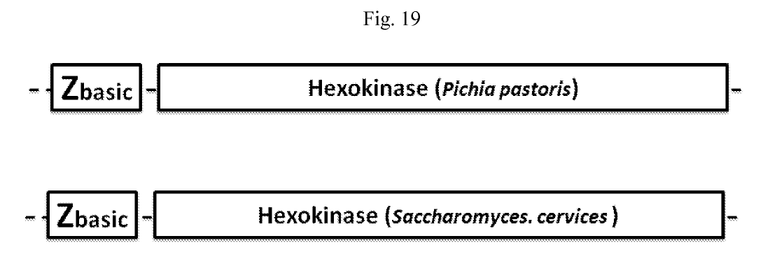

The modified hexokinase construct can comprise Z domain (or Z domain moiety) from SpA in combination with HIS, ABS, and/or biotin-tag (e.g., biotin binding domain tag). The Z domain (or Z domain moiety), HIS, ABS, and/or biotin-tag can be in any order and can be genetically introduced at the 5′ terminal of the hexokinase sequence, 3′ terminal of the hexokinase sequence, or at both termini of the hexokinase sequence. The Z domain (or Z domain moiety), HIS, ABS, and/or biotin-tag can comprise one or more spacers between each individual component. For example, a spacer can be introduced between Z domain (or Z domain moiety) and HIS, or a first spacer can be introduced between Z domain (or Z domain moiety) and HIS and a second spacer can be introduced between HIS and ABS, and so forth. Sometimes, Z domain (or Z domain moiety), HIS, ABS, and/or biotin-tag can be covalently linked without spacers between each individual component. Sometimes, hexokinase, Z domain (or Z domain moiety), HIS, ABS, and/or biotin-tag can be in any order within the modified hexokinase construct. Sometimes, Z domain (or Z domain moiety) can replace ABS in the modified hexokinase construct. The hexokinase, Z domain (or Z domain moiety), HIS, and BIO can comprise one or more spacers between each individual component and hexokinase, Z domain, HIS, and BIO can be in any order. The hexokinase, Z domain (or Z domain moiety), HIS, and BIO may not comprise one or more spacers between each individual component and hexokinase, Z domain (or Z domain moiety), HIS, and BIO can be in any order. The modified hexokinase construct may be a construct as illustrated in FIGS. 19 and 20B.

As described supra, Z domain can have the sequence as illustrated in SEQ ID NO: 7 (Z domain wild-type), SEQ ID NO: 8 (sometimes can be referred to as Zacid₂), or SEQ ID NO: 9 (sometimes can be referred to as Zbasic₂).

In some instances, the hexokinase construct is produced using the general procedure for production of the target protein construct. Sometimes, the hexokinase construct is produced using by a bioengineered host as mentioned above. As described elsewhere herein, purification of the modified hexokinase protein can utilize the general procedure for purification of the target protein construct described above, where the target protein is the modified hexokinase protein. In some cases, the purification scheme is the scheme illustrated in FIG. 2.

As disclosed above, in some instances ABS is obtained from Protein G. Protein G is an immunoglobulin-binding protein that serves as a bacterial receptor on the surface of Gram-positive bacteria. Exemplary Gram-positive bacteria include, Staphylococcus aureus, Staphylococcus epidermidis, Staphylococcus saprophyticus, Streptococcus pneumonia, Streptococcus pyogenes, Streptococcus agalactiae, Enterococcus avium, Enterococcus durans, Enterococcus faecium, Enterococcus gallinarum, Enterococcus hirae, Enterococcus solitarius, Bacillus anthracis, Bacillus oereus, Bifidobacteriu bifidum, Finegoldia magna, Lactobacillus sp. Listeria monocytogenes, Nocardia sp. Rhodococcus equi, Actinomyces sp. Clostridium botulinum, Clostridium difficile, Clostridium perfringens, Clostridum tetani, Mobiluncus sp. or Peptostreptococcus sp. In some instances, Protein G is expressed in group C and group G of Streptococcal bacteria. The various albumin binding sites in Protein G are exemplified in FIG. 6. In some instances, ABS is obtained from Streptococcal protein G (SpG), strain G148 (Olsson et al., Eur. J. Biochem. 168:319-324 (1987)). ABS may include ABP, BB, ABD, or the entire Protein G. ABS may include ABD1, ABD2, ABD3 or any combination thereof.

Any portions of the Protein G protein containing ABS may be connected to the modified hexokinase. In some cases, the ABS is ABP. In some cases ABS is at least one of ABD1, ABD2, and ABD3 regions of the Protein G. The BB region may be connected to hexokinase. The ABP region may be connected to hexokinase. The ABD region may be connected to hexokinase. The entire Streptococcal protein G may be connected to hexokinase. In some instances, the His(6) moiety is further connected to the modified hexokinase construct with ABS. The His(6) moiety may be connected to hexokinase (such construct lacks an albumin binding site).

The modified hexokinase can be generated through any suitable mutagenesis methods. The modified hexokinase can be generated through a site-directed mutagenesis method. Site-directed mutagenesis is a method that allows specific alterations or modifications within the gene of interest. The site-directed mutagenesis can utilize Cassette mutagenesis method, PCR-site-directed mutagenesis, whole plasmid mutagenesis, Kunkel's method, or in vivo site-directed mutagenesis method. The cassette mutagenesis method allows for synthesized fragments of DNA to be inserted into a plasmid using restriction enzymes and ligation methods. It does not involve polymerization. The PCR site-directed mutagenesis is similar to the cassette mutagenesis, but in which larger fragments can be obtained, separated by gel electrophoresis from the template fragments, and then ligated into the gene of interest. Whole plasmid mutagenesis, such as the Quikchange® method, allows for mutations to be inserted using one or more primers and then amplifies the entire plasmid. This method differs from the PCR site-directed mutagenesis due to the plasmid is in a linear format and that it does not need to be exponentially amplified as in a PCR. The Kunkel's method is a primer based site directed method. It differs from the previous methods in that it utilizes an E. coli strain that is deficient in dUTPase, an enzyme that prevents the bacteria from incorporating uracil during DNA replication, to distinguish between product and template strains thereby allowing for easier selection of plasmids containing the desired mutation. The in vivo site-directed mutagenesis method is further separated into the Delitto perfetto method, transplacement “pop-in pop-out” method, direct gene deletion and site-specific mutagenesis with PCR and one recyclable marker, direct gene deletion and site-specific mutagenesis with PCR and one recyclable marker using long homologous regions, and in vivo site-directed mutagenesis with synthetic oligonucleotides. They are similar to the site-directed mutagenesis methods but are under in vivo conditions.

In some instances, the modified hexokinase is generated through random mutagenesis method. Random mutagenesis is a method of generating a library of protein mutants with different functional properties. For example, random mutations are first introduced into a gene to generate a library containing thousands of different versions of this gene. Each version or variant of this gene is then expressed and the property of each expressed protein is then evaluated for function. Random mutagenesis can be achieved using error-prone PCR approach, rolling circle error-prone PCR approach, mutator strains approach, temporary mutator strains approach, insertion mutagenesis approach, ethyl methanesulfonate approach, the nitrous acid approach, or DNA shuffling. The error-prone PCR approach is a PCR method in which the polymerase has a high error rate, in some cases, up to 2%, during amplification of the wild-type sequence. In some cases, point mutations or single nucleotide mutations are the most common types of mutations in error prone PCR. Rolling circle error-prone PCR is a variant of the error-prone PCR. In this approach, the wild-type sequence is first cloned into a plasmid, and then the whole plasmid is amplified under error-prone PCR condition. Mutator strains approach utilizes a mutator strain such as XL1-Red (Strategene) which is an E. coli strain that is deficient in three of the primary DNA repair pathways (mutS, mutD and mut7) and therefore causes it to make errors during replication. The temporary mutator strains approach is similar to the mutator strain, with the exception of the E. coli strain deficient in one DNA repair pathway (mutD5), instead of three DNA repair pathways. Insertion mutagenesis approach utilizes a transposon-based system to randomly insert a 15-base sequence throughout a sequence of interest. The ethyl methanesulfonate (EMS) approach utilizes the chemical EMS to alkylate guanidine residues, thereby causing them to be incorrectly copied during DNA replication. Nitrous acid is a second chemical mutagen that introduces mutations by de-aminating adenine and cytosine residues, thereby causing transversion point mutations. DNA shuffling approach is achieved through randomly digesting the sequence of interest or a sequence library with DNAsel and then randomly re-joining the fragments using self-priming PCR. In some cases, random mutagenesis for generating a modified hexokinase is achieved using the error prone PCR approach.

In some instances, the modified hexokinase contains about 60% to about 99.9% sequence homology to the wild type hexokinase. The modified hexokinase may contain 61%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence homology to the wild type hexokinase.

The modified hexokinase may replace apyrase during the sequencing reactions disclosed herein. Apyrase is a calcium-activated nucleotide degrading enzyme that hydrolyzes ATP to AMP and inorganic phosphate. Apyrase can further act on ADP and other nucleoside triphosphates and diphosphates to generate NMP and inorganic phosphates. The use of the modified hexokinase may reduce or eliminate the use of dATPaS during sequencing reaction. The use of the modified hexokinase may improve the ability of the method in sequencing long reads. A sequencing method incorporating hexokinase or modified hexokinase is referred herein as “Lumen sequencing.”

In some instances, hexokinase as described herein is also referred to as saccharide phosphorylating enzyme. The hexokinase enzyme may be a hexokinase enzyme construct. The hexokinase construct may be a thermostable hexokinase construct. The thermostable hexokinase construct may function at a temperature above 50° C. The hexokinase may be hexokinase B.

### Luciferase

In some aspects, the invention includes a modified form of a luciferase that recognizes ATP and exhibit impaired recognition for dATP when in comparison with ATP or with no recognition of dATP. Luciferase is a class of oxidative enzymes that are capable of generating cold light, or light with no infrared or ultraviolet frequencies. In general, this chemically produced light is yellow, green, or pale red in color, with wavelengths from about 510 to 670 nanometers. Luciferase exists in numerous organisms such as fireflies, beetles, sea pansy, dinoflagellate, jellyfish, bacteria, and the marine copepod Metridia longa. The firefly luciferase can be obtained from over 2000 species of fireflies. Exemplary firefly species include species from the subfamily of Cyphonocerinae, Lampyrinae, Luciolinae, Ototetrinae, or Photurinae. Exemplary fireflies include, but are not limited to, Photinus pyralis, Luciola cruciata, Luciola italic, Luciola lateralis, Luciola mingrelica, Photuris pennsylvanica, Pyrophorus plagiophthalamus, Phrixothrix hirtus, Renilla reniformis, Gaussia princeps, Cypridina noctiluca, Cypridina hilgendorfii, Metridia longa, or Oplophorus gracilorostris. In some instances, the firefly luciferase is obtained from Photinus pyralis (AAA29795). In some cases, luciferase is obtained from a thermophile. In some cases, luciferase is obtained from Thermosynechococcus elongates.

Firefly luciferase is an enzyme of 62 kDa molecular weight. The enzyme requires ATP, molecular oxygen and the heterocyclic compound luciferin to generate light in a two-step process as shown below.
(1) Luciferin+ATP→luciferyl adenylate+PPi
(2) luciferyl adenylate+O₂→oxyluciferin+AMP+light

Luciferin further exists in two forms, D-luciferin and its enantiomer form, L-luciferin. In some cases, the substrate for the bioluminescence reaction requires D-luciferin.

In general, firefly luciferase further recognizes dATP as a substrate in addition to ATP. In sequencing methods such as pyrosequencing method, it circumvents this issue by the use of dATPaS, which serves as a substrate for polymerase but not for luciferase. Disclosed herein, is a modified luciferase that may not recognize dATP. Such modified luciferase may be obtained by random mutation. In some cases, the modified luciferase accepts ATP as the substrate, making the requirement for dATPaS unnecessary during polynucleotide sequencing.

Sometimes, the modified luciferase described herein has impaired recognition of dATP in comparison to ATP recognition. The impaired recognition of dATP can be a decrease in affinity toward dATP. The impaired recognition can be expressed as a percentage of the activity of luciferase in the presence of dATP over the activity of luciferase in the presence of ATP. In some cases, the percentage is about 0.5, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30% or higher. Sometimes, a percentage of about 2, 3, 4, 5, 6, 7, 8, 9, 10, 11, 12, 13, 14, 15, 16, 17, 18, 19, 20, 25, 30% or higher is indicative of a lack of specificity for ATP over dATP, or its inability to distinguish between dATP and ATP. Sometimes, a percentage of about 5, 6, 7, 8, 9, 10, 11, 12, 13, 14, 15, 16, 17, 18, 19, 20, 25, 30% or higher is indicative of a lack of specificity for ATP over dATP, or its inability to distinguish between dATP and ATP. In additional cases, a percentage of about 0.5, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20% or lower is indicative of a specificity toward ATP. A percentage of about 0.5, 1, 1.1, 1.2, 1.3, 1.4, 1.5, 1.6, 1.7, 1.8, 1.9, 2, 3, 4, 5% or lower may be indicative of a specificity toward ATP. Sometimes, the thermostable luciferase does not recognize dATP and recognizes ATP.

The modified luciferase described above may comprise the amino acid sequence as illustrated in SEQ ID NO: 2. The modified luciferase may further comprise a modification and/or deletion at a position within SEQ ID NO: 2, or of the N-terminus, the C-terminus, or both. The modified luciferase may comprise a modification at the N-terminus, such as for example, a modification at a position corresponding to an amino acid residue within amino acid residues 1-40, 1-30, 1-20, 1-15, 1-10, or 1-5 of SEQ ID NO: 2. The modified luciferase may further comprise a modification at one or more positions corresponding to amino acid residues S201, T202, G203, Q283, S284, S293, T214, I232, F295, E354, I423, D436, L530, or L550 of SEQ ID NO: 2. The modified luciferase may further comprise a modification at one or more positions corresponding to amino acid residues T214, I232, F295, E354, I423, D436, L530, or L550 of SEQ ID NO: 2. The modified luciferase may further comprise a modification at one or more positions corresponding to amino acid residues T214, I232, F295, I423, or L550 of SEQ ID NO: 2. The modified luciferase may further comprise a modification at one or more positions corresponding to amino acid residues T214, I232, F295, or I423 of SEQ ID NO: 2. The modified luciferase may further comprise a modification at one or more positions corresponding to amino acid residues I423, D436, or L530 of SEQ ID NO: 2.

In some instances, the modification is a mutation to a natural amino acid or an unnatural amino acid. The modification can also be a mutation to a conserved amino acid, such as for example, Phe, Tyr and Try are residues that comprise aromatic side chains and their substitution to one another can be considered conserved substitution.

The modified luciferase described herein may comprise a modification to another natural or to an unnatural amino acid at one or more positions corresponding to amino acid residues S201, T202, G203, Q283, S284, S293, T214, I232, F295, E354, I423, D436, L530, or L550 of SEQ ID NO: 2. The modification may further include T214A, I232A, F295L, E354K, I423L, D436G, L530R, or L550V. In some instances, the modified luciferase comprise a modification at one or more positions corresponding to amino acid residues T214A, I232A, F295L, E354K, I423L, D436G, L530R, or L550V of SEQ ID NO: 2. The modified luciferase may also comprise a modification at one or more positions corresponding to amino acid residues T214A, I232A, F295L, I423L, or L550V of SEQ ID NO: 2. The modified luciferase may also comprise a modification at one or more positions corresponding to amino acid residues T214A, I232A, F295L, or I423L of SEQ ID NO: 2. The modified luciferase may further comprise a modification at one or more positions corresponding to amino acid residues I423L, D436G, or L530R of SEQ ID NO: 2.

The modified luciferase may comprise a modification at I423L, D436G, and L530R of SEQ ID NO: 2. The modified luciferase may comprise a modification at T214A, I232A, F295L, and I423L of SEQ ID NO: 2. The modified luciferase may comprise a modification at T214A, I232A, F295L, I423L, and L550V of SEQ ID NO: 2. The modified luciferase comprise a modification at T214A, I232A, F295L, E354K, I423L, D436G, L530R, and L550V of SEQ ID NO: 2.

The modified luciferase comprising a modification and/or deletion at a position within SEQ ID NO: 2, or of the N-terminus, the C-terminus, or both may have an impaired recognition of dATP. The modified luciferase comprising a modification at one or more positions corresponding to amino acid residues S201, T202, G203, Q283, S284, S293, T214, I232, F295, E354, I423, D436, L530, or L550 of SEQ ID NO: 2 may have an impaired recognition of dATP.

The modified luciferase comprising a modification at one or more positions corresponding to amino acid residues T214, I232, F295, E354, I423, D436, L530, or L550 of SEQ ID NO: 2 may have an impaired recognition of dATP. The modified luciferase comprising a modification at one or more positions corresponding to amino acid residues T214, I232, F295, I423, or L550 of SEQ ID NO: 2 may have an impaired recognition of dATP. The modified luciferase comprising a modification at one or more positions corresponding to amino acid residues T214, I232, F295, or I423 of SEQ ID NO: 2 may have an impaired recognition of dATP. The modified luciferase comprising a modification at one or more positions corresponding to amino acid residues I423, D436, or L530 of SEQ ID NO: 2 may have an impaired recognition of dATP. Sometimes, the modification may include T214A, I232A, F295L, E354K, I423L, D436G, L530R, or L550V.

In some instances, the modified luciferase comprising a modification at one or more positions corresponding to amino acid residues T214A, I232A, F295L, E354K, I423L, D436G, L530R, or L550V of SEQ ID NO: 2 may have an impaired recognition of dATP. The modified luciferase comprising a modification at one or more positions corresponding to amino acid residues T214A, I232A, F295L, I423L, or L550V of SEQ ID NO: 2 may have an impaired recognition of dATP. The modified luciferase comprising a modification at one or more positions corresponding to amino acid residues T214A, I232A, F295L, or I423L of SEQ ID NO: 2 may have an impaired recognition of dATP. The modified luciferase comprising a modification at one or more positions corresponding to amino acid residues I423L, D436G, or L530R of SEQ ID NO: 2 may have an impaired recognition of dATP.

In additional cases, the modified luciferase comprising a modification at I423L, D436G, and L530R of SEQ ID NO: 2 may have an impaired recognition of dATP. The modified luciferase comprising a modification at T214A, I232A, F295L, and I423L of SEQ ID NO: 2 may have an impaired recognition of dATP. The modified luciferase comprising a modification at T214A, I232A, F295L, I423L, and L550V of SEQ ID NO: 2 may have an impaired recognition of dATP. The modified luciferase comprising a modification at T214A, I232A, F295L, E354K, I423L, D436G, L530R, and L550V of SEQ ID NO: 2 may have an impaired recognition of dATP.

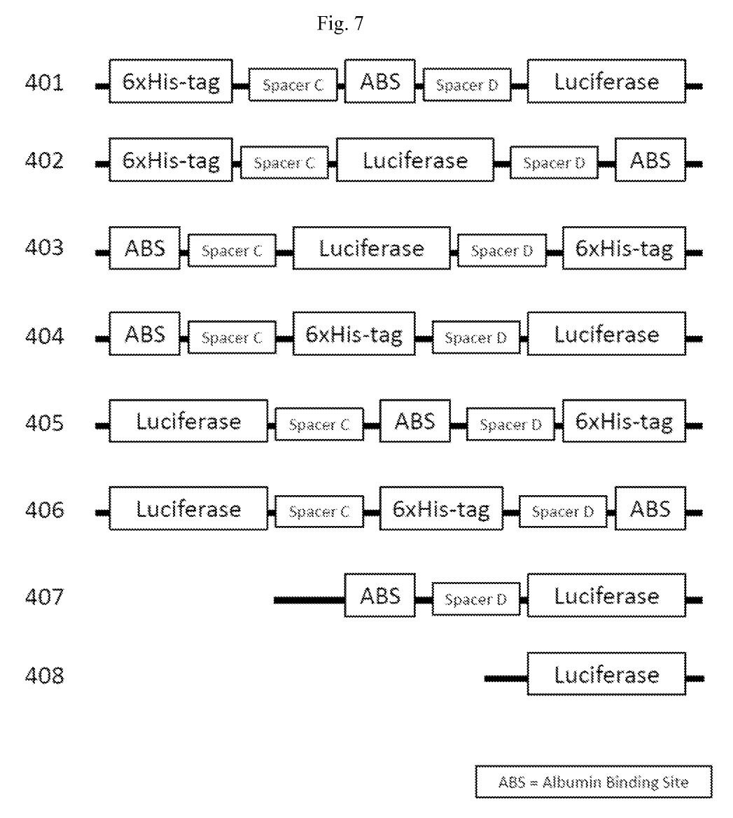

In some instances, the modified luciferase further comprises a polyhistidine-tag (6×His-tag). Sometimes, the modified luciferase further comprises an albumin binding site (ABS). In some instances, the modified luciferase further comprises a polyhistidine-tag (6×His-tag) and an albumin binding site. In some instances, ABP is used as a purification tag. Sometimes, HIS(6) (i.e. 6×his-tag) is used as a purification tag. The ABS may be albumin binding protein (ABP). See FIG. 6. ABS can be incorporated in an immunoglobulin-binding protein. Sometimes, ABS is incorporated in Protein G such as in Streptococcal Protein G. In some cases, ABS incorporates albumin binding protein (ABP). At times, ABS is ABP, such as ABP from Streptococcal Protein G. In some instances, a fusion luciferase protein is referred to as His(6)-ABS-Luciferase. The His(6) tag, ABS, and Luciferase may be connected directly to each other. In some instances, His(6) tag, ABS, and Luciferase are connected through spacers. Exemplary His(6)-ABS-Luciferase constructs are shown in FIG. 7. The His(6)-ABS-Luciferase construct may be the construct shown as 401, 402, 403, 404, 405, or 406. The His(6)-ABS-Luciferase construct may be the construct shown as 401. The His(6)-ABS-Luciferase construct may further be modified to remove the 6×His-tag portion, shown as 407. The 6×His-tag portion may be cleaved off during or post purification process. Spacer C may contain an enzyme cleavage site, which allows removal of the 6×His-tag from the ABS-Luciferase portion. The His(6)-ABS-Luciferase construct may further be modified to remove both the 6×His-tag portion and the ABS portion, shown as 408. Spacer D may also contain an enzyme cleavage site. In some cases, the enzyme cleavage sites in spacer C and spacer D are the same. Sometimes, the enzyme cleavage sites in spacer C and spacer D are different.

The distance between the either the ABS, the HIS(6) or both, and the modified luciferase protein is defined by spacer C and spacer D. Both spacer C and spacer D represent molecule linkage such as covalent linkage. Spacer C can be at least 1, 2, 3, 4, 5, 6, 7, 8, 9, 10, or more covalently linked amino acids. Spacer D can be at least 1, 2, 3, 4, 5, 6, 7, 8, 9, 10, or more covalently linked amino acids. The number of covalently linked amino acids in spacer C can be different than the number of covalently linked amino acids in spacer D. In some instances, the number of covalently linked amino acids in spacer C is the same as the number of covalently linked amino acids in spacer D. The spacer molecule can be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or poly saccharide. In some examples spacer C is identical to spacer D. In some instances, spacer C is different than spacer D.

In some instances, the ABS further comprises a second binding site. The second binding site can be within the ABP. The second binding site can be linked to the ABP, either through direct covalent linkage or non-directly (e.g. a spacer). The second binding site can be at a site different from the ABP binding site and does not interfere with the interaction of ABP with albumin. The second binding site can be a binding site that recognizes a domain of a membrane protein. The membrane protein can be a type I membrane protein from a bacterium. The membrane protein can be a Staphylococcal protein A (SpA). The second binding site can recognize one or more domains of SpA. In some cases, the second binding site recognizes domain B of SpA. In some instances, the second binding site recognizes an analog of domain B, Z domain. In some cases, the second binding site is Z domain binding site. In some instances, ABP further comprises the Z domain binding site, referred herein as ABP-Z. In some cases, the ABS construct is produced using the general procedure for production of the target protein construct. In some cases, the ABS construct is described in FIG. 6.

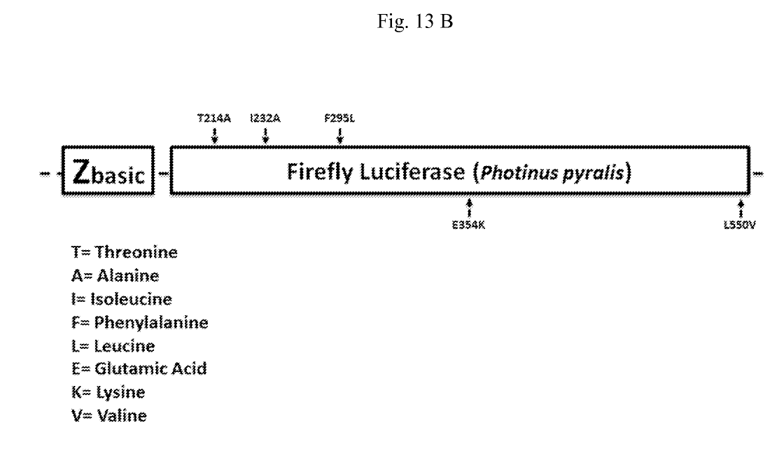

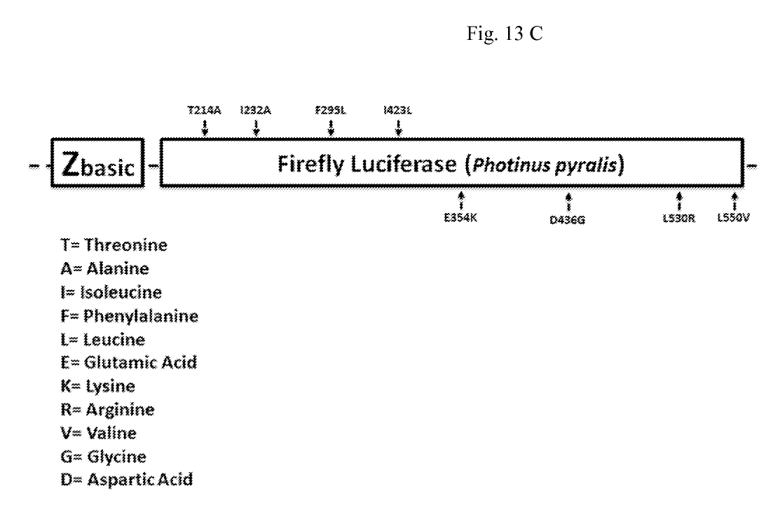

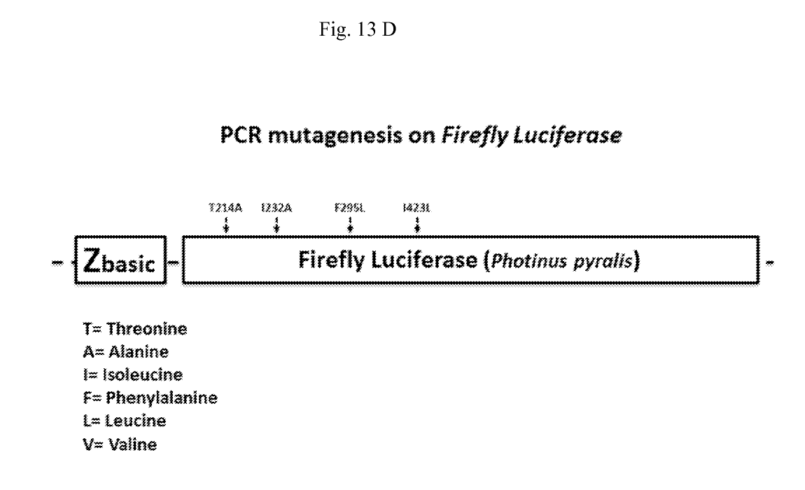

The modified luciferase construct can comprise Z domain (or Z domain moiety) from SpA in combination with HIS, ABS, and/or biotin-tag (e.g., biotin binding domain tag). The Z domain (or Z domain moiety), HIS, ABS, and/or biotin-tag can be in any order and can be genetically introduced at the 5′ terminal of the luciferase sequence, 3′ terminal of the luciferase sequence, or at both termini of the luciferase sequence. The Z domain (or Z domain moiety), HIS, ABS, and/or biotin-tag can comprise one or more spacers between each individual component. For example, a spacer can be introduced between Z domain (or Z domain moiety) and HIS, or a first spacer can be introduced between Z domain (or Z domain moiety) and HIS and a second spacer can be introduced between HIS and ABS, and so forth. Sometimes, Z domain (or Z domain moiety), HIS, ABS, and/or biotin-tag can be covalently linked without spacers between each individual component. Sometimes, hexokinase, Z domain (or Z domain moiety), HIS, ABS, and/or biotin-tag can be in any order within the modified luciferase construct. Sometimes, Z domain (or Z domain moiety) can replace ABS in the modified luciferase construct. The luciferase, Z domain (or Z domain moiety), HIS, and BIO can comprise one or more spacers between each individual component and luciferase, Z domain, HIS, and BIO can be in any order. The luciferase, Z domain (or Z domain moiety), HIS, and BIO may not comprise one or more spacers between each individual component and luciferase, Z domain (or Z domain moiety), HIS, and BIO can be in any order. Exemplary modified luciferase construct include those as illustrated in FIGS. 13 and 20B.

As described supra, Z domain can have the sequence as illustrated in SEQ ID NO: 7 (Z domain wild-type), SEQ ID NO: 8 (sometimes can be referred to as Zacid₂), or SEQ ID NO: 9 (sometimes can be referred to as Zbasic₂). Biotin-tag may have the sequence as illustrated in SEQ ID NO: 10, SEQ ID NO: 11, or SEQ ID NO: 12.

In some instances, the luciferase construct is produced using the general procedure for production of the target protein construct. Sometimes, the luciferase construct is produced using by a bioengineered host as mentioned above. As described elsewhere herein, purification of the modified luciferase protein can utilize the general procedure for purification of the target protein construct described above, where the target protein is the modified the modified luciferase protein. In some cases, the purification scheme is the scheme illustrated in FIG. 2.

In some cases, the modified luciferase is expressed in a prokaryotic expression system, an eukaryotic expression system, or in a cell-free based expression system. The modified luciferase can be expressed in a prokaryotic expression system such as in E. coli. The modified luciferase can further be expressed in an eukaryotic expression system such as for example Saccharomyces cerevisiae or Pichia pastoris.

Sometimes, the modified luciferase produced in a prokaryotic expression system such as in E. coli can be stable to intracellular proteases. The modified luciferase produced in a prokaryotic expression system such as in E. coli can be stable to intracellular proteases in comparison to a modified luciferase produced in a different expression system.

Sometimes, the yield of the modified luciferase is increased in a prokaryotic expression system such as E. coli in comparison with a different expression system. The yield can be about 1-fold, 2-fold, 3-fold, 4-fold, 5-fold, 6-fold, 7-fold, 8-fold, 9-fold, 10-fold, 12-fold, 15-fold, 20-fold, or more. Sometimes, the yield is increased at an expression temperature of between 30° C. to 37° C. The yield can be about 1-fold, 2-fold, 3-fold, 4-fold, 5-fold, 6-fold, 7-fold, 8-fold, 9-fold, 10-fold, 12-fold, 15-fold, 20-fold, or more at an expression temperature of between 30° C. to 37° C.

As described above, the modified luciferase is a luciferase comprising any combinations of mutations at amino acid residue positions T214, I232, F295, E354, I423, D436, L530, and L550 of SEQ ID NO: 2, and optionally with a chemical modification. Sometimes, the modified luciferase is a luciferase comprising any combinations of mutations at amino acid residue positions T214A, I232A, F295L, E354K, I423L, D436G, L530R, and L550V and optionally with a chemical modification. Sometimes, the modified luciferase is a luciferase comprising any combinations of mutations at amino acid residue positions T214A, I232A, F295L, I423L, and L550V and optionally with a chemical modification. The modified luciferase comprising one or more of the mutations supra can have an increased expression yield in comparison with a different expression system and/or with an increased stability to intracellular proteases.

As disclosed above, in some instances ABS is obtained from Protein G. Protein G is an immunoglobulin-binding protein that serves as a bacterial receptor on the surface of Gram-positive bacteria. In some instances, Protein G is expressed in group C and group G of Streptococcal bacteria. In some instances, ABP is obtained from Streptococcal protein G (SpG), strain G148.

Any portions of the Protein G protein containing ABS may be connected to the modified luciferase. The ABS may be ABP. ABS may be at least one of ABD1, ABD2, and ABD3 regions of the Protein G. BB region may be connected to luciferase. ABP region may be connected to luciferase. ABD region may be connected to luciferase. The entire Streptococcal protein G may be connected to luciferase. In some instances, the HIS (e.g., His(6)) moiety is further connected to the modified luciferase construct with ABS. The HIS (e.g., His(6)) moiety may be connected to luciferase (such construct lacks an albumin binding site).

The modified luciferase can be generated through any suitable mutagenesis methods. The modified luciferase may be generated through a site-directed mutagenesis method. As disclosed above, site-directed mutagenesis is a method that allows specific alterations or modifications within the gene of interest. The site-directed mutagenesis can utilize Cassette mutagenesis method, PCR-site-directed mutagenesis, whole plasmid mutagenesis, Kunkel's method, or in vivo site-directed mutagenesis method. The modified luciferase can be generated through random mutagenesis method. Random mutagenesis is a method of generating a library of protein mutants with different functional properties. Random mutagenesis can be achieved using error-prone PCR approach, rolling circle error-prone PCR approach, mutator strains approach, temporary mutator strains approach, insertion mutagenesis approach, ethyl methanesulfonate approach, the nitrous acid approach, or DNA shuffling. Random mutagenesis utilizing an error-prone PCR approach can be used to generate a modified luciferase.

In some cases, luciferase as described herein is also referred to as a bioluminescent enzyme. The luciferase may be a firefly luciferase. Sometimes, the luciferase does not recognize dATP. Occasionally, the luciferase does not recognize dATP but recognizes ATP. The luciferase may be a thermostable luciferase. The luciferase construct may be a thermostable luciferase construct. The thermostable luciferase construct may function at a temperature above 50° C.

### Thermostable Enzymes

In some aspects, the invention includes chemically modified forms of the enzymes disclosed herein, with enhanced thermal stabilities. In some cases when amplification occurs at an ambient temperature, single-stranded DNA-binding protein (SSB) is included in the reagent. This protein is able to bind to single-stranded regions of DNA, thereby preventing secondary structure formation. At an elevated temperature, DNA will not form secondary structure, therefore, making SSB requirement unnecessary. The chemical modification disclosed herein allows one or more enzymes to function at elevated temperatures.

The temperature may be about 25° C. or above. The temperature may be about 30° C., about 37° C., about 40° C. or above. The temperature may be about 45° C. or above. Sometimes, the temperature may be between about 25° C. and about 100° C., about 30° C. and about 95° C., about 37° C. and about 90° C., about 40° C. and about 85° C., or about 45° C. and about 80° C. The temperature may be between about 50° C. and about 80° C. In some instances, the temperature is between about 50.05° C. and about 80° C. Sometimes, the temperature is between about 50° C. and about 55° C. The temperature may be at least above 50.05° C. The temperature may be at least 50.06° C., 50.5° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., or above 79° C. The temperature may be at most 50.5° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79°, or less. In some instances, the temperature is about 50.5° C., about 51° C., about 52° C., about 53° C., about 54° C., or about 55° C.

In some cases, one or more of the enzymes are chemically modified to generate thermal stability. The chemically modified thermally stable enzymes may include one or more of hexokinase, luciferase or ATP sulfurylase. The chemically modified thermally stable enzymes may include one or more of modified hexokinase, modified luciferase or ATP sulfurylase. The chemically modified thermally stable enzymes may include one or more of ABS modified hexokinase, ABS modified luciferase or ATP sulfurylase. The chemically modified thermally stable enzymes may include one or more of His(6) modified hexokinase, His(6) modified luciferase or ATP sulfurylase. The chemically modified thermally stable enzymes may include one or more of His(6) and ABS modified hexokinase, Z-domain modified hexokinase, His(6) and ABS modified luciferase, Z-domain luciferase, or ATP sulfurylase. The chemically modified thermally stable enzymes may include hexokinase, and luciferase. The chemically modified thermally stable enzymes may include hexokinase, and ATP sulfurylase. The chemically modified thermally stable enzymes may include luciferase, and ATP sulfurylase. In some cases, only hexokinase is chemically modified. Sometimes, only luciferase is modified. In some instances, only ATP sulfurylase is chemically modified.

Sometimes, the modified luciferase comprising a modification and/or deletion at a position within SEQ ID NO: 2, or of the N-terminus, the C-terminus, or both may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues S201, T202, G203, Q283, S284, S293, T214, I232, F295, E354, I423, D436, L530, or L550 of SEQ ID NO: 2 may be further chemically modified to increase the luciferase's stability at a temperature such as for example between about 50° C. and about 80° C., between about 55° C. and about 70° C., or between about 60° C. and about 65° C.

The modified luciferase comprising a modification at one or more positions corresponding to amino acid residues T214, I232, F295, E354, I423, D436, L530, or L550 of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues T214, I232, F295, I423, or L550 of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues T214, I232, F295, or I423 of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues I423, D436, or L530 of SEQ ID NO: 2 may be further chemically modified to increase the luciferase's stability at a temperature such as for example between about 50° C. and about 80° C., between about 55° C. and about 70° C., or between about 60° C. and about 65° C. Sometimes, the modification may include T214A, I232A, F295L, E354K, I423L, D436G, L530R, or L550V.

In some instances, the modified luciferase comprising a modification at one or more positions corresponding to amino acid residues T214A, I232A, F295L, E354K, I423L, D436G, L530R, or L550V of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues T214A, I232A, F295L, I423L, or L550V of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues T214A, I232A, F295L, or I423L of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at one or more positions corresponding to amino acid residues I423L, D436G, or L530R of SEQ ID NO: 2 may be further chemically modified to increase the luciferase's stability at a temperature such as for example between about 50° C. and about 80° C., between about 55° C. and about 70° C., or between about 60° C. and about 65° C.

In additional cases, the modified luciferase comprising a modification at I423L, D436G, and L530R of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at T214A, I232A, F295L, and I423L of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at T214A, I232A, F295L, I423L, and L550V of SEQ ID NO: 2 may be further chemically modified to increase the luciferase's stability at a temperature such as for example between about 50° C. and about 80° C., between about 55° C. and about 70° C., or between about 60° C. and about 65° C. The modified luciferase comprising a modification at T214A, I232A, F295L, E354K, I423L, D436G, L530R, and L550V of SEQ ID NO: 2 may be further chemically modified to increase the luciferase's stability at a temperature such as for example between about 50° C. and about 80° C., between about 55° C. and about 70° C., or between about 60° C. and about 65° C.

The chemically modified luciferase comprising a modification and/or deletion at a position within SEQ ID NO: 2, or of the N-terminus, the C-terminus, or both may have an impaired recognition of dATP. The chemically modified luciferase comprising a modification at one or more positions corresponding to amino acid residues S201, T202, G203, Q283, S284, S293, T214, I232, F295, E354, I423, D436, L530, or L550 of SEQ ID NO: 2 may have an impaired recognition of dATP.

The chemically modified luciferase comprising a modification at one or more positions corresponding to amino acid residues T214, I232, F295, E354, I423, D436, L530, or L550 of SEQ ID NO: 2 may have an impaired recognition of dATP. The chemically modified luciferase comprising a modification at one or more positions corresponding to amino acid residues T214, I232, F295, I423, or L550 of SEQ ID NO: 2 may have an impaired recognition of dATP. The chemically modified luciferase comprising a modification at one or more positions corresponding to amino acid residues T214, I232, F295, or I423 of SEQ ID NO: 2 may have an impaired recognition of dATP. The chemically modified luciferase comprising a modification at one or more positions corresponding to amino acid residues I423, D436, or L530 of SEQ ID NO: 2 may have an impaired recognition of dATP. Sometimes, the modification may include T214A, I232A, F295L, E354K, I423L, D436G, L530R, or L550V.

In some instances, the chemically modified luciferase comprising a modification at one or more positions corresponding to amino acid residues T214A, I232A, F295L, E354K, I423L, D436G, L530R, or L550V of SEQ ID NO: 2 may have an impaired recognition of dATP. The chemically modified luciferase comprising a modification at one or more positions corresponding to amino acid residues T214A, I232A, F295L, I423L, or L550V of SEQ ID NO: 2 may have an impaired recognition of dATP. The chemically modified luciferase comprising a modification at one or more positions corresponding to amino acid residues T214A, I232A, F295L, or I423L of SEQ ID NO: 2 may have an impaired recognition of dATP. The chemically modified luciferase comprising a modification at one or more positions corresponding to amino acid residues I423L, D436G, or L530R of SEQ ID NO: 2 may have an impaired recognition of dATP.

In additional cases, the chemically modified luciferase comprising a modification at I423L, D436G, and L530R of SEQ ID NO: 2 may have an impaired recognition of dATP. The chemically modified luciferase comprising a modification at T214A, I232A, F295L, and I423L of SEQ ID NO: 2 may have an impaired recognition of dATP. The chemically modified luciferase comprising a modification at T214A, I232A, F295L, I423L, and L550V of SEQ ID NO: 2 may have an impaired recognition of dATP. The chemically modified luciferase comprising a modification at T214A, I232A, F295L, E354K, I423L, D436G, L530R, and L550V of SEQ ID NO: 2 may have an impaired recognition of dATP.

In some instances, the chemically modified luciferase comprising a modification and/or deletion at a position within SEQ ID NO: 2, or of the N-terminus, the C-terminus, or both may have an impaired recognition of dATP at an elevated temperature. The chemically modified luciferase comprising a modification at one or more positions corresponding to amino acid residues S201, T202, G203, Q283, S284, S293, T214, I232, F295, E354, I423, D436, L530, or L550 of SEQ ID NO: 2 may have an impaired recognition of dATP at an elevated temperature.

The chemically modified luciferase comprising a modification at one or more positions corresponding to amino acid residues T214, I232, F295, E354, I423, D436, L530, or L550 of SEQ ID NO: 2 may have an impaired recognition of dATP at an elevated temperature. The chemically modified luciferase comprising a modification at one or more positions corresponding to amino acid residues T214, I232, F295, I423, or L550 of SEQ ID NO: 2 may have an impaired recognition of dATP at an elevated temperature. The chemically modified luciferase comprising a modification at one or more positions corresponding to amino acid residues T214, I232, F295, or I423 of SEQ ID NO: 2 may have an impaired recognition of dATP at an elevated temperature. The chemically modified luciferase comprising a modification at one or more positions corresponding to amino acid residues I423, D436, or L530 of SEQ ID NO: 2 may have an impaired recognition of dATP at an elevated temperature. Sometimes, the modification may include T214A, I232A, F295L, E354K, I423L, D436G, L530R, or L550V.

In some instances, the chemically modified luciferase comprising a modification at one or more positions corresponding to amino acid residues T214A, I232A, F295L, E354K, I423L, D436G, L530R, or L550V of SEQ ID NO: 2 may have an impaired recognition of dATP at an elevated temperature. The chemically modified luciferase comprising a modification at one or more positions corresponding to amino acid residues T214A, I232A, F295L, I423L, or L550V of SEQ ID NO: 2 may have an impaired recognition of dATP at an elevated temperature. The chemically modified luciferase comprising a modification at one or more positions corresponding to amino acid residues T214A, I232A, F295L, or I423L of SEQ ID NO: 2 may have an impaired recognition of dATP at an elevated temperature. The chemically modified luciferase comprising a modification at one or more positions corresponding to amino acid residues I423L, D436G, or L530R of SEQ ID NO: 2 may have an impaired recognition of dATP at an elevated temperature.

In additional cases, the chemically modified luciferase comprising a modification at I423L, D436G, and L530R of SEQ ID NO: 2 may have an impaired recognition of dATP at an elevated temperature. The chemically modified luciferase comprising a modification at T214A, I232A, F295L, and I423L of SEQ ID NO: 2 may have an impaired recognition of dATP at an elevated temperature. The chemically modified luciferase comprising a modification at T214A, I232A, F295L, I423L, and L550V of SEQ ID NO: 2 may have an impaired recognition of dATP at an elevated temperature. The chemically modified luciferase comprising a modification at T214A, I232A, F295L, E354K, I423L, D436G, L530R, and L550V of SEQ ID NO: 2 may have an impaired recognition of dATP at an elevated temperature.

The elevated temperature may be between about 50° C. and about 80° C. The elevated temperature may be between about 50.05° C. and about 80° C. Sometimes, the elevated temperature is between about 50° C. and about 55° C. The elevated temperature may be at least above 50.05° C. The elevated temperature may be at least 50.06° C., 50.5° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., or above 79° C. The elevated temperature may be at most 50.5° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79°, or less. In some instances, the elevated temperature is about 50.5° C., about 51° C., about 52° C., about 53° C., about 54° C., or about 55° C.

In some instances, the chemically modified luciferase comprising a modification and/or deletion at a position within SEQ ID NO: 2, or of the N-terminus, the C-terminus, or both may have an impaired recognition of dATP with an increase in temperature. The chemically modified luciferase comprising a modification at one or more positions corresponding to amino acid residues S201, T202, G203, Q283, S284, S293, T214, I232, F295, E354, I423, D436, L530, or L550 of SEQ ID NO: 2 may have an impaired recognition of dATP with an increase in temperature.

The chemically modified luciferase comprising a modification at one or more positions corresponding to amino acid residues T214, I232, F295, E354, I423, D436, L530, or L550 of SEQ ID NO: 2 may have an impaired recognition of dATP with an increase in temperature. The chemically modified luciferase comprising a modification at one or more positions corresponding to amino acid residues T214, I232, F295, I423, or L550 of SEQ ID NO: 2 may have an impaired recognition of dATP with an increase in temperature. The chemically modified luciferase comprising a modification at one or more positions corresponding to amino acid residues T214, I232, F295, or I423 of SEQ ID NO: 2 may have an impaired recognition of dATP with an increase in temperature. The chemically modified luciferase comprising a modification at one or more positions corresponding to amino acid residues I423, D436, or L530 of SEQ ID NO: 2 may have an impaired recognition of dATP with an increase in temperature. Sometimes, the modification may include T214A, I232A, F295L, E354K, I423L, D436G, L530R, or L550V.

In some instances, the chemically modified luciferase comprising a modification at one or more positions corresponding to amino acid residues T214A, I232A, F295L, E354K, I423L, D436G, L530R, or L550V of SEQ ID NO: 2 may have an impaired recognition of dATP with an increase in temperature. The chemically modified luciferase comprising a modification at one or more positions corresponding to amino acid residues T214A, I232A, F295L, I423L, or L550V of SEQ ID NO: 2 may have an impaired recognition of dATP with an increase in temperature. The chemically modified luciferase comprising a modification at one or more positions corresponding to amino acid residues T214A, I232A, F295L, or I423L of SEQ ID NO: 2 may have an impaired recognition of dATP with an increase in temperature. The chemically modified luciferase comprising a modification at one or more positions corresponding to amino acid residues I423L, D436G, or L530R of SEQ ID NO: 2 may have an impaired recognition of dATP with an increase in temperature.

In additional cases, the chemically modified luciferase comprising a modification at I423L, D436G, and L530R of SEQ ID NO: 2 may have an impaired recognition of dATP with an increase in temperature. The chemically modified luciferase comprising a modification at T214A, I232A, F295L, and I423L of SEQ ID NO: 2 may have an impaired recognition of dATP with an increase in temperature. The chemically modified luciferase comprising a modification at T214A, I232A, F295L, I423L, and L550V of SEQ ID NO: 2 may have an impaired recognition of dATP with an increase in temperature. The chemically modified luciferase comprising a modification at T214A, I232A, F295L, E354K, I423L, D436G, L530R, and L550V of SEQ ID NO: 2 may have an impaired recognition of dATP with an increase in temperature.

ATP sulfurylase may be obtained from any suitable prokaryotic or eukaryotic sources. In some cases, ATP sulfurylase is obtained from Saccharomyces cerevisiae, Penicillium chrysogenum, rat liver, and plants. ATP sulfurylase genes may be cloned from prokaryotes (see e.g., Leyh, et al., 1992. J. Biol. Chem. 267: 10405-10410; Schwedock and Long, 1989. Mol. Plant Microbe Interaction 2: 181-194; Laue and Nelson, 1994. J. Bacteriol. 176: 3723-3729); eukaryotes (see e.g., Cherest, et al., 1987. Mol. Gen. Genet. 210: 307-313; Mountain and Korch, 1991. Yeast 7: 873-880; Foster, et al., 1994. J. Biol. Chem. 269: 19777-19786); plants (see e.g., Leustek, et al., 1994. Plant Physiol. 105: 897-90216); and animals (see e.g., Li, et al., 1995. J. Biol. Chem. 270: 29453-29459). The enzyme is a homo-oligomer or heterodimer, depending upon the specific source (see e.g., Leyh and Suo, 1992. J. Biol. Chem. 267: 542-ATP sulfurylase may be obtained from thermophiles. ATP sulfurylase may be obtained from Archaeoglobus or Pyrococcus spp. ATP sulfurylase may be obtained from Bacillus steareothermophilus.

The invention disclosed herein can utilize one or more of the chemically modified thermally stable enzymes under elevated temperatures. The invention disclosed herein can utilize chemically modified thermally stable hexokinase, luciferase, and ATP sulfurylase. The invention disclosed herein can utilize chemically modified thermally stable hexokinase and luciferase along with a thermally stable ATP sulfurylase without chemical modifications. An alternative ATP converting enzymes can be used in place of ATP sulfurylase. The additional ATP converting enzymes can include pyruvate orthophosphate dikinase. The invention disclosed herein can utilize chemically modified thermally stable hexokinase and luciferase along with unmodified pyruvate orthophosphate dikinase. The pyruvate orthophosphate dikinase can be chemically modified to be thermally stable. The invention disclosed herein can utilize chemically modified thermally stable hexokinase and luciferase along with a thermally stable pyruvate orthophosphate dikinase. The pyruvate orthophosphate dikinase can be from a thermophilic bacterium. The invention disclosed herein can utilize chemically modified thermally stable hexokinase and luciferase along with a thermophilic pyruvate orthophosphate dikinase.

The invention disclosed herein can utilize chemically modified thermally stable hexokinase and ATP sulfurylase, with a luciferase that was not chemically modified. The luciferase can be obtained from a thermophilic bacterium. The invention disclosed herein can utilize chemically modified thermally stable hexokinase and ATP sulfurylase, with a thermophilic luciferase. The thermophilic luciferase can be a modified luciferase that recognizes ATP but not dATP. The invention disclosed herein can utilize chemically modified thermally stable hexokinase and ATP sulfurylase, with a thermophilic modified luciferase that recognizes ATP but not dATP.

The chemically modified hexokinase can be the modified hexokinase comprising of the His(6)-ABS-Hexokinase protein construct. In some cases, both the ABS and the hexokinase portion of the protein are chemically modified. Sometimes, only the ABS portion of the protein is chemically modified. In some cases, only the hexokinase portion of the protein is chemically modified. The various version of the chemically modified thermally stable His(6)-ABS-Hexokinase protein can be used in the above described permutations of the invention.

Alternatively, hexokinase can be substituted with apyrase. The invention disclosed herein can utilize chemically modified thermally stable luciferase, and ATP sulfurylase with either an apyrase that was not chemically modified, or a chemically modified apyrase. The apyrase that was not chemically modified can be a thermophilic or thermally stable apyrase without the additional chemical modification disclosed herein. The invention disclosed herein can utilize either a chemically modified thermally stable luciferase, or chemically modified thermally stable ATP sulfurylase with either an unmodified apyrase, or a chemically modified apyrase. The invention disclosed herein can utilize chemically modified thermally stable luciferase, an unmodified pyruvate orthophosphate dikinase or a chemically modified pyruvate orthophosphate dikinase, and either a chemically modified apyrase or an apyrase that was not chemically modified.

Alternatively, a number of thermostable DNA polymerases are available which may also be used in the invention and with the described permutations above. Exemplary thermostable DNA polymerases include, but are not limited to, Taq, Phusion® DNA polymerase, Q5® High Fidelity DNA Polymerase, LongAmp® DNA polymerase, Expand High Fidelity polymerase, HotTub polymerase, Pwo polymerase, Tfl polymerase, Tli polymerase, UlTma polymerase, Pfu polymerase, Vent polymerase, or Deep Vent polymerase.

The chemical modification of the enzyme to be modified includes reacting the enzyme with an organic aldehyde, halo-formate, anhydride, a carboxylic acid or a combination thereof. The halo format may be fluoro-formate, chloro-formate, bromo-formate, iodo-formate or astatino-formate. In some instances, the chemical modification comprises reaching the enzyme with an aldehyde. Sometimes, the chemical modification comprises reacting the enzyme with an anhydride. The anhydride may be acetic anhydride, maleic anhydride citroconic anhydride, succinic anhydride, glutaric anhydride, Diphenic anhydride, dicarboxylic anhydride, naphthalic anhydride, phthalic anhydride, or any other anhydride known in the art. Sometimes, the anhydride may be pyromellitic dianhydride or naphthalenetetracarboxylic dianhydride. In some instances, the chemical reaction may be acetylation. Sometimes, the chemical reaction may be citraconylation.

At times, the chemical reactions modified one hydrogen of the epsilon amine of at least one lysine in the protein sequence of the enzyme. Occasionally, the chemical reactions modified both hydrogens of the epsilon amine of at least one lysine in the protein sequence of the enzyme. The chemical reaction may neutralize or reverse the charge of the side chain of the modified lysine.

At least one of the epsilon amine of at least one lysine in the protein sequence of a chemically modified enzyme can be substituted with an organic moiety that includes a hydrocarbon. The hydrocarbon may be aliphatic, aromatic, comprises a double bond, or any combination thereof. The aliphatic hydrocarbon may be branched or linear. The aliphatic hydrocarbon may be a methyl, ethyl, propyl, butyl, pentyl, hexyl, heptyl, nonyl, octyl, or any aliphatic hydrocarbon of the general form CₙH₂ₙ₊₁ (n=1, 2, 3, 4, 5, 6, 7, 8, 9, 10, 11, 12, 13, 14, 15, 16, 17, 18, 19, 20 or more). The hydrocarbon may be a methyl. The hydrocarbon may incorporate a methyl and a double bond. The hydrocarbon may incorporate a carboxylic acid and a double bond. The hydrocarbon may incorporate a methyl, a carboxylic acid and a double bond. The hydrocarbon may incorporate a carboxylic ester and a double bond. The hydrocarbon may incorporate a methyl, a carboxylic ester and a double bond. The hydrocarbon may incorporate a methyl, a carboxylic acid and a double bond where the carboxylic acid and the methyl are each directly connected to the double bond. The hydrocarbon may incorporate a methyl, a carboxylic acid and a double bond where the carboxylic acid and the methyl are each directly connected to the double bond and are cis to each other. The hydrocarbon may incorporate a methyl, a carboxylic acid and a double bond where the carboxylic acid and the methyl are each directly connected to the double bond and are trans to each other. The basic side chains can be lysine side chains. The organic moiety can be the organic moiety depicted in scheme 5. The organic moiety can be the organic moiety depicted in scheme 6A, scheme 6B, or a combination of both scheme 6A and scheme 6B.

The chemical modification of the disclosed enzymes can be accomplished by acetylation or citraconylation of their reactive lysine residues (see schemes 1-4). There are 39, 22 and 39 lysine residues in firefly luciferase, ATP sulfurylase and hexokinase, respectively. All of the lysine residues in the firefly luciferase, ATP sulfurylase, and hexokinase can be modified. Sometimes, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 lysine residues in firefly luciferase are modified. In some cases, at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or less lysine residues in firefly luciferase are modified. The firefly luciferase can be modified by acetylation, by citraconylation, or by, a combination of acetylation and citraconylation methods.

In some cases, at least 1, 2, 3, 4, 5, 6, 7, 8, 9, 10, 11, 12, 13, 14, 15, 16, 17, 18, 19, 20, 21, or 22 lysine residues in ATP sulfurylase are modified. In some cases, at most 2, 3, 4, 5, 6, 7, 8, 9, 10, 11, 12, 13, 14, 15, 16, 17, 18, 19, 20, 21, 22, or less lysine residues in ATP sulfurylase are modified. The ATP sulfurylase can be modified by acetylation, by citraconylation, or by a combination of acetylation and citraconylation methods.

In some cases, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 lysine residues in hexokinase are modified. In some cases, at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or less lysine residues in hexokinase are modified. The hexokinase can be modified by acetylation, by citraconylation, or by a combination of acetylation and citraconylation methods.

Modifiers can be added in the reaction to prevent some of the lysine residues from being modified. In some cases, at least 0.5, 1, 1.5, 2, 2.5, 3, 3.5, 4 millimolar (mM) or more ATP/APS are added into the reaction to prevent some of the lysine residues from being modified. Sometimes, at most 1, 1.5, 2, 2.5, 3, 3.5, 4 millimolar (mM) or less ATP/APS are added into the reaction to prevent some of the lysine residues from being modified. In some instances, at least 0.5, 1, 1.5, 2, 2.5, 3, 3.5, 4 millimolar (mM) or more ATP/APS are added into the reaction to prevent modification of active site lysine residues. In some cases, at most 1, 1.5, 2, 2.5, 3, 3.5, 4 millimolar (mM), or less ATP/APS are added into the reaction to prevent modification of active site lysine residues.

Chemical Modification of Enzymes to Render them Thermostable

In some examples, at least one of ATP sulfurylase, modified luciferase and modified hexokinase is rendered thermostable through chemical reaction. The chemical reaction can be carried on the entire enzyme. In some instances, the chemical reaction is carried on a part of the enzyme. The chemical reaction can be complete or partial. In some examples, the chemical reaction neutralizes the positively charged amino acids. Sometimes, the chemical reaction reverses the positively charged amino acids to negatively charged modified amino acids. Positively charged amino acids may include lysine, histidine or arginine. The modification may be via ion bonded species, polar bonded species, hydrogen bonded species, or modification utilizing covalent bonding. In some instances, the modification involves complexation with a neutralizing or negatively charged species such as acids. Negatively charged amino acids may include aspartic acid or glutamic acid. In some instances, the covalent bonding involves covalently binding ethers, alcohols, esters or acids to at least one of the distal amines in the side chain of the positively charged amino acid. Exemplary modification schemes shown below are for illustrative purposes only and should not be construed as limiting in any manner.

In some instances, the modification is the modification as shown in scheme 1, in which

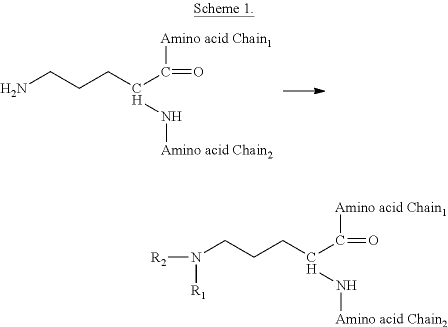

R1, R2 may be hydrogen, aliphatic (linear or branched), aromatic or conjugated. R1 and R2 may comprise one or more acid, ester, alcohol or ether groups. Acids may be carboxylic, thiocarboxylic, sulfonic or phosphonic acids. R1 and R2 may be identical or different. Amino chain1 and amino chain2 designate two different parts of the same peptide (i.e. enzyme) of which the exemplified amino acid is a part of.

The modification may be the modification as shown in scheme 2, in which

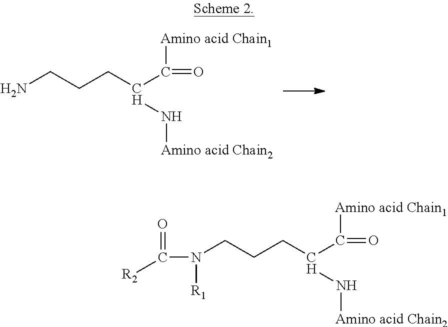

R1, R2 may be hydrogen, aliphatic (linear or branched), aromatic or conjugated. R1 and R2 may comprise one or more acid, ester, alcohol or ether groups. Acids may be carboxylic, thiocarboxylic, sulfonic or phosphonic acids. R1 and R2 may be identical or different.

The modification may be the modification as shown in scheme 3, in which

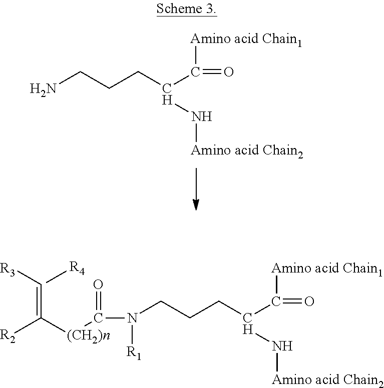

R2 R3, R4 may be hydrogen, aliphatic (linear or branched), aromatic or conjugated. R1 and R2 may comprise one or more acid, ester, alcohol or ether groups. Acids may be carboxylic, thiocarboxylic, sulfonic or phosphonic acids. R1 and R2 may be identical or different. R1 may be hydrogen or aliphatic (linear or branched), and may be 0, 1, 2, 3, 4, 5, 6, 7, 8, 9, 10 or more.

The modification may be the modification as shown in scheme 4, in which

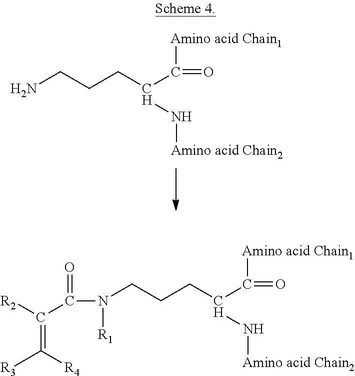

R4 may comprise a carboxylic, thiocarboxylic, sulfonic or phosphonic acid. R1, R2 and R3 may be identical or different. R1, R2 or R3 may be hydrogen or aliphatic (linear or branched). n may be 0, 1, 2, 3, 4, 5, 6, 7, 8, 9, 10 or more. In one example, R1 is hydrogen, R2 is different than R3, one of R2 and R3 is hydrogen. One of R2 and R3 is an aliphatic compound CₘH₂ₘ₊₁, where m may be 1, 2, 3, 4, 5, 6, 7, 8, 9, 10 or more.

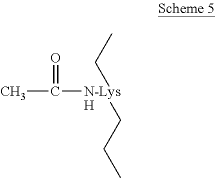

Scheme 5 depicts one specific example of a modified lysine residue after reaction with acetic anhydride.

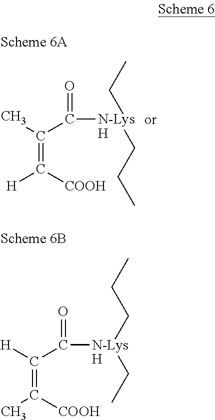

Scheme 6 depicts two specific examples (scheme 6A and scheme 6B) of a modified forms of a lysine residue after reaction with citroconic anhydride.

### Taq Polymerase

In some aspects, the invention includes an amplification enzyme for use in a method disclosed herein. The amplification enzyme can be a polymerase. The polymerase can be a Taq polymerase. The Taq polymerase can be a native Taq polymerase, or a modified Taq polymerase. As used herein, Taq polymerase refers to any Taq polymerase, (native Taq polymerase or modified Taq polymerase). The Taq polymerase can be a Taq polymerase that is free from contamination (e.g. polynucleotide contamination), which is fabricated by custom engineering of Taq polymerase and producing in a cell. The custom engineering may include custom engineering of the genetic sequence encoding the custom engineered Taq polymerase protein, custom engineering the Taq polymerase protein, or a combination thereof. The cell (i.e. host cell) may include any suitable cell such as naturally derived cell or a genetically modified cell. The host cell may be a eukaryotic cell or a prokaryotic cell. An eukaryotic cell may include fungi, animal cell or plant cell. In some instances, the eukaryotic cell includes yeast. Sometimes, the yeast is a yeast capable of digesting polysaccharides into carbon dioxide and ethanol. Sometimes the yeast is baker's yeast or brewer's yeast or wine yeast (e.g. Zygosaccharomyces or Brettanomyces). Brewer's yeast may include Saccharomyces cerevisiae, Saccharomyces pastorianus (formerly known as S. carlsbergensis), Brettanomyces bruxellensis, Brettanomyces anomalus, Brettanomyces custersianus, Brettanomyces naardenensis, Brettanomyces nanus, Dekkera bruxellensis or Dekkera anomala. Other yeast species are delineated below. The prokaryotic cell can be bacterial cell. A bacterial cell may be a gram-positive bacterium or a gram-negative bacterium. Sometimes the gram-negative bacteria is anaerobic, rod-shaped, or both. In some instances, the gram-negative bacterium is Escherichia coli (i.e. E. coli). Animal cells may include a cell from a vertebrate or from an invertebrate. An animal cell may include a cell from a marine invertebrate, fish, insects, amphibian, reptile, or mammal.

The gram-positive bacteria may be Actinobacteria, Firmicutes or Tenericutes. The gram-negative bacteria may be Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bactereia may be Acidobacteria, Chloroflexi, Chrysiogenetes, Cyanobacteria, Deferribacteres, Dictyoglomi, Thermodesulfobacteria or Thermotogae. A bacterial cell bacterial may be Escherichia coli, Clostridium botulinum or Coli bacilli.

Fungi include ascomycetes such as yeast, mold, filamentous fungi, basidiomycetes, or zygomycetes. Yeast may include Ascomycota or Basidiomycota. Ascomycota may include Saccharomycotina (true yeasts, e.g. Saccharomyces cerevisiae (baker's yeast)) or Taphrinomycotina (e.g. Schizosaccharomycetes (fission yeasts)). Basidiomycota may include Agaricomycotina (e.g. Tremellomycetes) or Pucciniomycotina (e.g. Microbotryomycetes).

Yeast or filamentous fungi may include Saccharomyces, chizosaccharomyces, Candida, Pichia, Hansenula, Kluyveromyces, Zygosaccharomyces, Yarrowia, Trichosporon, Rhodosporidi, Aspergillus, Fusarium, or Trichoderma. The Yeast or filamentous fungi may include Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorphs, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, or Trichoderma reesei. Yeast may also include Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii or Saccharomyces boulardii. Sometimes, the yeast is pichia pastoris. Sometimes, the yeast is saccharomyces cerevisiae. Sometimes, the yeast is saccharomyces cerevisiae S288c (NP_013551.1).

Cells may be of a mollusk, arthropod, annelid or sponge. The mammalian cell may be of a primate, ape, equine, bovine, porcine, canine, feline or rodent. The mammal may be a primate, ape, dog, cat, rabbit or ferret. The rodent may be a mouse, rat, hamster, gerbil, hamster, chinchilla, fancy rat, or guinea pig. The bird cell may be of a canary, parakeet or parrots. The reptile cell may be of a turtles, lizard or snake. The fish cell may of a tropical fish. The fish cell may be of a zebrafish (e.g. Danio rerio). In some instances the cell may be of a nematode (e.g. C. elegans). The amphibian cell may be of a frog. The arthropod cell may be of a tarantula or hermit crab.

Cells may be derived from knock-out or knock-in versions of the aforementioned species may also be used. Engineering may include the use of genetic vectors such as PIC-9. The vectors may comprise one or more polynucleotide that encodes for at least the following two proteins: DNA polymerase and albumin. DNA polymerase may include the polymerase from Thermus aquaticus (Taq polymerase), Terminal deoxynucleotidyl transferase (TdT) (also known as DNA nucleotidylexotransferase (DNTT) or terminal transferase), Reverse transcriptase (RT), or any other polynucleotide polymerase known in the art. Additional polymerases include, but are not limited to, Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Taq® DNA polymerase, One Taq® Hot Start DNA polymerase, phi29 DNA polymerase, Phusion® Hot Start Flex DNA polymerase, Phusion® High-Fidelity DNA polymerase, Q5®+Q5® Hot Start DNA polymerase, Sulfolobus DNA polymerase IV, T4 DNA polymerase, Theminator™ DNA polymerase, Vent_{R}™ DNA polymerase, and Vent_{R}™ (exo-) DNA polymerase.

DNA polymerase may be of the genus Thermus, Bacillus, Thermococcus, Pyrococcus, Aeropyrum, Aquifex, Sulfolobus, Pyrolobus, or Methanopyrus. DNA polymerase may include the polymerase from the species Thermus aquatics, Thermus thermophilus, Bacillus stearothermophilus, Aquifex pyrophilus, Geothermobacterium ferrireducens, Thermotoga maritime, Thermotoga neopolitana, Thermotoga petrophila, Thermotoga naphthophila, Acidianus infernus, Aeropyrum pernix, Archaeoglobus fulgidus, Archaeoglobus profundus, Caldivirga maquilingensis, Desulfurococcus amylolyticus, Desulfurococcus mobilis, Desulfurococcus mucosus, Ferroglobus placidus, Geoglobus ahangari, Hyperthermus butylicus, Ignicoccus islandicus, Ignicoccus pacificus, Methanococcus jannaschii, Methanococcus fervens, Methanococcus igneus, Methanococcus infernus, Methanopyrus kandleri, Methanothermus fervidus, Methanothermus sociabilis, Palaeococcus ferrophilus, Pyrobaculum aerophilum, Pyrobaculum calidifontis, Pyrobaculum islandicum, Pyrobaculum oguniense, Pyrococcus furiosus, Pyrococcus abyssi, Pyrococcus horikoshii, Pyrococcus woesei, Pyrodictium abyssi, Pyrodictium brockii, Pyrodictium occultism, Pyrolobus fumarii, Staphylothermus marinus, Stetteria hydrogenophila, Sulfolobus solfataricus, Sulfolobus shibatae, Sulfolobus tokodaii, Sulfophobococcus zilligii, Sulfurisphaera ohwakuensis, Thermococcus kodakaraensis, Thermococcus celer, Thermococcus litoralis, Thermodiscus maritimus, Thermofilum pendens, Thermoproteus tenax, Thermoproteus neutrophilus, Thermosphaera aggregans, Vulcanisaeta distributa, or Vulcanisaeta souniana.

Albumin may include native or genetically modified albumin. Albumin may include serum albumin. Serum albumin protein may include mammalian serum albumin. Mammalian serum albumin may include human serum albumin or bovine serum albumin.

Vectors may include any suitable vectors derived from either an eukaryotic or prokaryotic sources. Vectors may be from bacteria (e.g. E. coli), insects, yeast (e.g. Pichia pastoris), or mammalian source. Bacterial vectors may include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2. In some instances the vector is pET21 from E. coli. Insect vector may include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2. Yeast vectors may include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector. In some examples, the vector is pPIC9 from Pichia pastoris. Mammalian vectors may include transient expression vectors or stable expression vectors. Mammalian transient expression vectors may include p3×FLAG-CMV 8, pFLAG-Myc-CMV 19, pFLAG-Myc-CMV 23, pFLAG-CMV 2, pFLAG-CMV 6a, b, c, pFLAG-CMV 5.1, pFLAG-CMV 5a, b, c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some examples, the vector construct is engineered such that when the sequence carried by the vector is expressed into a protein, the protein corresponding to a genetic code of a protein able to bind albumin (e.g. serum albumin) and the protein corresponding to the polymerase (e.g. Taq polymerase) is covalently linked. The produced albumin modified polymerase may result, among others, in amplifying a polynucleotide without using anti polymerase antibody, while obtaining a clean amplification product. The protein able to bind albumin may be derived from Streptococcal Protein G. The protein binding albumin can incorporate at least one of ABD1, ABD2 and ABD3 domains. The albumin may be serum albumin. The polymerase may be Taq-polymerase.

In some examples, the covalent linkage between the protein able to bind albumin and the protein polymerase (e.g. Taq-polymerase) will allow the polymerase to be modified with the protein able to bind albumin at the n-terminus of the Taq polymerase. In some instances, the covalent linkage will allow the protein polymerase to be modified with the albumin at the N-terminus of the albumin. Sometimes, the covalent linkage between the polymerase and the protein able to bind albumin is through a direct covalent linkage. Occasionally, the covalent linkage between the polymerase and the protein able to bind albumin is through a spacer molecule linkage. The spacer molecule may be at least 1, 2, 3, 4, 5, 6, 7, 8, 9, 10 or more covalently linked amino acids. The spacer molecule may be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or poly saccharide. The polymerase may be Taq-polymerase. The albumin may be serum albumin or human serum albumin. At times, the albumin is bovine serum albumin.

The invention describes method for the production of polymerase (e.g. Taq-polymerase) in host cells. These methods may comprise modified or engineered Taq-polymerase and anti Taq polymerase in any of the above-mentioned host cells. In some examples, the invention describes method for the production of Taq polymerase in host cells (e.g. yeast or bacteria). These methods may comprise modified or engineered Taq polymerase and anti Taq polymerase in host cells.

The invention may comprise methods for high throughput protein production in a small, large or medium scale. These methods may comprise production of multiple proteins simultaneously in any of the abovementioned host cells. In some examples, the methods may comprise production of multiple proteins simultaneously in yeast or bacteria. Sometimes, the yeast is Pichia pastoris. Sometimes, the yeast is Saccharomyces cerevisiae. Sometimes, the yeast is Saccharomyces cerevisiae S288c (NP_013551.1). At times, the bacterium is Escherichia coli.

The modified polymerase (e.g. Taq-polymerase) construct incorporating the protein able to bind albumin may have a higher processivity than a polymerases without a protein able to bind albumin. As used herein, processivity is the average number of nucleotides added by the polymerase prior to the polymerase's dissociation from the DNA. The Taq polymerase construct incorporating the protein able to bind albumin may have a processivity rate of at least 1×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, or higher compared to non-albumin modified polymerases.

The polymerase (e.g. Taq-polymerase) construct incorporating the protein able to bind albumin may have a faster extension rate than polymerases that does not comprise a protein able to bind albumin. As used herein, extension rate is the maximum number of nucleotides polymerized per second per molecule of polymerase (e.g. DNA polymerase). The polymerase (e.g. Taq-polymerase) construct incorporating a protein sequence able to bind albumin may have an extension rate of at least 10, 11, 12, 13, 14, 15, 16, 17, 18, 19, 20, 25, 30, 35, 40, 45, 50, 55, 60, 90, 120 sec/kilobase, or more.

The polymerase (e.g. Taq-polymerase) construct that incorporates a protein sequence able to bind albumin may have higher fidelity than non-albumin modified polymerases. As used herein, fidelity is the ability of the polymerase to faithfully replicate a DNA molecule. Fidelity may be described by the rate of error. The Taq polymerase construct incorporating a protein sequence able to bind albumin may have an error rate of at most 1×10⁻³, 5×10⁻⁴, 1×10⁻⁴, 5×10⁻⁵, 1×10⁻⁵, 5×10⁻⁶, or less. The Taq polymerase construct incorporating a protein sequence able to bind albumin may have an error rate of at least 1×10⁻³, 5×10⁻⁴, 1×10⁻⁴, 5×10⁻⁵, 1×10⁻⁵, 5×10⁻⁶, or more.

In some instances, the invention comprises Taq polymerase buffer, which may improve GC rich regime amplification. Such Taq Polymerase buffer may be applied in single cell analysis and next generation sequencing. The buffer components can be adjusted to increase amplification efficiency. For example, the buffer components comprise MgCl₂, KCl, Tris-HCl, Tween 20, and BSA. The buffer components may comprise (alkali earth)(halogen)₂, (alkali)(halogen), Tris-HCl, Tween 20, and BSA. The monovalent halogen anion can be fluorine (F), chlorine (Cl), bromine (Br), iodine (I), astatine (At) or any combination thereof. The alkali monovalent cation can be lithium (Li), sodium (Na), potassium (K), rubidium (Rb), caesium (Cs), francium (Fr), or any combination thereof. The alkali earth bivalent cation may be beryllium (Be), magnesium (Mg), calcium (Ca), strontium (Sr), barium (Ba), radium (Ra), or any combination thereof. One or more of the components can be adjusted to increase amplification efficiency. The concentrations of MgCl₂, KCl, Tris-HCl, Tween 20, and BSA or HSA can be increased or decreased. The concentrations of MgCl₂ and BSA or HSA can be increased, or decreased, to increase amplification efficiency. The concentrations of (alkali earth)(halogen)₂, (alkali)(halogen), Tris-HCl, Tween 20, and BSA or HSA can be increased or decreased. The concentrations of (alkali earth)(halogen)₂ and BSA can be increased, or decreased, to increase amplification efficiency.

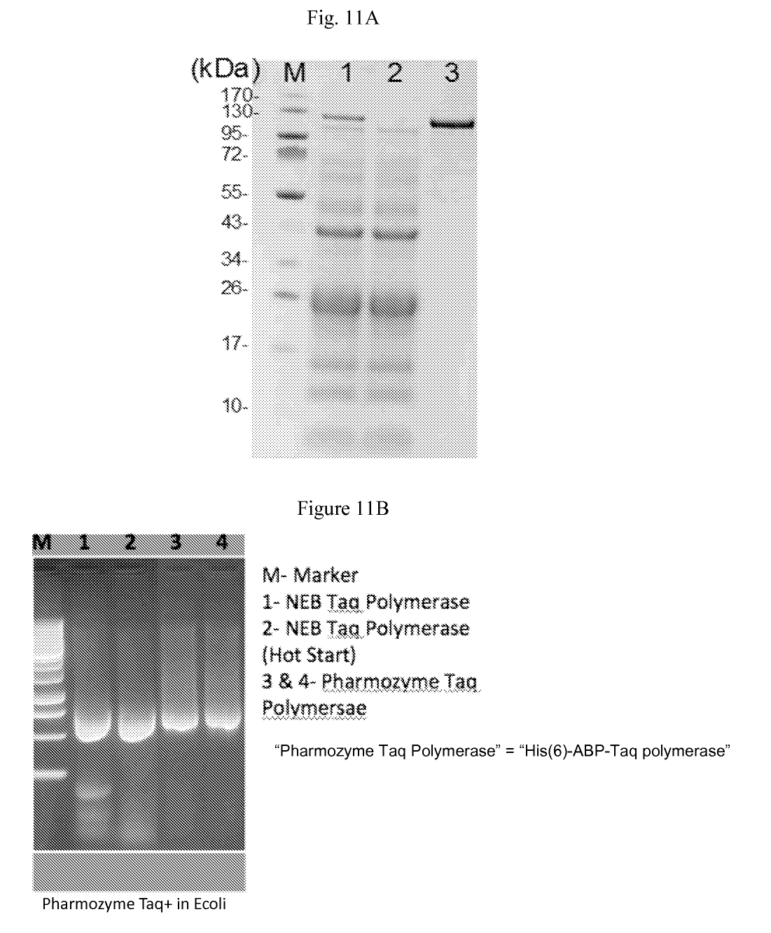

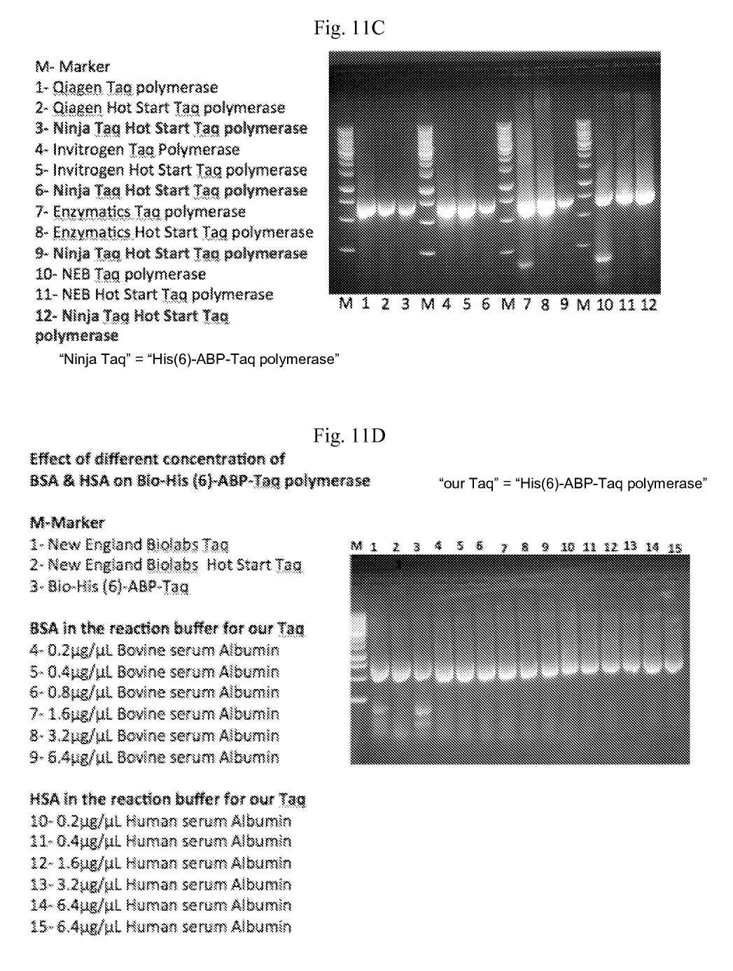

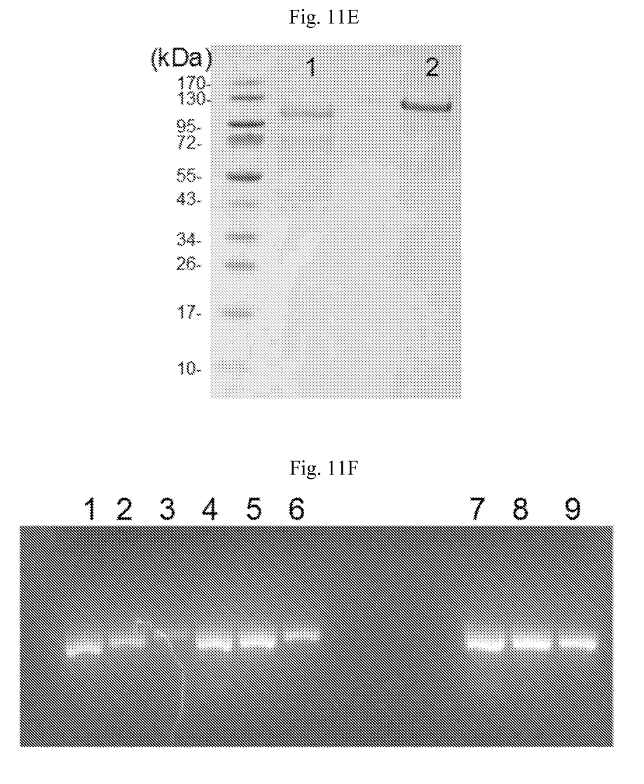

The invention may comprise a detection assay for polynucleotide amplification methodologies. Sometimes, the amplification methodology comprises Real Time PCR. Occasionally, the amplified polynucleotide will have an increased sensitivity relative to commercially available polynucleotide amplification kids (e.g. see FIG. 11C). Such increased sensitivity may be an increase of at least 2, 3, 4, 5, 6, 7, 8, 9, 10, 15, 20, 25, 30, 35, 40, 45, 50, 55, 60, 65, 70, 75, 80, 85, 90, 95, 100, 200, 500, or more in sensitivity.

At times, the invention comprises high sensitivity real time genetic sequencing machine, this sequencing ability may comprise integrated hardware and chemistry technology for high sensitivity genetic amplification analysis (such as Real Time PCR). In one example, the sequencing methodology may include optical sensing of colors (i.e. chromophores), fluorescence or phosphorescence. Sometimes, the sequencing methodology may include sensing of bioluminescence.

In some examples, the invention comprises apparatus, systems, methods and kits for the detection of polynucleotides. As described elsewhere herein, the kit may comprise reagents and buffers for polynucleotide (e.g. DNA) amplification, additional enzymes to further facilitate the amplification process, or to allow quantification of the amplification process.

The amplification methodology may comprise a polymerase construct incorporating a protein sequence able to bind albumin which may or may not require anti-polymerase antibody such as anti-Taq polymerase antibody as is used in the art. The albumin can be serum albumin. The albumin may be mammalian albumin (e.g. human or bovine albumin). Anti-Taq polymerase antibody may keep the Taq DNA polymerase from being activated at storage conditions, (e.g. lower temperature) prevents nonspecific amplification and primer-dimer formation during PCR amplification. Anti-Taq polymerase may include anti-Taq polymerase monoclonal antibodies from eENZYME LLC, BIORON, GeneON, or TOYOBO, and AccuStart™ Taq antibody (Quanta BioSciences).

The polymerase (e.g. Taq polymerase) construct incorporating a protein sequence able to bind albumin may be used with any suitable polynucleotide sequencing techniques. Such sequencing techniques may comprise conventional sequencing methodologies such as Sanger sequencing, Illumina (Solexa) sequencing, pyrosequencing, next generation sequencing, Maxam-Gilbert sequencing, chain termination methods, shotgun sequencing, bridge PCR. Next generation sequencing methodologies may comprise Massively parallel signature sequencing, Polony sequencing, SOLiD sequencing, Ion Torrent semiconductor sequencing, DNA nanoball sequencing, Heliscope single molecule sequencing, Single molecule real time (SMRT) sequencing. Other sequencing methodologies that may be used comprise Nanopore DNA sequencing, Tunnelling currents DNA sequencing, Sequencing by hybridization, Sequencing with mass spectrometry, Microfluidic Sanger sequencing, Microscopy-based techniques, RNA Polymerase sequencing, In vitro virus high-throughput sequencing, or any other sequencing methodologies used in the art.

The methodology may further comprise the use of DNA ligase such as T4 ligase, E-coli ligase, any mammalian ligase such as DNA ligase I, DNA ligase III, DNA ligase IV, eukaryotic DNA ligase, thermostable ligase, or any other ligase known in the art.

The amplification methodologies can be used to amplify the polynucleotides. Polynucleotide amplification may include any amplification such as polymerase chain reaction (PCR), nucleic acid sequence based amplification (NASBA), self-sustained sequence replication (3SR), loop mediated isothermal amplification (LAMP), strand displacement amplification (SDA), whole genome amplification, multiple displacement amplification, strand displacement amplification, helicase dependent amplification, nicking enzyme amplification reaction, recombinant polymerase amplification, reverse transcription PCR, ligation mediated PCR, methylation specific PCR or any other amplification known in the art. Whole Genome Amplification Applications may include IVF, CTC Cancer Detection, single cell research, Stem Cell Research require sample preservation or clean amplification. In some examples of the invention, amplification methodologies used herein include amplification reactions that release pyrophosphate during the amplification process of a polynucleotide strand.

### Taq Polymerase Construct

In some cases, disclosed herein is a polynucleotide amplification enzyme that comprises an amplification enzyme and a protein able to bind to albumin. The amplification enzyme may be a polymerase protein. The polymerase protein may be a DNA polymerase. Exemplary DNA polymerases are disclosed elsewhere herein, and may include Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Taq® DNA polymerase, One Taq® Hot Start DNA polymerase, phi29 DNA polymerase, Phusion® Hot Start Flex DNA polymerase, Phusion® High-Fidelity DNA polymerase, Q5®+Q5® Hot Start DNA polymerase, Sulfolobus DNA polymerase IV, T4 DNA polymerase, Theminator™ DNA polymerase, Vent_{R}™ DNA polymerase, and Vent_{R}™ (exo-) DNA polymerase. In some cases, the polymerase protein is a Taq polymerase. In some cases, the Taq polymerase is a native Taq polymerase or a modified Taq polymerase. In some cases, the Taq polymerase is a modified Taq polymerase. In some cases, the protein able to bind to albumin contains an albumin binding site (ABS).

The modified Taq polymerase construct may contain the modified Taq polymerase portion and an ABS portion. The modified Taq polymerase construct may further comprise a polyhistidine-tag (6×His-tag). The modified Taq polymerase construct may further comprise a polyhistidine-tag (6×His-tag) and ABS. In some instances, HIS(6) (i.e. 6×His-tag) is used as a purification tag. Sometimes, ABP is used as a purification tag. See FIG. 3 as an example. In some cases, ABS is incorporated in an immunoglobulin-binding protein. ABS may be incorporated in Protein G such as in Streptococcal Protein G. ABS may incorporate albumin binding protein (ABP). At times, ABS is ABP, such as ABP from Streptococcal Protein G. In some instances, a fusion Taq polymerase protein is referred to as His(6)-ABS-Taq polymerase. The His(6) tag, ABS, and Taq polymerase may be directly connected to each other. The His(6) tag, ABS, and Taq polymerase may be connected through spacers. Exemplary His(6)-ABS-Taq polymerase constructs are shown in FIG. 3. In some cases, the His(6)-ABS-Taq polymerase construct is the construct shown as 901, 902, 903, 904, 905, or 906. The His(6)-ABS-Taq polymerase construct may be the construct shown as 901. The His(6)-ABS-Taq polymerase construct may further be modified to remove the 6×His-tag portion, shown as 907. The 6×His-tag portion may be cleaved off during or post purification process. Spacer E may contain an enzyme cleavage site, which allows removal of the 6×His-tag from the ABS-Taq polymerase portion. The His(6)-ABS-Taq polymerase construct may be further modified to remove both the 6×His-tag portion and the ABS portion, shown as 908. Spacer F may also contain an enzyme cleavage site. At times, the enzyme cleavage sites in spacer E and spacer F are the same. The enzyme cleavage sites in spacer E and spacer F may be different.

The distance between either the ABS, the HIS(6) or both, and the modified Taq polymerase protein is defined by spacer E and spacer F. Both spacer E and spacer F represent molecule linkage such as covalent linkage. Spacer E may be at least 1, 2, 3, 4, 5, 6, 7, 8, 9, 10, or more covalently linked amino acids. Spacer F may be at least 1, 2, 3, 4, 5, 6, 7, 8, 9, 10, or more covalently linked amino acids. The number of covalently linked amino acids in spacer E may be different than the number of covalently linked amino acids in spacer F. The number of covalently linked amino acids in spacer E may be the same as the number of covalently linked amino acids in spacer F. The spacer molecule may be a chimeric peptide, an organic molecule, saccharide, a peptide, a polynucleotide or a nucleic acid monomer. The organic molecule may be aliphatic, conjugated or aromatic. The conjugated organic molecule may comprise at least 1, 2, 3, 4, 5, 6, 7, 8, 9, 10 or more conjugated bonds. The saccharide may be a mono, di, oligo or poly saccharide. In some examples spacer E is identical to spacer F. In some instances, spacer E is different than spacer F.

As disclosed above, in some instances ABS is obtained from Protein G. Protein G is an immunoglobulin-binding protein that serves as a bacterial receptor on the surface of Gram-positive bacteria. In some instances, Protein G is expressed in group C and group G of Streptococcal bacteria. ABP may be obtained from Streptococcal protein G (SpG), strain G148.

Any portions of the Protein G protein containing ABS may be connected to the modified Taq polymerase. In some cases, the ABS is ABP. Sometimes, ABS is at least one of ABD1, ABD2, and ABD3 regions of the Protein G. BB region may be connected to Taq polymerase. ABP region may be connected to Taq polymerase. ABD region may be connected to Taq polymerase. In some cases, the entire Streptococcal protein G is connected to Taq polymerase. In some instances, the His(6) moiety is further connected to the modified Taq polymerase construct with ABS. The His(6) moiety may be connected to Taq polymerase (such construct lacks an albumin binding site).

In some instances, the ABS further comprises a second binding site. The second binding site may be within the ABP. The second binding site may be linked to the ABP, either through direct covalent linkage or non-directly (e.g. a spacer). The second binding site may be at a site different from the ABP binding site and does not interfere with the interaction of ABP with albumin. The second binding site may be a binding site that recognizes a domain of a membrane protein. The membrane protein can be a type I membrane protein from a bacterium. The membrane protein can be a Staphylococcal protein A (SpA). The second binding site can recognize one or more domains of SpA. Sometimes, the second binding site recognizes domain B of SpA. Occasionally, the second binding site recognizes an analog of domain B, Z domain. In some cases, the second binding site is Z domain binding site. In some instances, the ABP further comprises the Z domain binding site, referred herein as ABP-Z. The ABS construct may be produced using the general procedure for production of the target protein construct. In some cases, the ABS construct is described in FIG. 2.

The modified Taq polymerase can be generated through any suitable mutagenesis methods. In some instances, the modified Taq polymerase is generated through a site-directed mutagenesis method. As disclosed above, site-directed mutagenesis is a method that allows specific alterations or modifications within the gene of interest. The site-directed mutagenesis can utilize Cassette mutagenesis method, PCR-site-directed mutagenesis, whole plasmid mutagenesis, Kunkel's method, or in vivo site-directed mutagenesis method. The modified Taq polymerase can be generated through random mutagenesis method. Random mutagenesis is a method of generating a library of protein mutants with different functional properties. Random mutagenesis can be achieved using error-prone PCR approach, rolling circle error-prone PCR approach, mutator strains approach, temporary mutator strains approach, insertion mutagenesis approach, ethyl methanesulfonate approach, the nitrous acid approach, or DNA shuffling. In some instances, random mutagenesis utilizing an error-prone PCR approach is used to generate a modified Taq polymerase.

In some instances, amplification enzyme and the albumin construct is thermostable. The amplification enzyme may be a polymerase protein. The polymerase protein may be a DNA polymerase. Exemplary DNA polymerases are described elsewhere herein, and may include Bst DNA polymerase, Bsu DNA polymerase, Crimson Taq DAN polymerase, Deep Vent_{R}™ DNA polymerase, Deep Vent_{R}™ (exo-) DNA polymerase, E. coli DNA polymerase I, Klenow fragment (3′-5′ exo-), DNA polymerase I (large Klenow fragment), LongAmp® Taq DNA polymerase, LongAmp® Hot Start Taq DNA polymerase, M-MuLV reverse transcriptase; One Taq® DNA polymerase, One Taq® Hot Start DNA polymerase, phi29 DNA polymerase, Phusion® Hot Start Flex DNA polymerase, Phusion® High-Fidelity DNA polymerase, Q5®+Q5® Hot Start DNA polymerase, Sulfolobus DNA polymerase IV, T4 DNA polymerase, Theminator™ DNA polymerase, Vent_{R}™ DNA polymerase, and Vent_{R}™ (exo-) DNA polymerase. The DNA polymerase may be a Taq polymerase. The amplification enzyme and the albumin construct may be the Taq polymerase and albumin construct. The Taq polymerase and albumin construct may be thermostable. The thermo stable Taq-polymerase and albumin construct may be stable in temperature of at least 50, 51, 52, 53, 54, 55 or more degrees Celsius. In some cases, disclosed herein is a modified hexokinase that has affinity for ATP and a similar affinity for at least two other dNTPs. The dNTPs can be natural nucleosides or non-natural nucleosides. dNTPs may include dATP, dGTP, dTTP, dCTP or dUTP. The binding affinity of each of the nucleotides to the hexokinase may be of at least 1, 5, 10, 20, 30, 40, 50, 60, 70, 80, 90, 100, 110, 120, 130, 140, 150, 160, 170, 180, 190, 200, 210, 220, 230, 250 micromolar (μM) or more. The binding affinity of the nucleotides to the hexokinase may be of at most 1, 5, 10, 20, 30, 40, 50, 60, 70, 80, 90, 100, 110, 120, 130, 140, 150, 160, 170, 180, 190, 200, 210, 220, 230, 250 nanomolar (nM) or less. Thus a binding affinity of the various nucleotides that is similar may be in the range defined by the above-mentioned minimum and maximum values. For example, the similar binding affinity can be between 50 and 150 micromolar (μM). The similar binding affinity can be between 70 and 100 micromolar (μM). In some cases, disclosed herein is a modified luciferase that recognizes ATP but does not recognize dATP. In some cases, disclosed herein are thermostable enzymes that allow the sequencing reaction to take place at a higher temperature. In some instances, the higher temperature in temperature of at least 50, 51, 52, 53, 54, 55 or more degrees Celsius. Sometimes, the higher temperature eliminates the requirement for accessory proteins, such as single-strand DNA binding protein (SSB), to prevent DNA secondary structure formation. In some cases, the sequencing method described herein is as shown in FIG. 4.

In FIG. 4, dNTPs are incorporated in an iterative manner onto the DNAₜₑₘₚₗₐₜₑ by a polynucleotide (e.g. DNA) polymerase such as Taq polymerase. The polymerase may be an enzyme construct that incorporates a protein able to bind albumin, for example a Taq polymerase construct with ABS. The polymerase may be a Taq polymerase construct capable of binding to albumin. In some instances the albumin is human or bovine albumin. Albumin may be serum albumin (e.g. human serum albumin or bovine serum albumin). The albumin may be any albumin type, for example bovine albumin or human albumin. The albumin may be serum albumin. Sometimes, the introduction of each type of dNTP is controlled. The dNTP type may be introduced one by one into the reaction mixture. In some instances, both a DNA_{product} and an inorganic pyrophosphate (PPi) are generated (201). In a coupled reaction, the PPi in the presence of APS is converted into ATP by ATP sulfurylase (202). ATP is subsequently detected by luciferase in a light generating reaction (203). Excess nucleotides such as ATP and dNTPs are converted by a saccharide phosphorylating enzyme (e.g. hexokinase) in the presence of a saccharide (e.g. hexose) into ADP and dNDPs, while the hexose is converted into saccharide (e.g. hexose) phosphates (204). In some cases, the amount of light produced is proportional to the amount of nucleotides incorporated. In some instances, the overall output forms a light profile which can be qualitatively or quantitatively monitored.

In some instances, the Taq polymerase construct is produced using the general procedure for production of the target protein construct. Sometimes, the Taq polymerase construct is produced using by a bioengineered host as mentioned above. As described elsewhere herein, purification of the modified Taq polymerase protein can utilize the general procedure for purification of the target protein construct described above, where the target protein is the modified the modified Taq polymerase protein. In some cases, the purification scheme is the scheme illustrated in FIG. 2.

### Applications in the Field of Molecular Diagnostics

In some aspects of the invention, the invention is useful in molecular diagnostic fields such as infectious agent identification, hereditary diseases, cancer genetic testing, and genetic variations such as single-nucleotide polymorphism. The invention disclosed herein may be useful for identification of infectious agents. As used herein, an infectious agent is an agent such as a virus, a bacterium, a fungus, a nematode or a protozoan that causes an infection in a host organism such as a mammal, such as a human.

Exemplary infectious virus include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arena viridae (hemorrhagic fever viruses); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses′); Poxyiridae (variola viruses, vaccinia viruses, pox viruses), Iridoviridae (e.g., African swine fever virus), or unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Exemplary infectious bacteria include Helicobacter pyloris, Borelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, or Actinomyces israelli.

Exemplary infectious fungi include: Cryptococcus neoformans, Histoplasma capsulatum, Coccidioides immitis, Blastomyces dermatitidis, Chlamydia trachomatis or Candida albicans.

Exemplary infectious nematode include: ascarids (Ascaris), filarias, hookworms, pinworms, roundworms or whipworms.

Exemplary infectious protozoan include Acanthamoeba, Balamuthia mandrillaris, Endolimax, Entamoeba histolytica, Giardia lamblia or Plasmodium spp.

In some instances, the invention disclosed herein is useful for identification of hereditary conditions. Exemplary hereditary conditions include 22q11.2 deletion syndrome, Angelman syndrome, canavan disease, Charcot-Marie-Tooth disease, color blindness, Cri du chat, cystic fibrosis, Down syndrome, Duchenne muscular dystrophy, Haemochromatosis, haemophilia, Klinefelter syndrome, neurofibromatosis, phenylketonuria, polycystic kidney disease, Prader-Willi syndrome, sickle-cell disease, Tay-Sachs disease, or Turner syndrome.

In some instances, the invention disclosed herein is useful for cancer genetic testing, for example, for optimizing a therapeutic regiments or for stratifying a patient population into drug-resistant or drug-naïve patients, based on the type of cancer present. In some cases, the cancer is a solid tumor or a hematologic cancer. In some cases, the solid tumor is a sarcoma or a carcinoma. In some cases, the hematologic cancer is a leukemia, a lymphoma, a myeloma, a non-Hodgkin's lymphoma, a Hodgkin's lymphoma, or a B-cell malignancy.

Exemplary sarcoma include: alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epithelioid cell differentiation; osteosarcoma; parosteal osteosarcoma; neoplasm with perivascular epithelioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma or telangiectatic osteosarcoma.

Exemplary carcinoma include: anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer.

Exemplary T-cell malignancy include: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

Exemplary B-cell malignancy include: chronic lymphocytic leukemia (CLL), small lymphocytic lymphoma (SLL), high risk CLL, or a non-CLL/SLL lymphoma. In some instances, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, the invention disclosed herein is useful for detecting the presence of genetic variations in an individual. As used herein, genetic variations include deletion and insertion of one or more nucleotides, translocations of different nucleotide occurrences (e.g. single point mutations such as SNPs or a base-pair substitution), or variations in the number of multiple nucleotide repetitions. In some cases, the genetic variation is a single-nucleotide polymorphism (SNP). SNPs are single nucleotide variations and can sometimes lead to diseases. In some cases, a SNP is a common SNP or a rare SNP, due to the rate of occurrences in a population. In some cases, the invention disclosed herein is useful for detecting the presence of a common SNP. In some cases, the invention disclosed herein is useful for detecting the presence of a rare SNP.

### Instrumentations

In some aspects of the invention, the invention comprises a genetic detection machine, device, apparatus or system. Such machine, device, apparatus or system may comprise one or more of the following: compartments for the abovementioned specialized reagents, sample preparation compartment, reservoirs, mechanism for the detection of bioluminescence (e.g. optical detector), a heating element, a cooling element, a sample moving element (i.e. pushing or suction devise such as one or more pumps), a sample mixing element (i.e. stirrer, mixer, vortex), channels (e.g. closed channels, open channels, microfluidic channels), a specialized computer incorporating bioinformatics software, software and an output device (e.g. sound, display, vibration or printer). In some examples, the software allows controls of the operation of programming and running methods of one or the more compartments or instruments, monitoring the status or processing of the results. Sometimes, the software further allows communication with one or more additional machines, devices, apparatus or systems or a centralized command system, or any combination thereof. Sometimes, the reagents comprise chemically modified enzymes (e.g. mutated luciferase that does not recognize dATP, coupled with HIS(6) and/or ABS, luciferase construct with HIS(6) and/or ABS, hexokinase construct with HIS(6) and/or ABS, ATP sulfurylase), specialized polynucleotide amplification enzyme (e.g. polymerase construct incorporating a protein sequence able to bind albumin), nucleosides, target polynucleotide to be amplified and sequenced, polynucleotide primers. The specialized amplification enzyme may be able to copy any length of polynucleotide fragments such as short, medium, long polynucleotide fragments or whole genome. Polynucleotides may be DNA or RNA. Sometimes, the methods are modified that the amplified DNA becomes immobilized or is provided with means for attachment to a solid support. For example, a PCR primer may be immobilized or be provided with means for attachment to a solid support. Also, vectors may comprise means for attachment to a solid support. Sometimes, the machine, device, apparatus or system may comprise a microfluidic device.

In some aspects of the invention, disclosed is a system for polynucleotide sequencing comprising amplifying at least one polynucleotide to be amplified by hybridizing nucleoside-polyphosphate molecule to at least one polynucleotide to be amplified in a complementary fashion, linking the hybridized nucleoside-polyphosphate molecule to form polynucleotide strand complementary to at least one polynucleotide to be amplified, and releasing at least one pyrophosphate. The linkage may be covalent linkage. Also disclosed in the system are reagents and enzymes involved in converting at least one pyrophosphate to ATP and reagents and enzymes disclosed herein involved in phosphorylating at least one saccharide by reacting the ATP in the presence of an enzyme that phosphorylates at least one saccharide to produce at least one phosphorylated saccharide. The polyphosphate may incorporate at least 3, 4, 5, 6, 7, 8 or more phosphates. The system may contain integrated modules in which each module is tasked with, for example, sample preparation, amplification, or reaction monitoring. One or more of the modules may contain specialized software which allow for completion of the tasks for the one or more modules. In some cases, one of the modules is a microfluidic device. The system may contain separate units, which functions either individually to complete a portion of the processes disclosed in the invention, or functions in tandem to complete the processes disclosed in the invention. Each individual unit may be tasked with, for example, sample preparation such as a preparation kit, amplification such as a PCR machine, or reaction monitoring such as with a luminometer. One or more of the individual units may contain different software. In some cases, one of the units is a microfluidic device.

The present disclosure provides computer control systems that are programmed to implement methods of the disclosure. FIG. 8 shows a computer system 1001 that is programmed or otherwise configured to control the genetic detection system. The computer system 1001 can regulate various aspects of the flow of the single fluid phase within the genetic detection system of the present disclosure, such as, for example, control various components of the genetic detection system to detect polynucleotide sequence such as single stranded or double stranded polynucleotides (such as RNA, DNA or any modified or non-natural polynucleotide sequence). The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. Examples of operations performed by the CPU 1005 can include fetch, decode, execute, and write back.

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., operator or end user). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030. In some cases, the end user is a lab technician, a physician, a customer, a patient, or a researcher.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display that comprises a user interface (UI) for providing, for example, various aspects of the genetic systems. An example for an aspect can be the level of a polynucleotide detected by the system. The display may optionally include the absolute or relative polynucleotide levels, the sequence of the polynucleotides and data regarding any genetic alterations as well as historical genetic data of the user or any comparative or normal polynucleotide data. Any of the abovementioned polynucleotide levels, as well as the corresponding historical or comparative date and time in which the levels were collected, can be saved in any of the abovementioned storage systems or their combination, and can be accessed by the computer system and/or by a user. Saving may be effectuated by the computer system, by a user, or by both. The historical data may be accessible by the computer system, by a user, or by both. The historical levels displayed may be of a past date and/or time chosen by the user, or of a predetermined date and/or time. The display may also comprise a sketch of all the components of the genetic detection system. The sketch may display the current operational status of the particular component. The sketch may display the level of reactants, polynucleotides, solvents, buffers, enzymes, any other property of the fluid within the system, or any combination thereof. The sketch may also display the abovementioned real time levels, historical levels, or both. The display may further include a user interface that may allow manual control of any of the components of the hypersensitive genetic detection system. Such user control may be effectuated by the user using a touch screen, a remote control device, computer “mouse,” keypad, keyboard, touchpad, stylus, joystick, thumb wheel, voice recognition interface, any other user input interface known in the art, or a combination thereof. Examples of UI's include, without limitation, a graphical user interface (GUI) and web-based user interface.

Methods and systems of the present disclosure can be implemented by way of one or more algorithms. An algorithm can be implemented by way of software upon execution by one or more computer processors. In some examples, one or more algorithms for comparing or evaluating sequencing data may be used.

### Compositions and Kits

This disclosure also provides compositions and kits for use with the methods described herein. The compositions may comprise any component, reaction mixture and/or intermediate described herein, as well as any combination thereof. For example, the disclosure provides detection reagents for use with the methods provided herein. Any suitable reagent may be provided, including HIS(6), Z domain, and/or ABS polymerase (e.g. DNA polymerase), HIS(6) and/or ABS hexokinase, mutated luciferase that does not recognize dATP, HIS(6), Z domain, and/or ABS luciferase, HIS(6) and/or ABS mutated luciferase that does not recognize dATP, chemically modified hexokinase, chemically modified HIS(6) and/or ABS hexokinase, chemically modified mutated luciferase that does not recognize dATP, chemically modified HIS(6) and/or ABS luciferase, chemically modified HIS(6) and/or ABS mutated luciferase that does not recognize dATP, chemically modified luciferase, ABS sulfurylase, chemically modified ABS sulfurylase, apyrase, chemically modified apyrase, or any combination thereof. Additional detection reagents may include primers for hybridization to target DNA, deoxynucleotides, or optionally deoxynucleotide analogues, optionally including, in place of dATP, a dATP analogue which is capable of acting as a substrate for a polymerase but incapable of acting as a substrate for a PPi-detection enzyme, and buffers for carrying out the PCR reaction. In some instances, the primers are sufficiently complementary to the target DNA to allow annealing.

In some instances, this disclosure provides a kit for polynucleotide sequencing comprising: polynucleotide amplification reagent; saccharide phosphorylating enzyme; and bioluminescent enzyme, wherein the kit excludes a nucleotide degrading enzyme. The saccharide phosphorylating enzyme may be any of the abovementioned saccharide phosphorylating enzymes, and the bioluminescent enzyme may be any of the abovementioned bioluminescent enzymes. The kit may further comprise an enzyme converting pyrophosphate to ATP. An enzyme converting pyrophosphate to ATP may be ATP sulfurylase. Sometimes the kit further comprises DNA ligase. An example for a DNA ligase is T4 ligase. The kit may further comprise a processing unit. An example for a processing unit may be a computer. The kit may further comprise specialized software, a light detection system, a detection system for the detection of electro-magnetic radiation, an input unit, or an output unit. An example for an output unit may be audio device, a visual devise or a printer. A visual devise may be a screen such as a computer screen. The kit may further comprise channels. In some cases, the channels are microfluidic channels. In some cases, the bioluminescent enzyme does not recognize dATP. The bioluminescent enzyme may be thermostable above 50 degrees Celsius. The kit may further comprise a saccharide phosphorylating enzyme. The kit may further comprise a saccharide.

In some instances, this disclosure provides a kit for polynucleotide sequencing comprising: polynucleotide amplification reagent; and bioluminescent enzyme that does not recognize dATP, wherein the kit excludes a bioluminescent enzyme that recognizes dATP. The saccharide phosphorylating enzyme may be any of the abovementioned saccharide phosphorylating enzymes, and the bioluminescent enzyme may be any of the abovementioned bioluminescent enzymes. The kit may further comprise an enzyme converting pyrophosphate to ATP. An enzyme converting pyrophosphate to ATP may be ATP sulfurylase. Sometimes the kit further comprises DNA ligase. An example for a DNA ligase is T4 ligase. The kit may further comprise a processing unit. An example for a processing unit may be a computer. The kit may further comprise specialized software, a light detection system, a detection system for the detection of electro-magnetic radiation, an input unit, or an output unit. An example for an output unit may be audio device, a visual devise or a printer. A visual devise may be a screen such as a computer screen. The kit may further comprise channels. In some cases, the channels are microfluidic channels. In some cases, the bioluminescent enzyme is thermostable above 50 degrees Celsius. The kit may further comprise a saccharide phosphorylating enzyme. The kit may further comprise a saccharide. The kit may exclude a nucleotide degrading enzyme. In some cases, the kit further comprises a nucleotide degrading enzyme. An example for a nucleotide degrading enzyme is apyrase.

In some instances, this disclosure provides a kit for polynucleotide sequencing comprising: a polynucleotide amplification reagent; and a bioluminescent enzyme that is stable above 50 degrees Celsius, wherein the kit excludes a bioluminescent enzyme that is stable up to 50 degrees Celsius. The saccharide phosphorylating enzyme may be any of the abovementioned saccharide phosphorylating enzymes, and the bioluminescent enzyme may be any of the abovementioned bioluminescent enzymes. In some cases, the bioluminescent enzyme does not recognize dATP. The kit may further comprise an enzyme converting pyrophosphate to ATP. An enzyme converting pyrophosphate to ATP may be ATP sulfurylase. Sometimes the kit further comprises DNA ligase. An example for a DNA ligase is T4 ligase. The kit may further comprise a processing unit. An example for a processing unit may be a computer. The kit may further comprise specialized software, a light detection system, a detection system for the detection of electro-magnetic radiation, an input unit, or an output unit. An example for an output unit may be audio device, a visual devise or a printer. A visual devise may be a screen such as a computer screen. The kit may further comprise channels. In some cases, the channels are microfluidic channels. The kit may further comprise a saccharide phosphorylating enzyme. The kit may further comprise a saccharide. In some examples, the kit excludes a nucleotide degrading enzyme. In some cases, the kit further comprises a nucleotide degrading enzyme. An example for a nucleotide degrading enzyme is apyrase.

In some instances, this disclosure provides a kit for polynucleotide sequencing comprising: polynucleotide amplification reagent; a thermostable enzyme which inactivates at least one nucleoside-polyphosphate; and a bioluminescent enzyme. The enzyme which inactivates at least one nucleoside-polyphosphate may be any of the abovementioned saccharide phosphorylating enzymes, or a nucleoside degrading enzyme. An example for a saccharide phosphorylating enzyme is hexokinase. An example for a nucleoside degrading enzyme is apyrase. The bioluminescent enzyme may be any of the abovementioned bioluminescent enzymes. In some cases, the bioluminescent enzyme does not recognize dATP. The kit may further comprise an enzyme converting pyrophosphate to ATP. An enzyme converting pyrophosphate to ATP may be ATP sulfurylase. Sometimes the kit further comprises DNA ligase. An example for a DNA ligase is T4 ligase. The kit may further comprise a processing unit. An example for a processing unit may be a computer. The kit may further comprise specialized software, a light detection system, a detection system for the detection of electro-magnetic radiation, an input unit, or an output unit. An example for an output unit may be audio device, a visual devise or a printer. A visual devise may be a screen such as a computer screen. The kit may further comprise channels. In some cases, the channels are microfluidic channels. The kit may further comprise a saccharide phosphorylating enzyme. The kit may further comprise a saccharide. In some examples, the kit excludes a nucleotide degrading enzyme. In some examples, the kit excludes a saccharide phosphorylating enzyme.

The present disclosure also provides kits for carrying out the methods of the invention. Accordingly, a variety of kits are provided in suitable packaging. The kits may be used for any one or more of the uses described herein, and, accordingly, may contain instructions for detecting the presence or absence of a target DNA, and may contain instructions for interpretation of the results. A kit may be a diagnostic kit, for example, a diagnostic kit suitable for the detection of any infectious agents, hereditary genes, cancer genes and biomarkers, hereditary abnormalities, and/or genetic variations recited herein. A kit may contain any of the compositions provided in this disclosure, including those recited above.

While some illustrations of the present invention have been shown and described herein, it will be obvious to those skilled in the art that such illustrations are provided by way of example only. It is not intended that the invention be limited by the specific examples provided within the specification. While the invention has been described with reference to the aforementioned specification, the descriptions and illustrations of the examples herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the examples of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

While some illustrations of the present invention have been shown and described herein, it will be obvious to those skilled in the art that such illustrations are provided by way of example only. Numerous variations, changes, and substitutions will now occur to those skilled in the art without departing from the invention. It should be understood that various alternatives to the illustrations of the invention described herein may be employed in practicing the invention. It is intended that the following claims define the scope of the invention and that methods and structures within the scope of these claims and their equivalents be covered thereby.

### EXAMPLES

These examples are provided for illustrative purposes only and not to limit the scope of the claims provided herein.

### Example 1: Error Prone PCR for Generation of Modified Luciferase and Modified Hexokinase

E. coli DH5α was used for the E. coli cloning experiments. E. coli Rosetta 2 (DE3) (EMD Millipore, Billerica, Mass., USA) was used as host for the expression experiments. P. pastoris strains KM71, SMD1168 or GS115 were used as host for the yeast expression experiments.

Error prone PCR was used for introducing molecular variety into firefly luciferase and Saccharomyces crevice hexokinase isoenzyme B genes. In this method, it was possible to generate random mutations at any position in the target gene by reducing fidelity of Taq DNA polymerase in the presence of Mn² ions and unbalanced ratios of dNTPs. The GeneMorph II Random Mutagenesis Kit from Agilent Technologies was used. This kit contained Mutazyme II DNA polymerase (2.5 U/μL), 10× Mutazyme II reaction buffer, and a dNTP mix (10 mM each dNTP). 3-4 μg of the plasmid was used for cloning and expression of the epPCR library, employing vectors pET21 and pPIC9 for E. coli and Pichia pastoris, respectively.

Cleavage of the vector (˜3 μg) and epPCR product (˜1 μg) were followed to completion using restriction enzymes that facilitated directional, sticky-ended cloning and cut within the expression vectors multi-cloning site.

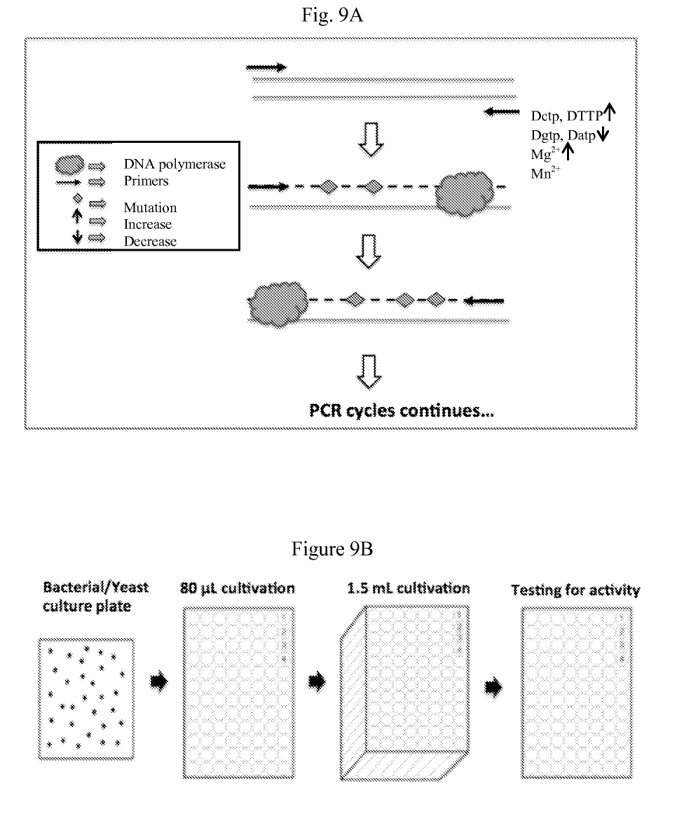

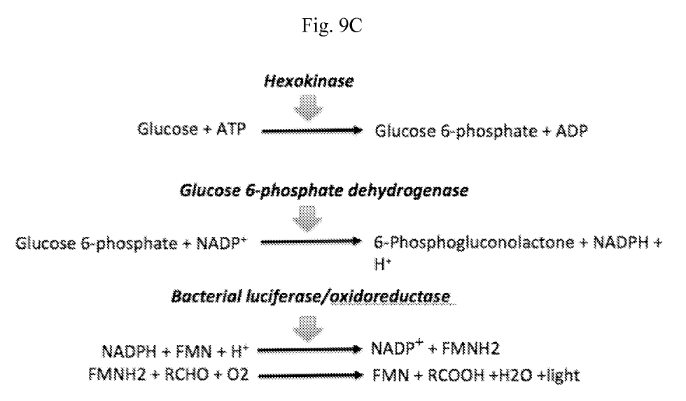

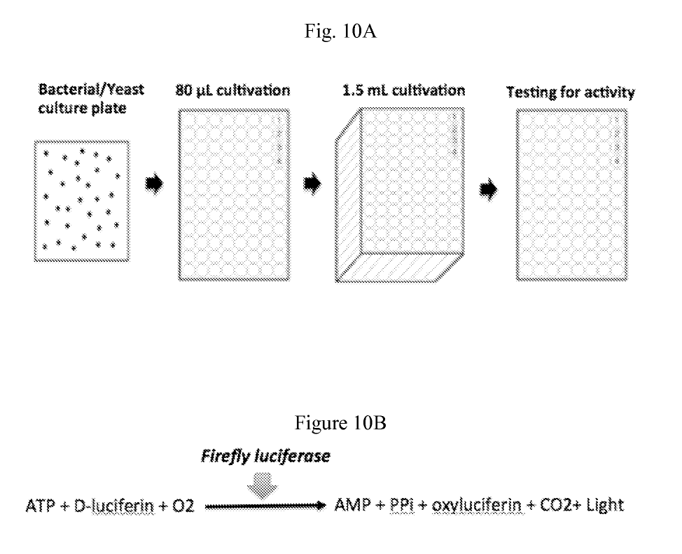

FIG. 9 illustrates the error prone PCR method for generation of modified hexokinase. The activity of the modified hexokinase was further tested with an activity assay using either a bacterial luciferase or an oxidoreductase, as illustrated in FIG. 9C. FIG. 10 illustrates the error prone PCR method for generation of modified firefly luciferase.

### Example 2: Acetylation of Lysine Residues

Acetylation of lysine residues took place using acetic anhydride in 50 mM sodium phosphate at pH 8.0, using 50-fold molar excess of acetic anhydride per mol amino group. A10 ml of a 20 mg/ml protein solution in the phosphate buffer was mixed with one third volume of saturated sodium acetate and the temperature and pH were maintained either at 0-4° C. or at 8.0° C. The reactions were completed in about 1 h with constant stirring.

### Example 3: Citraconylation of Lysine Residues

Citraconylation of lysine residues took place using citroconic anhydride (Khajeh et al., 2001, Enzyme and Microbial Technology, 28, 543-549). The protein concentrations were at 4 mg/ml in 10 ml of 100 mM borate buffer (pH 8.0) and the process was followed at room temperature by step-wise addition of 3 μL aliquots of the modifier while maintaining the pH of the stirred solution at 8.0 by the addition of 2 M NaOH. Upon completion of the reaction, the pH of the solution remained stable, and the reaction mixture was extensively dialyzed against 20 mM Tris, pH 7.5.

### Example 4: Determination of the Extent of Lysine Modification Through Fluorescence

After the completion of acetylation or citraconylation, the extent of the lysine modification in the enzymes was determined. To a 5 μl protein solution at approximately 2 mg/ml, 250 μl 100 mM Na2HPO4, 90 μl deionized water and 100 μl of a 1 mM fluorescamine in acetonitrile were mixed and incubated for 10 min in the dark. Fluorescence was measured using excitation/emission wavelengths of 390/490 nm. (see, Schmitt, et. al, 2005, Anal. Biochem. 338, 201-215, Morshedi et. al, 2010, Biochimica Biophysica Acta, 1804, 714-722).

### Example 5: Error Prone PCR for Generation of Modified Taq Polymerase Cloning

E. coli DH5α was used for the E. coli cloning experiments. E. coli Rosetta 2(DE3) (EMD Millipore, Billerica, Mass., USA) was used as host for the expression experiments. P. pastoris strains KM71, SMD1168 or GS115 were used as host for the yeast expression experiments.

The gene encoding Taq polymerase was amplified from Thermus aquaticus strain YT-1. The gene was cloned into vector pET21, which contains the coding sequence for an N-terminal fusion to the affinity tag Bio-His₆-ABP.

Alternatively, the gene encoding Taq polymerase with an N-terminal fusion to albumin-binding protein (ABP) was synthesized by Genscript (Piscataway, N.J., USA) with the codons optimized for expression in P. pastoris. Flanking XhoI/NotI restriction sites were added such that the coding sequence could be inserted into the vector YpDC541 to create a fusion with the α-mating secretion signal of S. cerevisiae and to be under the control of the methanol-inducible alcohol oxidase promoter. The YpDC541-ABP-Taq construct was integrated into P. pastoris strains KM71, SMD1168 or GS115.

Growth, Expression, and Purification Using the E. coli System

E. coli Rosetta 2 (DE3) cells harboring the plasmid pET21-Tag-Taq were used for expression experiments. Cells were grown at 37° C. in Terrific Broth (47.6 g/l, Sigma-Aldrich, St. Louis, Mo., USA) supplemented with 100 μg/ml of carbenicillin and 34 μg/ml of chloramphenicol until OD₆₀₀ reached 0.6. Isopropyl-β-D-thiogalactoside (IPTG) and D-biotin were added at final concentrations of 0.5 mM and 0.1 mM, respectively. Cells were grown an additional 4 hr at 37° C.

Following recombinant expression of Bio-His₆-ABP-Taq in E. coli, the protein was purified by affinity chromatography. The cells were first centrifuged and resuspended to 1:20 of the original starting volume using wash buffer (50 mM Tris-HCl, pH 8.0, 0.2 M NaCl, 0.05% Tween 20, and 1 mM EDTA). Lysozyme (1 mg/ml), DNAse I (100 U), MgCl₂ (2.5 mM), and CaCl₂ (0.5 mM) were added, and the suspension was incubated at 37° C. for 2 hr. The cells were sonicated, heated to 75° C. for 1 hr, and then centrifuged at 10,000×g for 25 min. After centrifugation, the supernatant was filtrated (0.22 μm) prior to loading onto a 5 ml human serum albumin (HSA)-Sepharose column, which had been made using HSA (Sigma-Aldrich, St. Louis, Mo., USA) and NHS-Sepharose (GE Healthcare, Pittsburgh, Pa., USA). After loading, the column was washed with 150 ml of washing buffer (50 mM Tris-HCl, pH 8.0, 0.2 M NaCl, 0.05% Tween 20, and 1 mM EDTA) followed by a high salt wash buffer (washing buffer with 2 M NaCl) to remove residual DNA contaminants. A pre-elution wash (10 mM NH₄Ac, pH 5.5) of 50 ml was applied next, followed by elution with 10 ml of 0.5 M HAc, pH 2.8. The eluted sample was collected in 1 ml of 1 M Tris-HCl, pH 8.0 and buffer exchanged by ultrafiltration centrifugal concentrators (Vivaproducts, Littleton, Mass., USA) into 2× storage buffer (40 mM Tris-HCl, pH 8.0, 200 mM KCl, 0.2 mM EDTA, 2 mM dithiothreitol). Glycerol, Tween 20, and IGEPAL CA-360 were added to final concentrations of 50%, 0.5%, and 0.5%, respectively, and the sample was stored at −20° C.

### Growth, Expression, and Purification Using the Yeast System

Yeast cultures were grown according to the protocols in the Pichia Expression Kit manual (Invitrogen, Carlsbad, Calif., USA). For biomass accumulation, cultures were grown in YPD medium (1% yeast extract, 2% peptone, 2% glucose) at 30° C. until OD₆₀₀ reached >10. For protein expression, the yeast cells were centrifuged, resuspended at OD₆₀₀ of 10 in BMMY medium (1% yeast extract, 2% peptone, 100 mM potassium phosphate pH 6.0, 1.34% YNB, 4×10⁻⁵% biotin, 1% methanol), and were grown at 30° C. for 2-3 days with an addition of 1% methanol supplementation every 24 hr.

Following recombinant expression of ABP-Taq in P. pastoris, the protein was purified by affinity chromatography. The culture was first centrifuged to remove cells and then passed through a 0.22 μm filter. This solution was then applied to a 5 ml human serum albumin (HSA)-Sepharose column, which had been made using HSA (Sigma-Aldrich, St. Louis, Mo., USA) and NHS-Sepharose (GE Healthcare, Pittsburgh, Pa., USA). After loading, the column was washed with 150 ml of washing buffer (50 mM Tris-HCl, pH 8.0, 0.2 M NaCl, 0.05% Tween 20, and 1 mM EDTA) followed by a high salt wash buffer (washing buffer with 2 M NaCl) to remove residual DNA contaminants. A pre-elution wash (10 mM NH₄Ac, pH 5.5) of 50 ml was then applied, followed by elution with 10 ml of 0.5 M HAc, pH 2.8. The eluted sample was collected in 1 ml of 1 M Tris-HCl, pH 8.0 and buffer exchanged by ultrafiltration centrifugal concentrators (Vivaproducts, Littleton, Mass., USA) into 2× storage buffer (40 mM Tris-HCl, pH 8.0, 200 mM KCl, 0.2 mM EDTA, 2 mM dithiothreitol). Glycerol, Tween 20, and IGEPAL CA-360 were added to final concentrations of 50%, 0.5%, and 0.5%, respectively, and the sample was stored at −20° C.

FIG. 11 illustrates gel electrophoresis of the Taq polymerase-ABS construct following purification from either E. coli or yeast cells. FIG. 11A shows a Coomassie-stained SDS-PAGE gel that shows a Bio-His₆-ABP-Taq polymerase produced in E. coli. Following cell lysis, heating, and centrifugation, the cell lysate was passed over an HSA-Sepharose column for one-step affinity purification. Lane M is the molecular weight ladder (Fisher Scientific, Pittsburgh, Pa., USA). Lane 1 is the cell lysate. Lane 2 is the column flow through. Lane 3 is the column elution. The expected molecular weight of Bio-His₆-ABP-Taq is 113 kDa. FIG. 11B illustrates a gel electrophoresis of amplified DNA. Lines 3 and 4 represent DNA amplified using Taq Polymerase protein construct with ABP and HIS(6), expressed in E. coli, in the absence of anti Taq Polymerase antibodies. FIG. 11C compares the Taq polymerase (Ninja Taq) with several commercially available Taq polymerases. FIG. 11D compares the Taq polymerase (Ninja Taq) with several commercially available Taq polymerases under different concentrations of BSA or HSA. FIG. 11E illustrates a Coomassie-stained SDS-PAGE gel that shows ABP-Taq polymerase produced in P. pastoris. Following centrifugation and filtration, the cell culture supernatant was passed over an HSA-Sepharose column for one-step affinity purification. Lane 1 is column flow-through and lane 2 is column elution. The expected molecular weight of ABP-Taq is 109 kDa. FIG. 11F illustrates an agarose gel showing PCR amplification by Taq polymerase produced in P. pastoris and E. coli. Lanes 1, 2, and 3 illustrate product from batch 1 of Taq polymerase produced in P. pastoris at 1 μL, 0.2 μL, and 0.04 μL of enzyme in 25 μL PCR reactions. Lanes 4, 5, and 6 illustrate product from batch 2 of Taq polymerase produced in P. pastoris at 1 μL, 0.2 μL, and 0.04 μL of enzyme in 25 μL PCR reactions. Lanes 7, 8, and 9 illustrate product from Taq polymerase produced in E. coli at 1 μL, 0.2 μL, and 0.04 μL of enzyme in 25 μL PCR reactions.

### Example 6: Firefly Luciferase Photinus Pyralis Mutants Expression in E. coli Protein Expression

E. coli cells containing pT7Z_{basic} II-Luciferase was grown overnight at 30° C. in a shake flask containing 50 ml of Tryptic Soy Broth (TSB)+Yeast Extract (30 g/l tryptic soy broth, Merck KgaA, Darmstadt, Germany; 5 g/l yeast extract) supplemented by kanamycin (50 mg/l). The following morning, cultivations were inoculated with 10 ml of the overnight culture to 500 ml fresh TSB+YE with kanamycin (50 mg/l) in 5 liters shake flask. The cells were grown at 30° C. until OD_{600 nm} reached approximately 1.0. Then the protein production was induced by addition of isopropyl-β-D-thiogalactopyranoside (IPTG) to a final concentration of 1 mM and the cultivation was continued for three and half-hours.

### Protein Purification Using Zbasic Tag:

Cation exchange. An pre-packed column containing 5 ml S-Sepharose FF cation exchange resin (GE Health, USA) was pre-equilibrated with 5 column volumes (CVs) of 50 mM Tris-HCl, pH 7.5 with 200 mM NaCl (buffer A). The sample was loaded onto the column at a flow rate of 3 ml/min, using the liquid chromatography system ÄKTA FPLC (GE Health, USA). The unbound material was washed out from the column with approximately 5 to 10 CVs of buffer A at a flow rate of 3 ml/min until the absorbance at 280 nm was below 50 mAU. Z_{basic}-Luciferase mutations were eluted using a linear NaCl gradient (0.2-1) M/20 CV. Eluted protein was collected in 5 ml-fractions and screened for luciferase activity. The fractions that showed luciferase activity was analyzed by SDS-PAGE and relevant fractions were pooled.

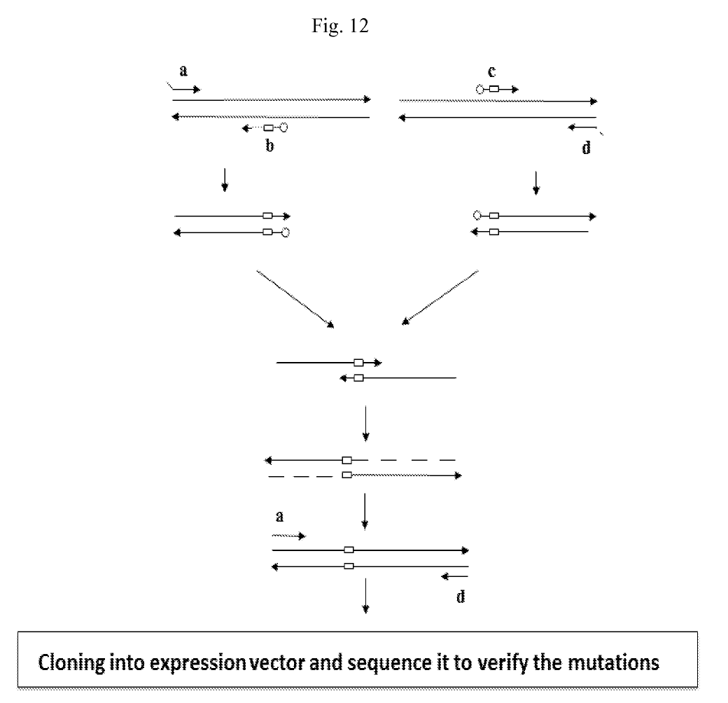

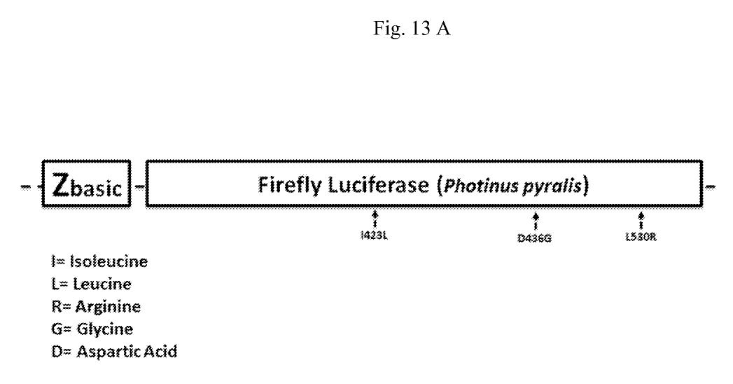

FIG. 12 shows an exemplary PCR mutagenesis of a modified luciferase described herein.

FIG. 13 illustrates the luciferase activity of exemplary modified luciferases described herein. FIG. 13A shows a wild type luciferase with 3 mutations (I423L, D436G and L530R). FIG. 13B shows Thermo wild type luciferase which has five mutations (T214A, I232A, F295L, E354K, and L550V). FIG. 13C shows mutant luciferase with 8 mutations, T214A, I232A, F295L, E354K, L550V, I423L, D436G and L530R. FIG. 13D shows Thermo wild type luciferase which has four mutations (T214A, I232A, F295L, and I423L).

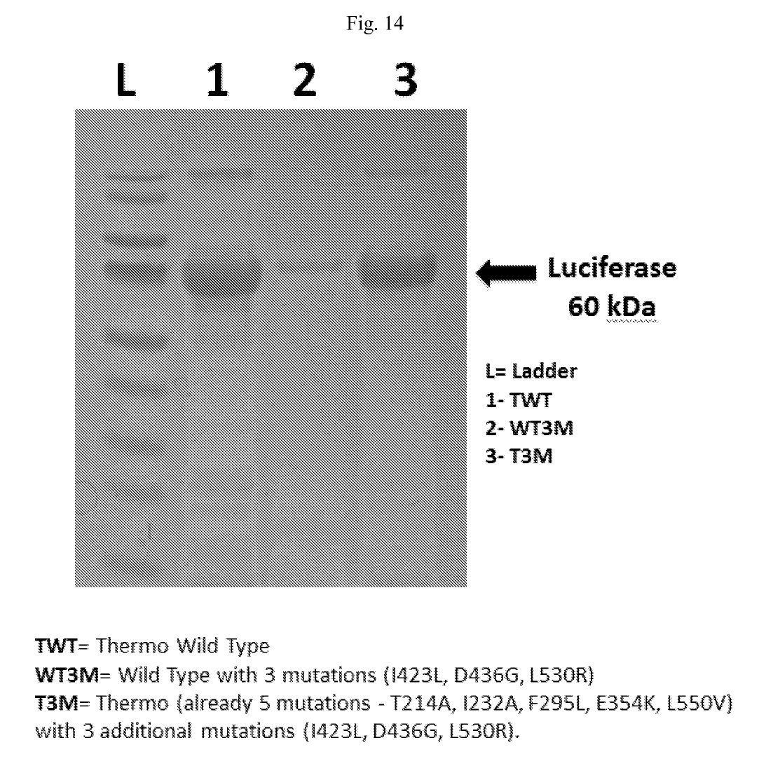

FIG. 14 shows an illustrative SDS-PAGE on modified luciferases with different mutations. TWT refers to Thermo Wild Type with mutations at T214A, I232A, F295L, E354K, and L550V. WT3M refers to a modified luciferase with mutations at I423L, D436G and L530R. T3M refers to a modified luciferase with mutations at T214A, I232A, F295L, E354K, L550V, I423L, D436G and L530R.

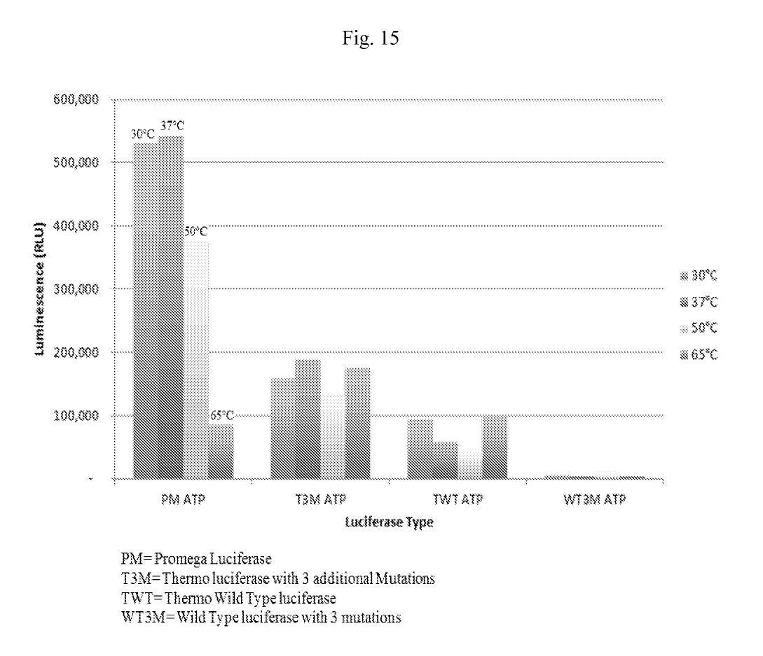

FIG. 15 shows the testing of various modified luciferase described herein at four different temperatures. The reaction was carried out by the addition of 1 ul of the luciferase enzyme in a 1 mM ATP reaction. Promega's luciferase lost activity as the temperature increased but the modified luciferase described herein maintained their activity over the various temperatures illustrated in FIG. 15. This indicated that these enzymes have higher overall activity at the highest temperature tested (65° C.).

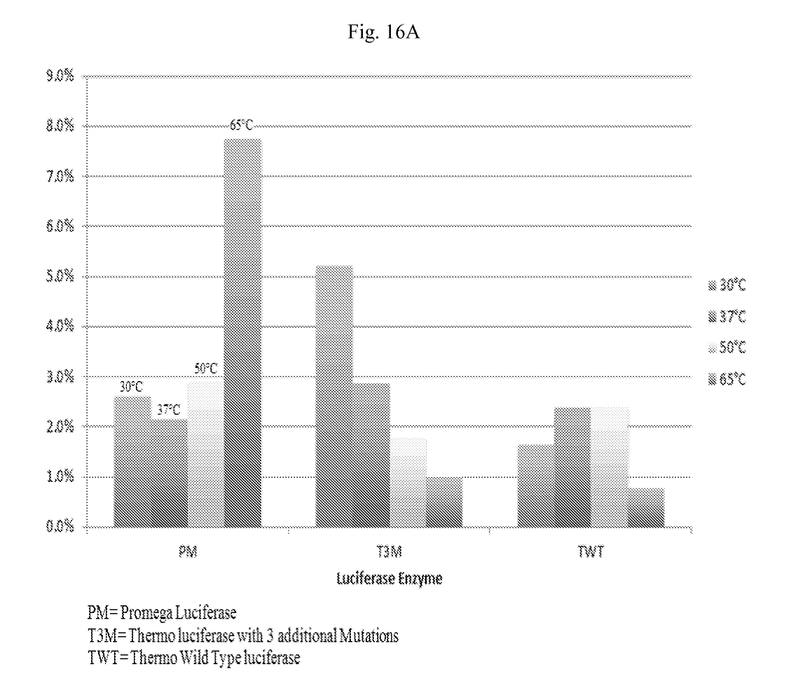

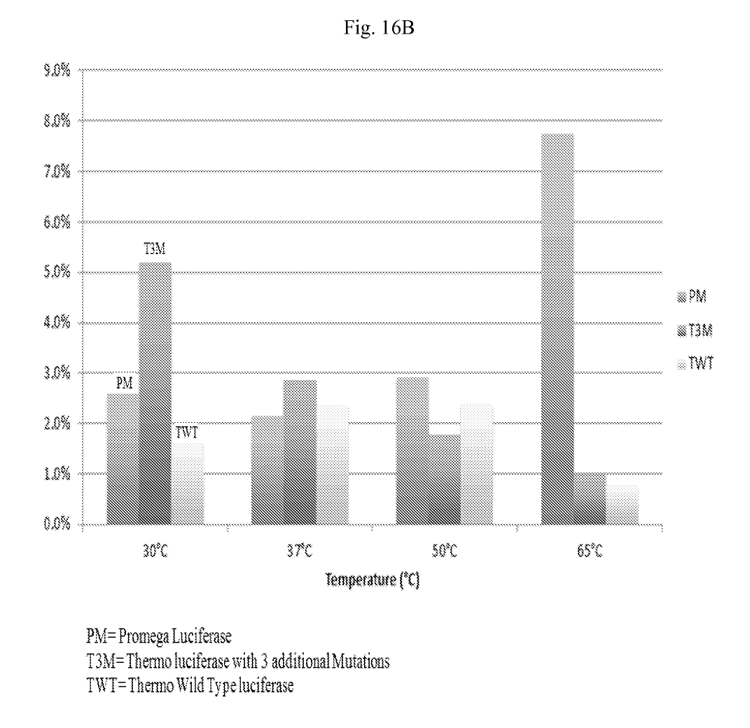

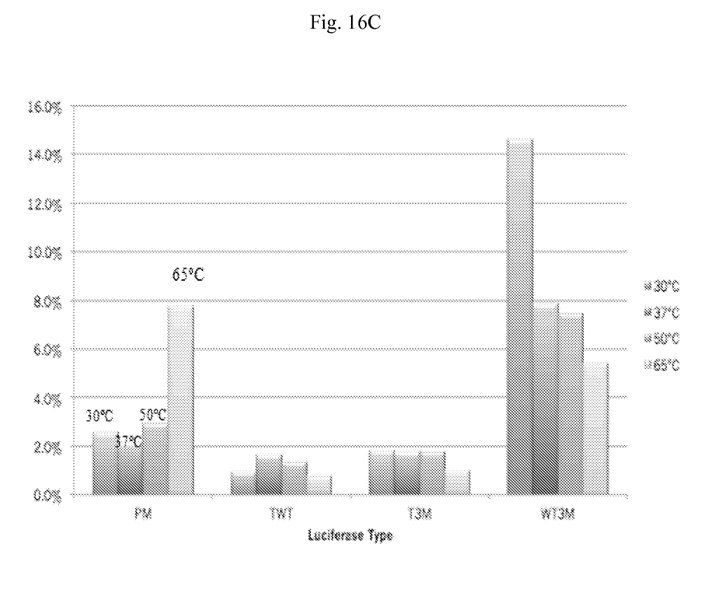

FIG. 16A-FIG. 16C show the dATP activity of exemplary modified luciferase as a percentage of its ATP activity at various temperatures. Promega Luciferase was used as a control.

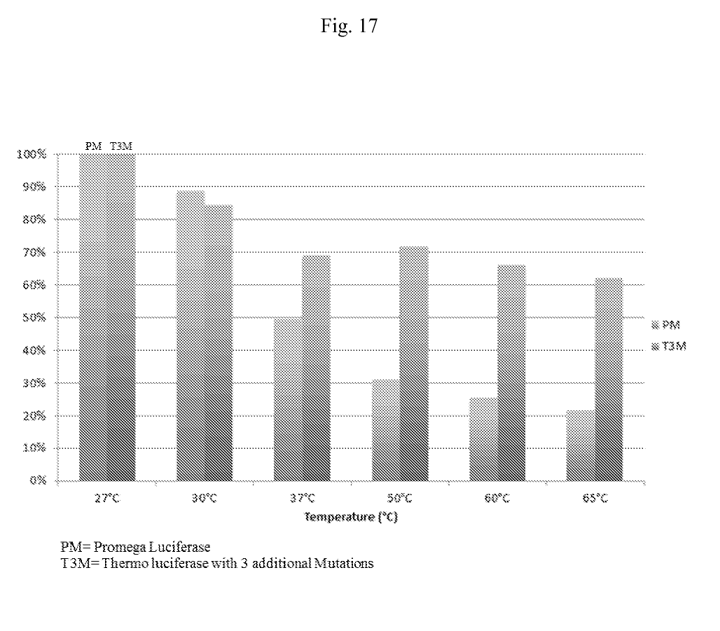

FIG. 17 illustrates comparison of luciferase activity as a percentage of its original activity at 27° C. between Promega Luciferase and an exemplary modified luciferase described herein. As temperature increased from 27° C.-65° C., the activity of Promega Luciferase decreased to about 20% while the activity of the exemplary modified luciferase decreased to about 60%.

### Example 7

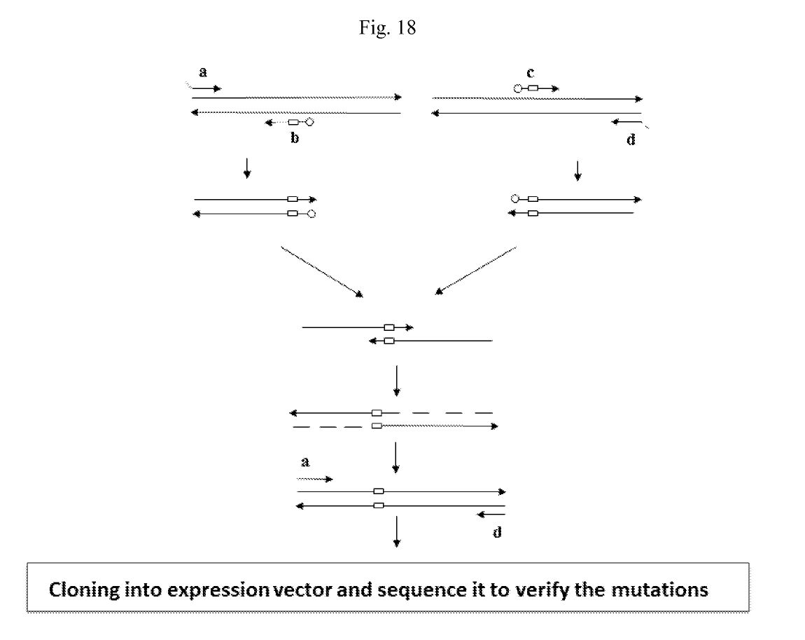

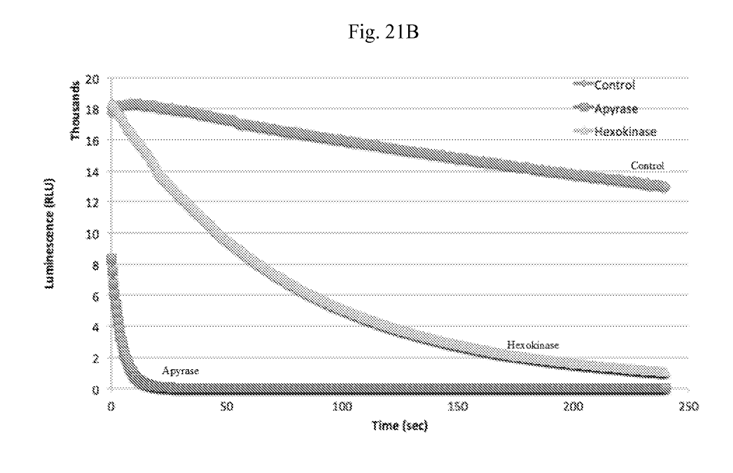

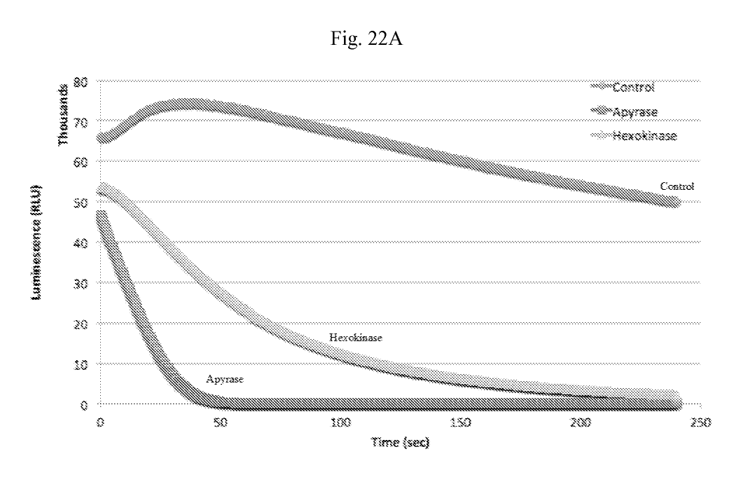

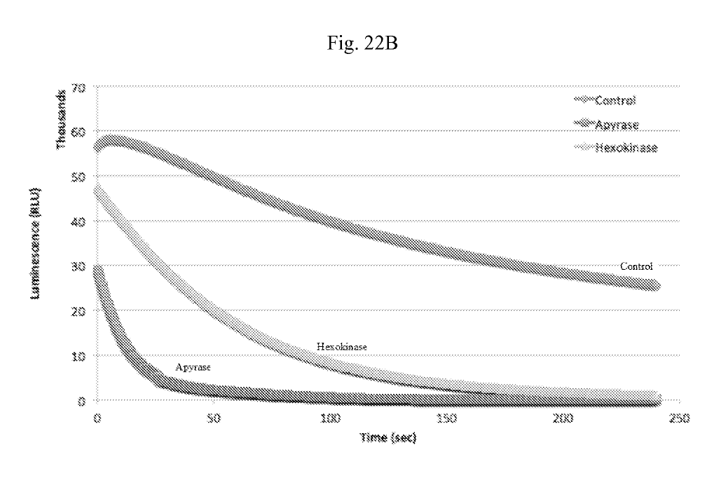

Wild Type Hexokinase Pichia pastoris was cloned and expressed in E. coli, Saccharomyces cerevisiae or Pichia pastoris. FIG. 18 illustrates an exemplary PCR mutagenesis of hexokinase. FIG. 19 illustrates exemplary hexokinase constructs described herein. FIG. 20A-FIG. 20C illustrate exemplary luciferase and hexokinase construct designs comprising Z-basic tag. FIG. 21A-FIG. 21B illustrate apyrase and hexokinase activity on dATP at 27° C. (A) and 50° C. (B). FIG. 22A-FIG. 22B show apyrase and hexokinase activity on dNTP at 27° C. (A) and 50° C. (B).

### Example 8

The following illustrates protein and nucleic acid sequences of Luciferase and Hexokinase described herein.

SEQ ID NOS: 1 and 2 provide an exemplary Firefly Luciferase Photinus pyralis nucleic acid and protein sequences, respectively.

SEQ ID NO: 1
atggaagatgcgaaaaacattaaaaaaggcccggcgccgttttatccgct
ggaagatggcaccgcgggcgaacagctgcataaagcgatgaaacgctatg
cgctggtgccgggcaccattgcgtttaccgatgcgcatattgaagtgaac
attacctatgcggaatattttgaaatgagcgtgcgcctggcggaagcgat
gaaacgctatggcctgaacaccaaccatcgcattgtggtgtgcagcgaaa
acagcctgcagttttttatgccggtgctgggcgcgctgtttattggcgtg
gcggtggcgccggcgaacgatatttataacgaacgcgaactgctgaacag
catgaacattagccagccgaccgtggtgtttgtgagcaaaaaaggcctgc
agaaaattctgaacgtgcagaaaaaactgccgattattcagaaaattatt
attatggatagcaaaaccgattatcagggattcagagcatgtataccttt
gtgaccagccatctgccgccgggattaacgaatatgattttgtgccggaa
agctttgatcgcgataaaaccattgcgctgattatgaacagcagcggcag
caccggcagcccgaaaggcgtggcgctgccg catcgcaccgcgtgcgtg
cgctttagccatgcgcgcgatccgatttttggcaaccagattattccgga
taccgcgattctgagcgtggtgccgtttcatcatggattggcatgtttac
caccctgggctatctgatttgcggattcgcgtggtgctgatgtatcgctt
tgaagaagaactgtttctgcgcagcctgcaggattataaaattcagagcg
cgctgctggtgccgaccctgtttagcttttttgcgaaaagcaccctgatt
gataaatatgatctgagcaacctgcatgaaattgcgagcggcggcgcgcc
gctgagcaaagaagtgggcgaagcggtggcgaaacgctttcatctgccgg
gcattcgccagggctatggcctgaccgaaaccaccagcgcgattctgatt
accccggaaggcgatgataaaccgggcgcggtgggcaaagtggtgccgtt
ttttgaagcgaaagtggtggatctggataccggcaaaaccctgggcgtga
accagcgcggcgaactgtgcgtgcgcggcccgatgattatgagcggctat
gtgaacgatccggaagcgaccaacgcgctgattgataaagatggctggct
gcatagcggcgatattgcgtattgggatgaagatgaacatttttttattg
tggatcgcctgaaaagcctgattaaatataaaggctgccaggtggcgccg
gcggaactggaaagcattctgctgcagcatccgaacatttttgatgcggg
cgtggcgggcctgccgggcgatgatgcgggcgaactgccggcggcggtgg
tggtgctggaacatggcaaaaccatgaccgaaaaagaaattgtggattat
gtggcgagccaggtgaccaccgcgaaaaaactgcgcggcggcgtggtgtt
tgtggatgaagtgccgaaaggcctgaccggcaaactggatgcgcgcaaaa
ttcgcgaaattctgattaaagcgaaaaaaggcggcaaaagcaaactg
SEQ ID NO: 2
MetEDAKNIKKGPAPFYPLEDGTAGEQLHKAMetKRYALVP
GTIAFTDAHIEVNITYAEYFEMetSVRLAEAMetKRYGLNTNHRI
VVCSENSLQFFMetPVLGALFIGVAVAPANDIYNERELLNSMetN
ISQPTVVFVSKKGLQKILNVQKKLPIIQKIIIMetDSKTDYQGFQS
MetYTFVTSHLPPGFNEYDFVPESFDRDKTIALIMetNSSGSTGSP
KGVALPHRTACVRFSHARDPIFGNQIIPDTAILSVVPFHHGFG
MetFTTLGYLICGFRVVLMetYRFEEELFLRSLQDYKIQSALLVPT
LFSFFAKSTLIDKYDLSNLHEIASGGAPLSKEVGEAVAKRFHLP
GIRQGYGLTETTSAILITPEGDDKPGAVGKVVPFFEAKVVDLD
TGKTLGVNQRGELCVRGPMetIMetSGYVNDPEATNALIDKDGW
LHSGDIAYWDEDEHFFIVDRLKSLIKYKGCQVAPAELESILLQ
HPNIFDAGVAGLPGDDAGELPAAVVVLEHGKTMetTEKEIVDY
VASQVTTAKKLRGGVVFVDEVPKGLTGKLDARKIREILIKAKK
GGKSKL

SEQ ID NOS: 3 and 4 provide an exemplary Pichia pastoris Hexokinase gene and protein sequences, respectively.

SEQ ID NO: 3
atggttcacttaggggcgaagaagcctcagcatagaaaaggatatctat
ttaatcagcttagtccagagttacgaaaagcttataaagaagtagaggc
acagttcgttgtatcaactcccaggttaaagcagatagtggatcaattt
gttgctgaattgaaggaaggcttgaaatcaagcagctctaacatcccaa
tgctacctacctgggtgatggatttccccacaggagaggaaactggaga
ctatctcgcaattgacctgggcgggaccaatataagggttatattggtc
agactgttgggaaataggaagtttgataccattcaatcaaaatatgttt
tgcctaaatggatcagaacctccacatcaaatgaactttggctttttat
tgctcaatgtgtgaagactttcattgatgaagagtttgattatagagaa
agtccggaagacccaatccccttagggttcacattttcttaccctgcat
tccaaagtaggattaattcgggtgtcttacaacgttggacgaaaggatt
tgatattccggacgttgaaggccatgacgttgtccctatgctgcaagac
gcattggaatcattgggattgtccgttgtggtagtggctttaattaatg
acactacaggcactttggtagatctacgtatacggatccagagactaaa
atgggactgatatttggaactggtgttaatggtgcatactatgatacga
taagttcggtctcaaagatatcaaatgctcttccaccagatattcaaga
ggatgcaagtatggccatcaactgcgaatacggtgcttttgacaataac
atctcagttctacctaggaccaaatacgatgatacaattgatttagaat
cgcccagaccagggcaacagtcgtatgaaaaaatgatatcaggctatta
tcttggagaattgttacgattggtgcttgtagatttgcaccatcaagga
cacattttcaagggacaaacaatcgggaaactaaatgaaccgttcatta
tggatacatcctttcctgcgagaattgaagaggatccgtttgagaatct
atgtgagactggagaactttttaacagcctaggaattgaaactacggtt
cccgaaagagaactgattagacgaatttgcgaactcataggaacaagag
cagccagactgtcagtatgtagcattgccgcaatttgcaaaaaacgagg
ctacaagaaagcccattgtgctgctgatggctctgtcttcacccgctat
ccttatttccctgacagagctgcaagagcgctccgagatatattccaat
ggggccattcaactccggacttagttactgtagttccagcagaagatgg
ctcgggagttggggcagctattattgctgcactgaccaaacaacggatg
gcaaatggtgaatccgtgggccttgacgaatatcaccctcaagatggaa
aggatagtaactaa
SEQ ID NO: 4
MetVHLGAKKPQHRKGYLFNQLSPELRKAYKEVEAQFVVS
TPRLKQIVDQFVAELKEGLKSSSSNIPMetLPTWVMetDFPTGEET
GDYLAIDLGGTNIRVILVRLLGNRKFDTIQSKYVLPKWIRTSTS
NELWLFIAQCVKTFIDEEFDYRESPEDPIPLGFTFSYPAFQSRIN
SGVLQRWTKGFDIPDVEGHDVVPMetLQDALESLGLSVVVVAL
INDTTGTLVASTYTDPETKMetGLIFGTGVNGAYYDTISSVSKIS
NALPPDIQEDASMetAINCEYGAFDNNISVLPRTKYDDTIDLESP
RPGQQSYEKMetISGYYLGELLRLVLVDLHHQGHIFKGQTIGKL
NEPFIMetDTSFPARIEEDPFENLCETGELFNSLGIETTVPERELI
RRICELIGTRAARLSVCSIAAICKKRGYKKAHCAADGSVFTRY
PYFPDRAARALRDIFQWGHSTPDLVTVVPAEDGSGVGAAIIAA
LTKQRMetANGESVGLDEYHPQDGKDSNStop

SEQ ID NOS: 5 and 6 provide an exemplary Saccharomyces cerevisiae Hexokinase gene and protein sequences, respectively.

SEQ ID NO: 5
atggttcatttaggtccaaaaaaaccacaagccagaaagggttccatggc
cgatgtgccaaaggaattgatgcaacaaattgagaattttgaaaaaattt
tcactgttccaactgaaactttacaagccgttaccaagcatttcatttcc
gaattggaaaagggtttgtccaagaagggtggtaacattccaatgattcc
aggttgggttatggatttcccaactggtaaggaatccggtgatttcttgg
ccattgatttgggtggtaccaacttgagagttgtcttagtcaagttgggc
ggtgaccgtacctttgacaccactcaatctaagtacagattaccagatgc
tatgagaactactcaaaatccagacgaattgtgggaatttattgccgact
attgaaagcttttattgatgagcaattcccacaaggtatctctgagccaa
ttccattgggtttcaccttttctttcccagcttctcaaaacaaaatcaat
gaaggtatcttgcaaagatggactaaaggttttgatattccaaacattga
aaaccacgatgttgttccaatgttgcaaaagcaaatcaccaagaggaata
tcccaattgaagttgttgattgataaacgacactaccggtactttggttg
cttcttactacactgacccagaaactaagatgggtgttatcttcggtact
ggtgtcaatggtgcttactacgatgtttgttccgatatcgaaaagctaca
aggaaaactatctgatgacattccaccatctgctccaatggccatcaact
gtgaatacggttccttcgataatgaacatgtcgttttgccaagaactaaa
tacgatatcaccattgatgaagaatctccaagaccaggccaacaaaccif
igaaaaaatgtcttctggttactacttaggtgaaattttgcgtttggcct
tgatggacatgtacaaacaaggtttcatcttcaagaaccaagacttgtct
aagttcgacaagcctttcgtcatggacacttcttacccagccagaatcga
ggaagatccattcgagaacctagaagataccgatgacttgttccaaaatg
agttcggtatcaacactactgttcaagaacgtaaattgatcagacgttta
tctgaattgattggtgctagagctgctagattgtccgtttgtggtattgc
tgctatctgtcaaaagagaggttacaagaccggtcacatcgctgcagacg
gttccgtttacaacagatacccaggfficaaagaaaaggctgccaatgct
ttgaaggacatttacggctggactcaaacctcactagacgactacccaat
caagattgttcctgctgaagatggttccggtgctggtgccgctgttattg
ctgctttggcccaaaaaagaattgctgaaggtaagtccgttggtatcatc
ggtgcttaa
SEQ ID NO: 6
MVHLGPKKPQARKGSMADVPKELMQQIENFEKIFTVPTETLQAVTKHFIS
ELEKGLSKKGGNIPMIPGWVMDFPTGKESGDFLAIDLGGTNLRVVLVKLG
GDRTFDTTQSKYRLPDAMRTTQNPDELWEFIADSLKAFIDEQFPQGISEP
IPLGFTFSFPASQNKINEGILQRWTKGFDIPNIENHDVVPMLQKQITKRN
IPIEVVALINDTTGTLVASYYTDPETKMGVIFGTGVNGAYYDVCSDIEKL
QGKLSDDIPPSAPMAINCEYGSFDNEHVVLPRTKYDITIDEESPRPGQQT
FEKMSSGYYLGEILRLALMDMYKQGFIFKNQDLSKFDKPFVMDTSYPARI
EEDPFENLEDTDDLFQNEFGINTTVQERKLIRRLSELIGARAARLSVCGI
AAICQKRGYKTGHIAADGSVYNRYPGFKEKAANALKDIYGWTQTSLDDYP
IKIVPAEDGSGAGAAVIAALAQKRIAEGKSVGIIGA*

The examples and embodiments described herein are for illustrative purposes only and various modifications or changes suggested to persons skilled in the art are to be included within the spirit and purview of this application and scope of the appended claims.

What is claimed is:

1. A method for measuring pyrophosphate released during a polynucleotide replication process comprising:
a. performing polynucleotide replication in a reaction mixture, wherein the replication results in a release of at least one pyrophosphate;
b. converting the pyrophosphate into ATP;
c. adding at least one saccharide to the reaction mixture; and
d. detecting the ATP using a thermostable luciferase that has an impaired recognition of dATP.

2. The method of claim 1, wherein the adding at least one saccharide follows the polynucleotide replication.

3. The method of claim 2, wherein the saccharide is phosphorylated.

4. The method of claim 3, wherein phosphorylation of the saccharide quenches an excess of nucleotides.

5. The method of claim 4, wherein the phosphorylation of the saccharide comprises using a saccharide phosphorylating enzyme.

6. The method of claim 1, wherein the thermostable luciferase is a thermostable firefly luciferase.

7. The method of claim 1 or 6, wherein the thermostable luciferase comprises a modification at one or more positions corresponding to amino acid residues T214, I232, F295, E354, I423, D436, L530, and L550 of SEQ ID NO: 2.

8. The method of claim 7, wherein the modifications include T214A, I232A, F295L, E354K, I423L, D436G, L530R, and L550V.

9. The method of claim 8, wherein the modifications include T214A, I232A, F295L, I423L, and L550V.

10. The method of claim 1, wherein the impaired recognition of the thermostable luciferase is a decrease in affinity toward dATP.

11. The method of any one of the claim 1 or 6-10, wherein the thermostable luciferase does not recognize dATP and recognizes ATP.

12. The method of any one of the claim 1 or 6-11, wherein the thermostable luciferase further comprises a binding protein selected from albumin binding protein or Z domain.

13. The method of claim 1, wherein the converting is conducted in the presence of ATP sulfurylase.

14. The method of claim 13, wherein the ATP sulfurylase is thermostable.

15. The method of claim 1, wherein the polynucleotide replication further comprises the steps of
a. hybridizing a complementary polynucleotide that is complementary to at least a portion of at least one target polynucleotide to the at least one target polynucleotide;
b. hybridizing one species of nucleoside polyphosphate to the at least one target polynucleotide, wherein the nucleoside is selected from the group consisting of adenine, thymine, guanine, cytosine, and uracil; and
c. linking the one species of nucleoside polyphosphate with the complementary polynucleotide to elongate the complementary polynucleotide.

16. The method of claim 1, wherein the polynucleotide replication is performed at a temperature greater than 50 degrees Celsius.

17. The method of claim 1, wherein the polynucleotide replication is performed at a temperature that is at least 50 degrees Celsius, at least 55 degrees Celsius, at least 60 degrees Celsius, at least 65 degrees Celsius, or at least 70 degrees Celsius.

18. The method of claim 1 or 15, wherein the polynucleotide replication is conducted in the presence of a polymerase.

19. The method of claim 18, wherein the polymerase is Taq polymerase.

20. The method of claim 19, wherein the Taq polymerase is native Taq polymerase, recombinant Taq polymerase, or modified Taq polymerase.

21. The method of claim 1, wherein the method excludes the use of a single strand binding protein (SSB).

22. The method of claim 5, wherein the saccharide phosphorylating enzyme is hexokinase.

23. The method of claim 22, wherein the hexokinase is a modified hexokinase further comprising a binding protein selected from albumin binding protein or Z domain.

24. The method of claim 22 or 23, wherein the hexokinase is expressed in Saccharomyces cerevisiae, Pichia pastoris, or E. coli.

25. The method of any one of the claims 22-24, wherein the hexokinase is a thermostable hexokinase.

26. The method of claim 12 or 23, wherein the albumin binding protein comprises ABP (121aa), BB (214aa), ABD (46aa), ADB1 binding site, ADB2 binding site, or ADB3 binding site.

27. The method of claim 26, wherein the ABD to albumin affinity is 1.5 nanomolar or less.

28. The method of claim 27, wherein the albumin is serum albumin.

29. The method of claim 27, wherein the albumin is human serum albumin.

30. The method of any one of the claim 1, 6-12, or 22-25, wherein the hexokinase, the luciferase or the ATP sulfurylase are chemically modified.

31. The method of claim 30, wherein the chemically modified hexokinase, luciferase, or ATP sulfurylase comprises chemical neutralization or chemical acidification of the basic side chains of the hexokinase, luciferase or sulfurylase.

32. The method of claim 30, wherein the chemically modified hexokinase, luciferase, or ATP sulfurylase comprises acetylation or citraconylation.

33. A thermostable luciferase that facilitates a bioluminescent reaction and has an impaired recognition of dATP as a substrate.

34. The thermostable luciferase of claim 33, wherein the thermostable luciferase comprises a modification at one or more positions corresponding to amino acid residues T214, I232, F295, E354, I423, D436, L530, and L550 of SEQ ID NO: 2.

35. The thermostable luciferase of claim 34, wherein the modifications include T214A, I232A, F295L, E354K, I423L, D436G, L530R, and L550V.

36. The thermostable luciferase of claim 35, wherein the modifications include T214A, I232A, F295L, I423L, and L550V.

37. The thermostable luciferase of claim 33, wherein the thermostable luciferase is further chemically modified.

38. The thermostable luciferase of claim 37, wherein the chemically modified luciferase comprises chemical neutralization or chemical acidification of the basic side chains of luciferase.

39. The thermostable luciferase of claim 37, wherein the chemically modified luciferase comprises acetylation or citraconylation.

40. The thermostable luciferase of any one of the claims 33-39, wherein the thermostable luciferase is used in a method of claims 1-32.

41. A bioluminescent enzyme construct comprising a bioluminescent enzyme and a binding moiety selected from albumin binding protein or Z domain.

42. The enzyme construct of claim 41, wherein the bioluminescent enzyme is luciferase.

43. The enzyme construct of claim 42, wherein the luciferase comprises a modification at one or more positions corresponding to amino acid residues T214, I232, F295, E354, I423, D436, L530, and L550 of SEQ ID NO: 2.

44. The enzyme construct of claim 43, wherein the modifications include T214A, I232A, F295L, E354K, I423L, D436G, L530R, and L550V.

45. The enzyme construct of claim 44, wherein the modifications include T214A, I232A, F295L, I423L, and L550V.

46. The enzyme construct of claim 41, wherein the albumin binding protein comprises BB, ABD, ABP, ABD1, ABD2, or ABD3.

47. The enzyme construct of claim 41, further comprising a His(6) moiety.

48. The enzyme construct of claim 47, wherein the binding moiety sequence is connected at the 5′ of the bioluminescent enzyme sequence and further connected to the 3′ of the His(6) sequence, or is connected at the 5′ of the HIS(6) sequence and further connected to the 3′ of the bioluminescent enzyme sequence.

49. The enzyme construct of claim 47, wherein the HIS(6) sequence is connected at the 5′ of the bioluminescent enzyme sequence and further connected to the 3′ of the binding moiety sequence, or is connected at the 5′ of the binding moiety sequence and further connected to the 3′ of the bioluminescent enzyme sequence.

50. The enzyme construct of claim 47, wherein the bioluminescent enzyme sequence is connected at the 5′ of the binding moiety sequence and further connected to the 3′ of the His(6) sequence, or is connected at the 5′ of the HIS(6) sequence and further connected to the 3′ of the binding moiety sequence.

51. A saccharide phosphorylating enzyme construct comprising a binding moiety selected from albumin binding protein or Z domain, wherein the saccharide phosphorylating enzyme phosphorylates at least one saccharide to produce at least one phosphorylated saccharide.

52. The enzyme construct of claim 51, wherein the saccharide phosphorylating enzyme is hexokinase.

53. The enzyme construct of claim 51, wherein the albumin binding protein comprises ABP (121aa), BB (214aa), ABD (46aa), ADB1 binding site, ADB2 binding site, or ADB3 binding site.

54. The enzyme construct of claim 53, wherein the ABD to albumin affinity is 1.5 nanomolar or less.

55. The enzyme construct of claim 54, wherein the albumin is human serum albumin or bovine serum albumin.

56. The enzyme construct of claim 52, wherein the enzyme construct further comprises a HIS(6) moiety.

57. The enzyme construct of claim 56, wherein the enzyme construct comprises a construct depicted in FIG. 5, 19 or 20C.

58. The enzyme construct of claim 52, wherein the enzyme construct is expressed in Saccharomyces cerevisiae, Pichia pastoris or E. coli.

59. A saccharide phosphorylating enzyme comprising a binding moiety selected from albumin binding protein or Z domain, wherein the enzyme has similar binding affinities for at least two nucleotides selected from the list consisting of dTTP, dCTP, dGTP, dUTP and dATP.

60. The enzyme of claim 59, wherein the enzyme phosphorylates at least one saccharide to produce at least one phosphorylated saccharide.

61. The enzyme of claim 60, wherein similar binding affinities range from at least 1 micromolar to at most 250 micromolar.

62. The enzyme of claim 59, wherein the enzyme reacts the nucleotides with at least one saccharide with a similar efficiency.

63. The enzyme of claim 59, wherein the enzyme reacts the nucleotides with at least one saccharide in a similar rate.

64. The enzyme of claim 62 or 63, wherein the saccharide is hexose.

65. The enzyme of claim 64, wherein the hexose is selected from the group consisting of glucose, allose, altrose, mannose, gulose, idose, galactose and talose.

66. The enzyme of any one of the claims 59-65, wherein the enzyme is hexokinase.

67. The enzyme of claim 66, wherein hexokinase comprise a construct of claims 51-58.

68. The enzyme of claim 66 or 67, wherein hexokinase is further chemically modified.

69. The enzyme of claim 68, wherein the chemically modified hexokinase comprises chemical neutralization or chemical acidification of the basic side chains of luciferase.

70. The enzyme of claim 68, wherein the chemically modified hexokinase comprises acetylation or citraconylation.

71. The enzyme of any one of the claims 68-70, wherein the chemically modified hexokinase is a thermostable hexokinase.

72. The enzyme of any one of the claims 59-71, wherein the saccharide phosphorylating enzyme is used in a method of claims 1-32.
